(19) United States
(12) Patent Application Publication
(10) Pub. No.: US 2021/0221864 A1
(43) Pub. Date: Jul. 22, 2021

(54) MASKED CYTOKINE CONJUGATES

(71) Applicant: CITY OF HOPE, Duarte, CA (US)

(72) Inventors: John C. WILLIAMS, Duarte, CA (US); Miso PARK, Duarte, CA (US)

(21) Appl. No.: 17/270,795

(22) PCT Filed: Aug. 23, 2019
(86) PCT No.: PCT/US19/48004

§ 371 Date: Feb. 23, 2021

Related U.S. Application Data
(60) Provisional application No. 62/722,806, filed on Aug. 24, 2018.
(60) Provisional application No. 62/731,772, filed on Sep. 14, 2018.

(51) Int. Cl. C07K 14/55; C07K 14/54; C07K 14/715; C07K 16/00; A61P 35/00

(52) U.S. Cl. CPC C07K 14/55; C07K 14/5443; A61K 38/00; C07K 16/00; A61P 35/00; C07K 14/7155

(57) ABSTRACT

Provided herein are, inter alia, recombinant cytokine receptor binding proteins including a cytokine domain bound to an occlusion domain and a receptor domain through a first and second linker, respectively. The occlusion domain hinders the cytokine domain from binding to its cognate receptor. The chemical linkers (e.g., first and/or second chemical linker) included in the recombinant proteins provided herein may be cleavable and thereby conveying disease site specificity to the compositions provided herein. In the presence of a tumor-specific protease the first and/or second chemical linker is cleaved and the cytokine domain is released from the occlusion domain and the receptor domain and is capable of binding its cognate receptor. The recombinant proteins provided herein may therefore have multi-specific binding capabilities useful for therapeutic and diagnostic purposes.

### CROSS-REFERENCE TO RELATED APPLICATIONS

This application claims priority to U.S. Provisional Application No. 62/722,806 filed Aug. 24, 2018, and U.S. Provisional Application No. 62/731,772 filed Sep. 14, 2018. The contents of the above-listed applications are incorporated herein by this reference in their entirety for all purposes.

### INCORPORATION BY REFERENCE OF SEQUENCE LISTING

The present application is being filed along with a Sequence Listing in electronic format. The Sequence Listing is provided as a file entitled 737762001940SeqList, created 22 Aug. 2019, which is 278 KB in size. The information in the electronic format of the Sequence Listing is incorporated by reference in its entirety.

### BACKGROUND

There is a need in the art for compositions and methods of delivering anti-cancer therapeutics to the site of the cancer without causing off-tissue toxicities and resulting in undesirable adverse side effects. Provided herein are, inter alia, cytokine receptor binding proteins and methods of using the same that address these and other needs in the art.

### BRIEF SUMMARY OF THE INVENTION

In an aspect is provided a recombinant cytokine receptor binding protein including: (i) a cytokine domain including a first receptor binding site and a second receptor binding site; (ii) a first receptor domain that specifically binds to the first receptor binding site; and (iii) an occlusion domain positioned to sterically hinder binding of the second receptor binding site to a cognate receptor of the second receptor binding site; wherein the C-terminus of the cytokine domain is bound to the N-terminus of the occlusion domain through a first chemical linker; and the C-terminus of the occlusion domain is bound to the N-terminus of the first receptor domain through a second chemical linker.

In another aspect is provided a recombinant cytokine receptor binding protein including: (i) a cytokine domain including a first receptor binding site and a second receptor binding site; (ii) a first receptor domain that specifically binds to the first receptor binding site; and (iii) an occlusion domain positioned to sterically hinder binding of the second receptor binding site to a cognate receptor of the second receptor binding site; wherein the C-terminus of the occlusion domain is bound to the N-terminus of the cytokine domain through a first chemical linker; and the C-terminus of the first receptor dominator is bound to the N-terminus of the occlusion domain through a second chemical linker.

In another aspect is provided a recombinant cytokine receptor binding protein including: (i) an IL-2 domain comprising an IL-2 receptor α binding site, an IL-2 receptor β binding site and an IL-2 receptor γ binding site; (ii) an IL-2 receptor α domain that specifically binds to the IL-2 receptor α binding site; and (iii) an Fc domain positioned to sterically hinder binding of the IL-2 receptor β binding site to an IL-2 receptor β domain; wherein the C-terminus of the IL-2 domain is bound to the N-terminus of the Fc domain through a cleavable linker; and the C-terminus of the Fc domain is bound to the N-terminus of the IL2 receptor α domain through a chemical linker.

In another aspect, a pharmaceutical composition is provided. The pharmaceutical composition includes a recombinant cytokine receptor binding protein provided herein including embodiments thereof and a pharmaceutically acceptable excipient.

In another aspect, an isolated nucleic acid is provided. The nucleic acid encodes a recombinant cytokine receptor binding protein provided herein including embodiments thereof.

In another aspect, an expression vector including the nucleic acid provided herein including embodiments thereof is provided.

In another aspect, a T lymphocyte including the expression vector provided herein including embodiments thereof is provided.

In another aspect is provided a method of treating cancer including administering to a subject in need thereof a therapeutically effective amount of a recombinant cytokine receptor binding protein as described herein, including embodiments thereof.

### BRIEF DESCRIPTION OF THE DRAWINGS

FIG. 1. The figure shows a proposed mechanism of action, as well as design to maintain FcR binding by CH2 domain.

FIG. 2. The figure illustrates a phaging opportunity to improve alignment, using a cavity to build from. This could be used to make a short library off of the N-terminus of IL2a.

FIGS. 3A-3D. The figures show a new masked cytokine design and production. FIG. 3A shows a schematic of the construct: human IL2 (“hIL2”), a first chemical linker (SEQ ID NO: 23) that includes protease cleavage site (MMP2/9) (VPLSLY) (SEQ ID NO: 29), the CH2 domain of human IgG1—a second chemical linker that is a non-cleavable linker (GASASGGG) (SEQ ID NO: 10)—IL2Ralpha domain—and a short spacer linked to the His tag (ASGGGGHHHHHH) (SEQ ID NO: 57). As indicated in FIG. 3A, an exemplary sequence for the 329141 construct includes SEQ ID NO: 6. FIG. 3B shows a depiction of the design. FIG. 3C shows SDS-PAGE gel—MW markers is the middle lane (75 kD and 25 kD). The left lane is non-reduced and the right is reduced. The expected mass is 49625 D. FIG. 3D shows size exclusion chromatography of the IL2-CH2-IL2Ra construct.

FIGS. 4A and 4B. FIG. 4A shows a cell proliferation assay of CTLL2 mouse cytotoxic T cell line with new masked cytokine (329141). An in vitro assay was used to compare the activity of the masked (closed triangles) and the activated IL2-CH2-CH2Ra construct (open triangles). As a control, commercial IL2 (from R&D Systems) was also used (open circle). As shown in FIG. 4B, a 3 fold difference is observed in the EC50 between the masked (329141 null; closed triangle) and the activated (329141 mmp1; open triangle) 329141 construct. More on the assay can be found at website: assets.thermofisher.com/TFS-Assets/LS G/manuals/irfl_CTLL2-Validation-Packet.pdf.

FIGS. 5A and 5B depicts an SDS-PAGE gel of the exemplary 329141 construct after exposure to each of MMPs 1, 2, 7, 10, and 14 (FIG. 5A) or each of MMPs 1, 2, 7, 9, 10, and 14 (FIG. 5B), or no exposure to a protease (null). Under reduced conditions, the construct is expected to be approximately 49 kDa in size, and after cleavage, it is expected to include cleavage products of approximately 33 kDa and 16 kDa in size.

FIG. 6 depicts an SDS-PAGE analysis of the exemplary constructs termed 329141A (“A”), 329141B (“B”), 329141C (“C”), and 329141D (“D”) under non-reduced (left) and reduced (right) conditions using samples where the size-exclusion chromatography (SEC) fraction was removed (“F1”) or used (“F2”).

FIG. 7 depicts an SDS-PAGE analysis of IL2 from the exemplary constructs 320141 and 329141D with (+) or without (−) previously being exposed to the MMP10 protease, as well as recombinant human IL2 (rhIL2) as a control.

FIGS. 8A-8C depict the results from an SPR analysis showing binding between the 329141 construct, or rhIL2 as a control, and rhCD25-Fc at concentrations ranging from 1 nM to 16 nM of rhCD25-Fc as analyte. FIG. 8C provides data on the association rate (ka), dissociation rate (kd), and equilibrium dissociation constant (KD), as well as Chi² and U-values for the interaction between rhCD25-Fc and either the 329141 construct or rhIL2.

FIGS. 9A-9C depict the results from an SPR analysis showing binding between the 329141 construct, or rhIL2 as a control, and rhFc-CD122 at concentrations ranging from 31.25 nM to 500 nM of rhFc-CD122 as analyte. FIG. 9C provides data on the association rate (ka), dissociation rate (kd), and equilibrium dissociation constant (KD), as well as Chi² and U-values for the interaction between rhFc-CD122 and either the 329141 construct or rhIL2.

FIGS. 10A-10D depict the results from an analysis performed using size exclusion chromatography (SEC) on the constructs 329141A, 329141B, 329141C, and 329141D.

FIG. 11 depicts a 3D modeling of the exemplary 329141 construct, with arrows pointing to a CH2 domain, IL-2 domain, and CD25 (IL-2Rα) domain.

FIGS. 12A and 12B depict the results from a cell proliferation assay of the CTLL2 mouse cytotoxic T cell line, where the cell line was cultured in the presence of each of the constructs (329141A, 329141B, 329141C, and 329141D), that were either previously exposed to the MMP7 protease (“mmp7”) or were not previously exposed to the MMP7 protease (“null”). The cell line was also cultured in the presence of IL2 as a control. FIG. 12B provides data on EC50 values in pM that were calculated.

FIGS. 13A-13C depict the results from a Stat5 activation assay performed using human peripheral blood lymphocytes, including CD8+ T cells (FIG. 13A), CD4+ T cells (FIG. 13B), and NK cells (FIG. 13C), exposed to the 329141 or 329141D construct, that was unactivated or activated by MMP10 (“+MMP). rhlL-2 that was stored at 4 degrees or treated with MMP overnight (“37 degrees C.”) was also included as a control. A “no treatment” control was also included. The legend shown in FIG. 13A also applies to FIGS. 13B and 13C. The percentage of phosphorylated Stat5 (pStat5) cells relative to total cells is shown.

FIGS. 14A and 14B depict SDS-PAGE analyses of exemplary constructs 1022, 1023, 1027, 1028, and 329141D following incubation with MMP1, MMP2, MMP7, MMP9, or MMP10, or with no protease (0) as a control.

FIGS. 15A and 15B depict the results from a cell proliferation assay of the CTLL2 mouse cytotoxic T cell line, where the cell line was cultured in the presence of each of the constructs (1022, 1023, and 1027), that were either previously exposed to the MMP7 protease (“mmp7”) or were not previously exposed to the MMP7 protease (“null”). The cell line was also cultured in the presence of IL2 as a positive control, and in the presence of IL2Rα-Fc as a negative control. FIG. 15B provides data on EC50 values in pM that were calculated.

FIGS. 16A and 16B depict the results from a cell proliferation assay of the CTLL2 mouse cytotoxic T cell line, where the cell line was cultured in the presence of the 1028 construct or the 329141D construct, in conditions where the construct was either previously exposed to the MMP7 protease (“mmp7”) or was not previously exposed to the MMP7 protease (“null”). The cell line was also cultured in the presence of IL2 as a positive control, and in the presence of IL2Rα-Fc as a negative control. FIG. 16B provides data on EC50 values in pM that were calculated.

FIGS. 17A and 17B depict SDS-PAGE analyses of exemplary constructs 1024, 1026, 1029, and 1030 following incubation with MMP1, MMP2, MMP7, MMP9, or MMP10, or with no protease (θ) as a control.

FIGS. 18A and 18B depict the results from a cell proliferation assay of the CTLL2 mouse cytotoxic T cell line, where the cell line was cultured in the presence of each of the tested constructs (1024, 1026, 1029, and 1030), in conditions where the construct was either previously exposed to the MMP7 or MMP1 protease or was not previously exposed to the MMP7 or MMP1 protease (“null”). The cell line was also cultured in the presence of IL2 as a control. FIG. 18B provides data on EC50 values in pM that were calculated.

FIGS. 19A-19C depict the results from an SPR analysis showing binding between select constructs (1022, 1023, 1024, 1029, and 1030), and a humanized IgG antibody (trastuzumab) at concentrations ranging from 3 nM to 300 nM of the construct as analyte. FIG. 19D provides data on the association rate (ka), dissociation rate (kd), and equilibrium dissociation constant (KD), as well as Chi² and U-values for the interaction between each construct and the humanized IgG antibody.

FIGS. 20A and 20B depict the results from an SPR analysis showing binding between select constructs (1022, 1023, 1024, 1029, and 1030), and a mouse IgG antibody at concentrations ranging from 3 nM to 300 nM of the construct as analyte.

FIG. 21 the results from an analysis performed using size exclusion chromatography (SEC) on the 1030 construct, a humanized IgG antibody (trastuzumab), and a combination of the 1030 construct and the humanized IgG antibody (trastuzumab) at a 1:1 molar ratio.

FIGS. 22A and 22B depict SDS-PAGE analyses of exemplary constructs 1023, 1024, and 1030, along with a humanized IgG antibody (trastuzumab), following incubation with MMP1 or MMP7, or with no protease (0) as a control. FIG. 22A depicts an SDS-PAGE analysis under non-reduced conditions, and FIG. 22B depicts an SDS-PAGE analysis under reduced conditions.

FIGS. 23A and 23B depict the results from a cell proliferation assay of the CTLL2 mouse cytotoxic T cell line, where the cell line was cultured in the presence of each of the tested constructs (1023, 1024, and 1030) along with a humanized IgG antibody (trastuzumab), in conditions where the construct was either previously exposed to the MMP7 or MMP1 protease or was not previously exposed to the MMP7 or MMP1 protease (“null”). The cell line was also cultured in the presence of IL2 as a control. FIG. 23B provides data on EC50 values in pM that were calculated.

FIG. 24 depicts an SDS-PAGE analysis of exemplary constructs 1031, 1032, and 1033 following incubation with MMP1, MMP2, MMP7, MMP9, or MMP10, or with no protease (0) as a control.

FIGS. 25A and 25B depict the results from a cell proliferation assay of the CTLL2 mouse cytotoxic T cell line, where the cell line was cultured in the presence of the exemplary 1032 construct or the 1033 construct, in conditions where the construct was either previously exposed to the MMP7 protease or was not previously exposed to the MMP7 protease (“null”). The cell line was also cultured in the presence of IL2 as a control. FIG. 25B provides data on EC50 values in pM that were calculated.

FIGS. 26A-26C depict the results from an SPR analysis showing binding between select constructs (1031, 1032, 1033, and 329141D), and a humanized IgG antibody (trastuzumab) at concentrations ranging from 3 nM to 300 nM of the 1031, 1032, and 1033 constructs as analyte, or at concentrations ranging from 30 nM to 300 nM for the 329141D construct. FIG. 26C provides data on the association rate (ka), dissociation rate (kd), and equilibrium dissociation constant (KD), as well as Chi² and U-values for the interaction between each construct and the humanized IgG antibody.

### DETAILED DESCRIPTION

### Cytokine Receptor Binding Proteins

Provided herein are, inter alia, recombinant cytokine receptor binding proteins including a cytokine domain bound to an occlusion domain and a receptor domain (e.g., first receptor domain) through a first and second linker, respectively. The occlusion domain hinders the cytokine domain from binding to its cognate receptor. The chemical linkers (e.g., first and/or second chemical linker) included in the recombinant proteins provided herein may be cleavable and thereby conveying disease site specificity to the compositions provided herein. For example, the first and/or second chemical linker may be a cleavable linker including a cleavage site recognized by a tumor-specific protease. In the absence of a tumor-specific protease the first and/or second chemical linker is not cleaved and the recombinant protein is in a sterically occluded conformation, wherein the cytokine domain does not bind its cognate receptor. In the presence of a tumor-specific protease the first and/or second chemical linker is cleaved and the cytokine domain is released from the occlusion domain and the receptor domain (e.g., first receptor domain) and is capable of binding its cognate receptor. Therefore, the recombinant proteins provided herein are highly effective in targeting tumor sites without causing off-tissue toxicities resulting in undesirable adverse side effects. Additional functionality (e.g., tumor-specific activation) can be included in the protein compositions through steric hindrance or masking of the cytokine domain and the ability of the occlusion domain to bind to cellular ligands. The recombinant proteins provided herein may therefore have multi-specific binding capabilities useful for therapeutic and diagnostic purposes. The protein compositions provided herein can be produced at very high yields and are therefore easy to manufacture.

The recombinant cytokine receptor binding proteins provided herein are single chain polypeptides that may include the cytokine domain, occlusion domain and receptor domain (e.g., first receptor domain) in varying order. The recombinant cytokine receptor binding protein may include from the N-terminus to the C-terminus: a cytokine domain, a first chemical linker, an occlusion domain, a second chemical linker and a first receptor domain. In other aspects, the recombinant cytokine receptor binding protein may include from the N-terminus to the C-terminus: a first receptor domain, a second chemical linker, an occlusion domain, a first chemical linker and a cytokine domain.

Thus, in an aspect is provided a recombinant cytokine receptor binding protein including: (i) a cytokine domain including a first receptor binding site and a second receptor binding site; (ii) a first receptor domain that specifically binds to the first receptor binding site; and (iii) an occlusion domain positioned to sterically hinder binding of the second receptor binding site to a cognate receptor of the second receptor binding site. The C-terminus of the cytokine domain is bound to the N-terminus of the occlusion domain through a first chemical linker; and the C-terminus of the occlusion domain is bound to the N-terminus of the first receptor domain through a second chemical linker.

In another aspect is provided a recombinant cytokine receptor binding protein including: (i) a cytokine domain including a first receptor binding site and a second receptor binding site; (ii) a first receptor domain that specifically binds to the first receptor binding site; and (iii) an occlusion domain positioned to sterically hinder binding of the second receptor binding site to a cognate receptor of the second receptor binding site. The C-terminus of the occlusion domain is bound to the N-terminus of the cytokine domain through a first chemical linker; and the C-terminus of the first receptor domain is bound to the N-terminus of the occlusion domain through a second chemical linker.

The cytokine domain including a first receptor binding site and a second receptor binding site may be an IL-2 domain or an IL-15 domain. Thus, in embodiments, the cytokine domain is an IL-2 domain. In some embodiments, the IL-2 domain comprises the amino acid sequence of SEQ ID NO: 1. In some embodiments, the IL-2 domain comprises an amino acid sequence that has at least 90%, 91%, 92%, 93%, 94%, 95%, 96%, 97%, 98%, or 99% amino acid sequence identity to the amino acid sequence of SEQ ID NO: 1 across the whole sequence or a portion of the sequence (e.g. a 50, 75, 100, or 125 continuous amino acid portion). In embodiments, the cytokine domain is an IL-15 domain. In some embodiments, the IL-15 domain comprises the amino acid sequence of SEQ ID NO: 92. In some embodiments, the IL-15 domain comprises an amino acid sequence that has at least 90%, 91%, 92%, 93%, 94%, 95%, 96%, 97%, 98%, or 99% amino acid sequence identity to the amino acid sequence of SEQ ID NO: 92 across the whole sequence or a portion of the sequence (e.g. a 50, 75, 100, or 125 continuous amino acid portion). Any cytokine domain including at least two receptor binding sites is contemplated for the invention provided herein. An IL-2 domain or IL-15 domain as provided herein includes at least 40 amino acid residues corresponding to 40 consecutive amino acid residues of IL-2 or IL-15, respectively. In embodiments, the cytokine domain is between 100 and 500 amino acid residues in length. In embodiments, the cytokine domain is between 110 and 500 amino acid residues in length. In embodiments, the cytokine domain is between 120 and 500 amino acid residues in length. In embodiments, the cytokine domain is between 130 and 500 amino acid residues in length. In embodiments, the cytokine domain is between 140 and 500 amino acid residues in length. In embodiments, the cytokine domain is between 150 and 500 amino acid residues in length. In embodiments, the cytokine domain is between 160 and 500 amino acid residues in length. In embodiments, the cytokine domain is between 170 and 500 amino acid residues in length. In embodiments, the cytokine domain is between 180 and 500 amino acid residues in length. In embodiments, the cytokine domain is between 190 and 500 amino acid residues in length. In embodiments, the cytokine domain is between 200 and 500 amino acid residues in length. In embodiments, the cytokine domain is between 250 and 500 amino acid residues in length. In embodiments, the cytokine domain is between 300 and 500 amino acid residues in length. In embodiments, the cytokine domain is between 350 and 500 amino acid residues in length. In embodiments, the cytokine domain is between 400 and 500 amino acid residues in length. In embodiments, the cytokine domain is between 450 and 500 amino acid residues in length. In embodiments, the cytokine domain is 100, 110, 120, 130, 140, 150, 160, 170, 180, 190, 200, 250, 300, 350, 400, 450, or 500 amino acid residues in length. In further embodiments, the 100, 110, 120, 130, 140, 150, 160, 170, 180, 190, 200, 250, 300, 350, 400, 450, or 500 amino acid residues correspond to 100, 110, 120, 130, 140, 150, 160, 170, 180, 190, 200, 250, 300, 350, 400, 450, or 500 consecutive amino acid residues of IL-2 or IL-15, respectively. In embodiments, the cytokine domain is 133 amino acid residues in length. In further embodiments, the 133 amino acid residues correspond to 133 consecutive amino acid residues of IL-2.

In embodiments, the cytokine domain includes a third receptor binding site, and the occlusion domain is positioned to sterically hinder binding of the third receptor binding site to the cognate receptor.

The term “occlusion domain” as used herein refers to a protein domain which sterically hinders binding of a receptor binding site to its cognate receptor. An occlusion domain as provided herein is sufficient in size, dimension or volume to create steric hindrance, thereby significantly decreasing (e.g., inhibiting or preventing) the ability of a receptor binding site to bind to its cognate receptor. The occlusion domain may be any molecule capable of hindering the interaction between a cytokine domain and its cognate receptor and includes without limitation an antibody domain, a Fab domain, a nanobody, a protein L domain, or a CD16 domain.

In embodiments, the occlusion domain is a protein domain. In embodiments, the occlusion domain is an antibody domain. In embodiments, the antibody domain is an Fc domain. In some embodiments, the Fc domain comprises an amino acid sequence selected from the group consisting of SEQ ID NOs: 3, 88, 89, 91, 93, 103, and 107. In some embodiments, the Fc domain comprises an amino acid sequence that has at least 90%, 91%, 92%, 93%, 94%, 95%, 96%, 97%, 98%, or 99% amino acid sequence identity to an amino acid sequence selected from the group consisting of SEQ ID NOs: 3, 88, 89, 91, 93, 103, and 107 across the whole sequence or a portion of the sequence (e.g. a 50, 75, or 100 continuous amino acid portion). In embodiments, the Fc domain includes a constant heavy chain 2 (CH2) domain or a constant heavy chain 3 (CH3) domain. In embodiments, the Fc domain is a CH2 domain. In some embodiments, the CH2 domain comprises the amino acid sequence of SEQ ID NO: 3. In some embodiments, the CH2 domain comprises an amino acid sequence that has at least 90%, 91%, 92%, 93%, 94%, 95%, 96%, 97%, 98%, or 99% amino acid sequence identity to the amino acid sequence of SEQ ID NO: 3 across the whole sequence or a portion of the sequence (e.g. a 50, 75, or 100 continuous amino acid portion).

In some embodiments, the CH2 domain comprises the amino acid sequence of SEQ ID NO: 88. In some embodiments, the CH2 domain comprises an amino acid sequence that has at least 90%, 91%, 92%, 93%, 94%, 95%, 96%, 97%, 98%, or 99% amino acid sequence identity to the amino acid sequence of SEQ ID NO: 88 across the whole sequence or a portion of the sequence (e.g. a 50, 75, or 100 continuous amino acid portion). In some embodiments, the CH2 domain comprises the amino acid sequence of SEQ ID NO: 89. In some embodiments, the CH2 domain comprises an amino acid sequence that has at least 90%, 91%, 92%, 93%, 94%, 95%, 96%, 97%, 98%, or 99% amino acid sequence identity to the amino acid sequence of SEQ ID NO: 89 across the whole sequence or a portion of the sequence (e.g. a 50, 75, or 100 continuous amino acid portion). In some embodiments, the CH2 domain comprises the amino acid sequence of SEQ ID NO: 91. In some embodiments, the CH2 domain comprises an amino acid sequence that has at least 90%, 91%, 92%, 93%, 94%, 95%, 96%, 97%, 98%, or 99% amino acid sequence identity to the amino acid sequence of SEQ ID NO: 91 across the whole sequence or a portion of the sequence (e.g. a 50, 75, or 100 continuous amino acid portion). In some embodiments, the CH2 domain comprises the amino acid sequence of SEQ ID NO: 93. In some embodiments, the CH2 domain comprises an amino acid sequence that has at least 90%, 91%, 92%, 93%, 94%, 95%, 96%, 97%, 98%, or 99% amino acid sequence identity to the amino acid sequence of SEQ ID NO: 93 across the whole sequence or a portion of the sequence (e.g. a 50, 75, or 100 continuous amino acid portion). In some embodiments, the CH2 domain comprises the amino acid sequence of SEQ ID NO: 103. In some embodiments, the CH2 domain comprises an amino acid sequence that has at least 90%, 91%, 92%, 93%, 94%, 95%, 96%, 97%, 98%, or 99% amino acid sequence identity to the amino acid sequence of SEQ ID NO: 103 across the whole sequence or a portion of the sequence (e.g. a 50, 75, or 100 continuous amino acid portion). In some embodiments, the CH2 domain comprises the amino acid sequence of SEQ ID NO: 107. In some embodiments, the CH2 domain comprises an amino acid sequence that has at least 90%, 91%, 92%, 93%, 94%, 95%, 96%, 97%, 98%, or 99% amino acid sequence identity to the amino acid sequence of SEQ ID NO: 107 across the whole sequence or a portion of the sequence (e.g. a 50, 75, or 100 continuous amino acid portion).

In some embodiments, the Fc domain is a truncated form of a CH2 domain. In embodiments, the Fc domain includes a CH2 domain and a CH3 domain. In embodiments, the Fc domain is a CH2 domain covalently linked to a CH3 domain. In embodiments, the CH2 domain is an IgG1 CH2 domain. In embodiments, the heavy chain constant (CH) domain (CH2 or CH3 domain) is the constant region of the heavy chain of an antibody or fragment thereof. In embodiments, the light chain constant (CL) domain is the constant region of the light chain of an antibody or fragment thereof. In embodiments, the heavy chain constant (CH) domain is the constant region of a Fab. In embodiments, the light chain constant (CL) domain is the constant region of the light chain of a Fab. In embodiments, the heavy chain constant (CH) domain is the constant region of a F(ab)′2 dimer. In embodiments, the light chain constant (CL) domain is the constant region of the light chain of a F(ab)′2 dimer.

In embodiments, the occlusion domain is a Fab domain. In some embodiments, the Fab domain comprises the amino acid sequence of SEQ ID NO: 102. In some embodiments, the Fab domain comprises an amino acid sequence that has at least 90%, 91%, 92%, 93%, 94%, 95%, 96%, 97%, 98%, or 99% amino acid sequence identity to the amino acid sequence of SEQ ID NO: 102 across the whole sequence or a portion of the sequence (e.g. a 50, 75, 100, 125, 150, 175, or 200 continuous amino acid portion). In embodiments, the Fab domain includes a variable light chain domain and a constant light chain domain. In embodiments, the Fab domain is a variable light chain domain bound to a constant light chain domain. In some embodiments, the Fab domain comprises a light chain comprising the amino acid sequence of SEQ ID NO: 51. In some embodiments, the Fab domain comprises a light chain comprising an amino acid sequence that has at least 90%, 91%, 92%, 93%, 94%, 95%, 96%, 97%, 98%, or 99% amino acid sequence identity to the amino acid sequence of SEQ ID NO: 51 across the whole sequence or a portion of the sequence (e.g. a 50, 75, 100, 125, 150, 175, or 200 continuous amino acid portion). In embodiments, the Fab domain includes a variable heavy chain domain and a constant heavy chain domain. In embodiments, the Fab domain is a variable heavy chain domain bound to a constant heavy chain domain. In some embodiments, the Fab domain comprises a heavy chain comprising the amino acid sequence of SEQ ID NO: 52. In some embodiments, the Fab domain comprises a heavy chain comprising an amino acid sequence that has at least 90%, 91%, 92%, 93%, 94%, 95%, 96%, 97%, 98%, or 99% amino acid sequence identity to the amino acid sequence of SEQ ID NO: 52 across the whole sequence or a portion of the sequence (e.g. a 50, 75, 100, 125, 150, 175, or 200 continuous amino acid portion). In some embodiments, the Fab domain is or comprises a heavy chain or a light chain of trastuzumab, or a portion or variant thereof.

A “light chain variable (VL) domain” as provided herein refers to the variable region of the light chain of an antibody, an antibody variant or fragment thereof. Likewise, the “heavy chain variable (VH) domain” as provided herein refers to the variable region of the heavy chain of an antibody, an antibody variant or fragment thereof.

In embodiments, the occlusion domain is a nanobody domain. In some embodiments, the nanobody domain comprises the amino acid sequence of SEQ ID NO: 95 or 97. In some embodiments, the nanobody domain comprises an amino acid sequence that has at least 90%, 91%, 92%, 93%, 94%, 95%, 96%, 97%, 98%, or 99% amino acid sequence identity to the amino acid sequence of SEQ ID NO: 95 or 97, across the whole sequence or a portion of the sequence (e.g. a 50, 75, or 100 continuous amino acid portion). In embodiments, the nanobody domain is a variable heavy chain domain. In embodiments, the nanobody domain is a variable light chain domain. In embodiments, the occlusion domain is a human serum albumin (HSA) moiety. In embodiments, the occlusion domain is a lipocalin moiety. In some embodiments, the occlusion domain is a protein L domain. In some embodiments, the protein L domain comprises the amino acid sequence of SEQ ID NO: 96, or comprises an amino acid sequence that has at least 90%, 91%, 92%, 93%, 94%, 95%, 96%, 97%, 98%, or 99% amino acid sequence identity to the amino acid sequence of SEQ ID NO: 96, across the whole sequence or a portion of the sequence (e.g. a 40, 50, or 60 continuous amino acid portion). In some embodiments, the occlusion domain is a CD16 domain. In some embodiments, the CD16 domain comprises the amino acid sequence of SEQ ID NO: 98, or comprises an amino acid sequence that has at least 90%, 91%, 92%, 93%, 94%, 95%, 96%, 97%, 98%, or 99% amino acid sequence identity to the amino acid sequence of SEQ ID NO: 98, across the whole sequence or a portion of the sequence (e.g. a 50, 75, 100, 125, 150, or 175 continuous amino acid portion). In some embodiments, the occlusion domain is an IgG binding protein that binds to, or otherwise exhibits affinity for, an IgG. In some embodiments, the IgG binding protein is a nanobody, a HSA moiety, a CD16 domain, or a protein L domain.

In embodiments, the first receptor binding site is an IL-2Rα binding site. The first receptor binding site may be non-covalently bound to the first receptor domain (e.g., IL-2 domain). Thus, in embodiments, the first receptor domain binds non-covalently to the first receptor binding site. In some embodiments, the first receptor domain is an IL-2Rα domain. In some embodiments, the first receptor domain comprises the amino acid sequence of SEQ ID NO: 5. In some embodiments, the first receptor domain comprises an amino acid sequence that has at least 90%, 91%, 92%, 93%, 94%, 95%, 96%, 97%, 98%, or 99% amino acid sequence identity to the amino acid sequence of SEQ ID NO: 5 across the whole sequence or a portion of the sequence (e.g. a 50, 75, 100, 125, or 150 continuous amino acid portion). In some embodiments, the first receptor domain comprises the amino acid sequence of SEQ ID NO: 50. In some embodiments, the first receptor domain comprises an amino acid sequence that has at least 90%, 91%, 92%, 93%, 94%, 95%, 96%, 97%, 98%, or 99% amino acid sequence identity to the amino acid sequence of SEQ ID NO: 50 across the whole sequence or a portion of the sequence (e.g. a 50, 75, 100, 125, or 150 continuous amino acid portion). In some embodiments, the first receptor domain comprises the amino acid sequence of SEQ ID NO: 59. In some embodiments, the first receptor domain comprises an amino acid sequence that has at least 90%, 91%, 92%, 93%, 94%, 95%, 96%, 97%, 98%, or 99% amino acid sequence identity to the amino acid sequence of SEQ ID NO: 59 across the whole sequence or a portion of the sequence (e.g. a 50, 75, 100, 125, or 150 continuous amino acid portion). In some embodiments, the first receptor domain comprises the amino acid sequence of SEQ ID NO: 90. In some embodiments, the first receptor domain comprises an amino acid sequence that has at least 90%, 91%, 92%, 93%, 94%, 95%, 96%, 97%, 98%, or 99% amino acid sequence identity to the amino acid sequence of SEQ ID NO: 90 across the whole sequence or a portion of the sequence (e.g. a 50, 75, 100, 125, or 150 continuous amino acid portion). In some embodiments, the first receptor domain comprises the amino acid sequence of SEQ ID NO: 100 or 101. In some embodiments, the first receptor domain comprises an amino acid sequence that has at least 90%, 91%, 92%, 93%, 94%, 95%, 96%, 97%, 98%, or 99% amino acid sequence identity to the amino acid sequence of SEQ ID NO: 100 or 101 across the whole sequence or a portion of the sequence (e.g. a 50, 75, 100, 125, or 150 continuous amino acid portion). In some embodiments, the first receptor domain comprises the amino acid sequence of SEQ ID NO: 105. In some embodiments, the first receptor domain comprises an amino acid sequence that has at least 90%, 91%, 92%, 93%, 94%, 95%, 96%, 97%, 98%, or 99% amino acid sequence identity to the amino acid sequence of SEQ ID NO: 105 across the whole sequence or a portion of the sequence (e.g. a 50, 75, 100, 125, or 150 continuous amino acid portion). In embodiments, the second receptor binding site is an IL-2Rγ binding site. In embodiments, the second receptor binding site is an IL-2Rβ binding site. In embodiments, the third receptor binding site is an IL-2Rγ binding site. In embodiments, the third receptor binding site is an IL-2Rβ binding site.

In embodiments, the cognate receptor includes an IL-2 receptor β or fragment thereof or an IL-2 receptor γ or fragment thereof. In embodiments, the cognate receptor includes an IL-2 receptor β or fragment thereof. In embodiments, the cognate receptor includes an IL-2 receptor γ or fragment thereof. In embodiments, the cognate receptor includes an IL-2 receptor β or fragment thereof and an IL-2 receptor γ or fragment thereof. In embodiments, the cognate receptor includes an IL-2 receptor α or fragment thereof, an IL-2 receptor β or fragment thereof or an IL-2 receptor γ or fragment thereof. In embodiments, the cognate receptor includes an IL-2 receptor α or fragment thereof, an IL-2 receptor β or fragment thereof and an IL-2 receptor γ or fragment thereof.

In embodiments, the first receptor binding site is an IL-15Rα binding site. The first receptor binding site may be non-covalently bound to the first receptor domain (e.g., IL-15 domain). Thus, in embodiments, the first receptor domain binds non-covalently to the first receptor binding site. In some embodiments, the first receptor domain is an IL-15Rα domain. In some embodiments, the first receptor domain comprises the amino acid sequence of SEQ ID NO: 94. In some embodiments, the first receptor domain comprises an amino acid sequence that has at least 90%, 91%, 92%, 93%, 94%, 95%, 96%, 97%, 98%, or 99% amino acid sequence identity to the amino acid sequence of SEQ ID NO: 94 across the whole sequence or a portion of the sequence (e.g. a 50, 75, 100, 125, or 150 continuous amino acid portion).

A “chemical linker,” as provided herein, is a covalent linker, a non-covalent linker, a peptide or peptidyl linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof. Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

The chemical linker as provided herein (e.g., first or second chemical linker), may be —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein (e.g., first or second chemical linker), may be —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered,) heteroarylene.

In embodiments, the first chemical linker and the second chemical linker are independently a covalent linker or a non-covalent linker. In embodiments, the first chemical linker and the second chemical linker are independently a peptidyl linker. In embodiments, the first chemical linker and the second chemical linker are independently a cleavable linker. In embodiments, the cleavable linker is a protease cleavable linker. Thus, in embodiments, the first chemical linker and the second chemical linker are independently an enzymatically cleavable linker. In embodiments, the first chemical linker and the second chemical linker are independently a protease cleavable linker. In embodiments, the cleavable linker is a tumor-associated protease cleavable linker.

In embodiments, the first chemical linker and the second chemical linker are independently a cleavable linker including a protease cleavage site. In some embodiments, the first chemical linker is a cleavable linker that includes a protease cleavage site. In some embodiments, the second chemical linker is a cleavable linker that includes a protease cleavage site. A “cleavable linker” refers to a linker including an element (e.g., peptide sequence) that is labile to cleavage upon suitable manipulation (e.g., protease activity). Accordingly, a cleavable linker may comprise any of a number of chemical entities, including amino acids, nucleic acids, or small molecules, among others. A cleavable linker may be cleaved by, for instance, chemical, enzymatic, or physical means. Non-limiting examples of labile elements included in cleavable linkers include protease cleavage sites, nucleic acid sequences cleaved by nucleases, photolabile, acid-labile, or base-labile functional groups.

A “cleavage site” as used herein, refers to a recognizable site for cleavage of a portion of a linker described herein. Thus, a cleavage site may be found in the sequence of a cleavable peptide linker as described herein, including embodiments thereof. In embodiments, the cleavage site is an amino acid sequence that is recognized and cleaved by a cleaving agent (e.g., a peptidyl sequence). In some embodiments, the cleavage site comprises the amino acid sequence VPLSLY (SEQ ID NO: 29). In some embodiments, the cleavage site comprises the amino acid sequence SPLAQAVRSS (SEQ ID NO: 64). Exemplary cleaving agents include proteins, enzymes, DNAzymes, RNAzymes, metals, acids, and bases. In embodiments, the protease cleavage site is a matrix metalloprotease (MMP) cleavage site, a disintegrin and metalloprotease domain-containing (ADAM) metalloprotease cleavage site, a prostate specific antigen (PSA) protease cleavage site, a urokinase-type plasminogen activator (uPA) protease cleavage site, a membrane type serine protease 1 (MT-SP1) protease cleavage site or a legumain protease cleavage site. In embodiments, the matrix metalloprotease (MMP) cleavage site is a MMP 9 cleavage site, a MMP 13 cleavage site or a MMP 2 cleavage site. In embodiments, the disintegrin and metalloprotease domain-containing (ADAM) metalloprotease cleavage site is an ADAM 9 metalloprotease cleavage site, an ADAM 10 metalloprotease cleavage site or an ADAM 17 metalloprotease cleavage site.

Further exemplary cleavage sites include the cleavage site of ABHD12, ADAM12, ABHD12B, ABHD13, ABHD17A, ADAM19, ADAM20, ADAM21, ADAM28, ADAM30, ADAM33, ADAMS, ABHD17A, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ABHD17B, ADAMTS5, ADAMTS6, ADAMTS7, ADAMTS8, ADAMTS9, ADAMTSL1, ADAMTSL2, ADAMTSL3, ABHD17C, ADAMTSL5, ASTL, BMP1, CELA1, CELA2A, CELA2B, CELA3A, CELA3B, ADAM10, ADAM15, ADAM17, ADAMS, ADAMTS4, CTSE, CTSF, ADAMTSL4, CMA1, CTRB1, CTRC, CTSO, CTR1, CTSA, CTSW, CTSB, CTSC, CTSD, ESP1, CTSG, CTSH, GZMA, GZMB, GZMH, CTSK, GZMM, CTSL, CTSS, CTSV, CTSZ, HTRA4, KLK10, KLK11, KLK13, KLK14, KLK2, KLK4, DPP4, KLK6, KLK7, KLKB1, ECE1, ECE2, ECEL1, MASP2, MEP1A, MEP1B, ELANE, FAP, GZMA, MMP11, GZMK, HGFAC, HPN, HTRA1, MMP11, MMP16, MMP17, MMP19, HTRA2, MMP20, MMP21, HTRA3, HTRA4, KEL, MMP23B, MMP24, MMP25, MMP26, MMP27, MMP28, KLK5, MMP3, MMP7, MMP8, MMP9, LGMN, LNPEP, MASP1, PAPPA, PAPPA2, PCSK1, NAPSA, PCSK5, PCSK6, MME, MMP1, MMP10, PLAT, PLAU, PLG, PRSS1, PRSS12, PRSS2, PRSS21, PRSS3, PRSS33, PRSS4, PRSS55, PRSS57, MMP12, PRSS8, PRSS9, PRTN3, MMP13, MMP14, ST14, TMPRSS10, TMPRSS11A, TMPRSS11D, TMPRSS11E, TMPRSS11F, TMPRSS12, TMPRSS13, MMP15, TMPRSS15, MMP2, TMPRSS2, TMPRSS3, TMPRSS4, TMPRSS5, TMPRSS6, TMPRSS7, TMPRSS9, NRDC, OVCH1, PAMR1, PCSK3, PHEX, TINAG, TPSAB1, TPSD1, or TPSG1.

In embodiments, the cleavable linker is a tumor-associated cleavable linker. In embodiments, the chemical linker is a protease cleavable linker. In embodiments, the chemical linker is a tumor-associated protease cleavable linker. A “tumor-associated protease cleavable linker” as provided herein is an amino acid sequence recognized by a protease, whose expression is specific for a tumor cell or tumor cell environment thereof.

“Proteases” (or “proteinases”, “peptidases”, or “proteolytic” enzymes) generally refer to a class of enzymes that cleave peptide bonds between amino acids of proteins. Because proteases use a molecule of water to effect hydrolysis of peptide bonds, these enzymes can also be classified as hydrolases. Six classes of proteases are presently known: serine proteases, threonine proteases, cysteine proteases, aspartic acid proteases, metalloproteases, and glutamic acid proteases (see, e.g., Barrett A. J. et al. The Handbook of Proteolytic Enzymes, 2nd ed. Academic Press, 2003). A “tumor-associated protease” refers to a class of enzymes whose expression is specific for a tumor cell or tumor cell environment thereof. A tumor-associated protease may be expressed by a tumor and/or non-tumor cells. Non-limiting examples of proteases contemplated for the invention provided herein including embodiments thereof include ABHD12, ADAM12, ABHD12B, ABHD13, ABHD17A, ADAM19, ADAM20, ADAM21, ADAM28, ADAM30, ADAM33, ADAMS, ABHD17A, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ABHD17B, ADAMTS5, ADAMTS6, ADAMTS7, ADAMTS8, ADAMTS9, ADAMTSL1, ADAMTSL2, ADAMTSL3, ABHD17C, ADAMTSL5, ASTL, BMP1, CELA1, CELA2A, CELA2B, CELA3A, CELA3B, ADAM10, ADAM15, ADAM17, ADAMS, ADAMTS4, CTSE, CTSF, ADAMTSL4, CMA1, CTRB1, CTRC, CTSO, CTR1, CTSA, CTSW, CTSB, CTSC, CTSD, ESP1, CTSG, CTSH, GZMA, GZMB, GZMH, CTSK, GZMM, CTSL, CTSS, CTSV, CTSZ, HTRA4, KLK10, KLK11, KLK13, KLK14, KLK2, KLK4, DPP4, KLK6, KLK7, KLKB1, ECE1, ECE2, ECEL1, MASP2, MEP1A, MEP1B, ELANE, FAP, GZMA, MMP11, GZMK, HGFAC, HPN, HTRA1, MMP11, MMP16, MMP17, MMP19, HTRA2, MMP20, MMP21, HTRA3, HTRA4, KEL, MMP23B, MMP24, MMP25, MMP26, MMP27, MMP28, KLK5, MMP3, MMP7, MMP8, MMP9, LGMN, LNPEP, MASP1, PAPPA, PAPPA2, PCSK1, NAPSA, PCSK5, PCSK6, MME, MMP1, MMP10, PLAT, PLAU, PLG, PRSS1, PRSS12, PRSS2, PRSS21, PRSS3, PRSS33, PRSS4, PRSS55, PRSS57, MMP12, PRSS8, PRSS9, PRTN3, MMP13, MMP14, ST14, TMPRSS10, TMPRSS11A, TMPRSS11D, TMPRSS11E, TMPRSS11F, TMPRSS12, TMPRSS13, MMP15, TMPRSS15, MMP2, TMPRSS2, TMPRSS3, TMPRSS4, TMPRSS5, TMPRSS6, TMPRSS7, TMPRSS9, NRDC, OVCH1, PAMR1, PCSK3, PHEX, TINAG, TPSAB1, TPSD1, and TPSG1.

The chemical linkers provided herein, including embodiments thereof, may have different lengths (e.g., include varying numbers of amino acid residues). In embodiments, the first chemical linker and the second chemical linker independently have a length of less than about 30 amino acid residues. In embodiments, the first chemical linker and the second chemical linker independently have a length of 0 to 30 amino acid residues. In embodiments, the first chemical linker and the second chemical linker independently have a length of 0 to 25 amino acid residues. In embodiments, the first chemical linker and the second chemical linker independently have a length of 0 to 20 amino acid residues. In embodiments, the first chemical linker and the second chemical linker independently have a length of 0 to 15 amino acid residues. In embodiments, the first chemical linker and the second chemical linker independently have a length of 0 to 10 amino acid residues. In embodiments, the first chemical linker and the second chemical linker independently have a length of 0 to 9 amino acid residues. In embodiments, the first chemical linker and the second chemical linker independently have a length of 0 to 8 amino acid residues. In embodiments, the first chemical linker and the second chemical linker independently have a length of 0 to 7 amino acid residues. In embodiments, the first chemical linker and the second chemical linker independently have a length of 0 to 6 amino acid residues. In embodiments, the first chemical linker and the second chemical linker independently have a length of 0 to 5 amino acid residues. In embodiments, the first chemical linker and the second chemical linker independently have a length of 0 to 4 amino acid residues. In embodiments, the first chemical linker and the second chemical linker independently have a length of 0 to 3 amino acid residues. In embodiments, the first chemical linker and the second chemical linker independently have a length of 0 to 2 amino acid residues.

In embodiments, the first chemical linker and the second chemical linker independently have a length of 1 to 30 amino acid residues. In embodiments, the first chemical linker and the second chemical linker independently have a length of 1 to 25 amino acid residues. In embodiments, the first chemical linker and the second chemical linker independently have a length of 1 to 20 amino acid residues. In embodiments, the first chemical linker and the second chemical linker independently have a length of 1 to 15 amino acid residues. In embodiments, the first chemical linker and the second chemical linker independently have a length of 1 to 10 amino acid residues. In embodiments, the first chemical linker and the second chemical linker independently have a length of 1 to 9 amino acid residues. In embodiments, the first chemical linker and the second chemical linker independently have a length of 1 to 8 amino acid residues. In embodiments, the first chemical linker and the second chemical linker independently have a length of 1 to 7 amino acid residues. In embodiments, the first chemical linker and the second chemical linker independently have a length of 1 to 6 amino acid residues. In embodiments, the first chemical linker and the second chemical linker independently have a length of 1 to 5 amino acid residues. In embodiments, the first chemical linker and the second chemical linker independently have a length of 1 to 4 amino acid residues. In embodiments, the first chemical linker and the second chemical linker independently have a length of 1 to 3 amino acid residues.

In embodiments, the first chemical linker and the second chemical linker independently have a length of 2 to 30 amino acid residues. In embodiments, the first chemical linker and the second chemical linker independently have a length of 2 to 25 amino acid residues. In embodiments, the first chemical linker and the second chemical linker independently have a length of 2 to 20 amino acid residues. In embodiments, the first chemical linker and the second chemical linker independently have a length of 2 to 15 amino acid residues. In embodiments, the first chemical linker and the second chemical linker independently have a length of 2 to 10 amino acid residues. In embodiments, the first chemical linker and the second chemical linker independently have a length of 2 to 9 amino acid residues. In embodiments, the first chemical linker and the second chemical linker independently have a length of 2 to 8 amino acid residues. In embodiments, the first chemical linker and the second chemical linker independently have a length of 2 to 7 amino acid residues. In embodiments, the first chemical linker and the second chemical linker independently have a length of 2 to 6 amino acid residues. In embodiments, the first chemical linker and the second chemical linker independently have a length of 2 to 5 amino acid residues. In embodiments, the first chemical linker and the second chemical linker independently have a length of 2 to 4 amino acid residues.

In embodiments, the first chemical linker and the second chemical linker independently have a length of 3 to 30 amino acid residues. In embodiments, the first chemical linker and the second chemical linker independently have a length of 3 to 25 amino acid residues. In embodiments, the first chemical linker and the second chemical linker independently have a length of 3 to 20 amino acid residues. In embodiments, the first chemical linker and the second chemical linker independently have a length of 3 to 15 amino acid residues. In embodiments, the first chemical linker and the second chemical linker independently have a length of 3 to 10 amino acid residues. In embodiments, the first chemical linker and the second chemical linker independently have a length of 3 to 9 amino acid residues. In embodiments, the first chemical linker and the second chemical linker independently have a length of 3 to 8 amino acid residues. In embodiments, the first chemical linker and the second chemical linker independently have a length of 3 to 7 amino acid residues. In embodiments, the first chemical linker and the second chemical linker independently have a length of 3 to 6 amino acid residues. In embodiments, the first chemical linker and the second chemical linker independently have a length of 3 to 5 amino acid residues.

In embodiments, the first chemical linker and the second chemical linker independently have a length of 4 to 30 amino acid residues. In embodiments, the first chemical linker and the second chemical linker independently have a length of 4 to 25 amino acid residues. In embodiments, the first chemical linker and the second chemical linker independently have a length of 4 to 20 amino acid residues. In embodiments, the first chemical linker and the second chemical linker independently have a length of 4 to 15 amino acid residues. In embodiments, the first chemical linker and the second chemical linker independently have a length of 4 to 10 amino acid residues. In embodiments, the first chemical linker and the second chemical linker independently have a length of 4 to 9 amino acid residues. In embodiments, the first chemical linker and the second chemical linker independently have a length of 4 to 8 amino acid residues. In embodiments, the first chemical linker and the second chemical linker independently have a length of 4 to 7 amino acid residues. In embodiments, the first chemical linker and the second chemical linker independently have a length of 4 to 6 amino acid residues.

In embodiments, the first chemical linker and the second chemical linker independently have a length of 5 to 30 amino acid residues. In embodiments, the first chemical linker and the second chemical linker independently have a length of 5 to 25 amino acid residues. In embodiments, the first chemical linker and the second chemical linker independently have a length of 5 to 20 amino acid residues. In embodiments, the first chemical linker and the second chemical linker independently have a length of 5 to 15 amino acid residues. In embodiments, the first chemical linker and the second chemical linker independently have a length of 5 to 10 amino acid residues. In embodiments, the first chemical linker and the second chemical linker independently have a length of 5 to 9 amino acid residues. In embodiments, the first chemical linker and the second chemical linker independently have a length of 5 to 8 amino acid residues. In embodiments, the first chemical linker and the second chemical linker independently have a length of 5 to 7 amino acid residues.

In embodiments, the first chemical linker and the second chemical linker independently have a length of 6 to 30 amino acid residues. In embodiments, the first chemical linker and the second chemical linker independently have a length of 6 to 25 amino acid residues. In embodiments, the first chemical linker and the second chemical linker independently have a length of 6 to 20 amino acid residues. In embodiments, the first chemical linker and the second chemical linker independently have a length of 6 to 15 amino acid residues. In embodiments, the first chemical linker and the second chemical linker independently have a length of 6 to 10 amino acid residues. In embodiments, the first chemical linker and the second chemical linker independently have a length of 6 to 9 amino acid residues. In embodiments, the first chemical linker and the second chemical linker independently have a length of 6 to 8 amino acid residues.

In embodiments, the first chemical linker and the second chemical linker independently have a length of 7 to 30 amino acid residues. In embodiments, the first chemical linker and the second chemical linker independently have a length of 7 to 25 amino acid residues. In embodiments, the first chemical linker and the second chemical linker independently have a length of 7 to 20 amino acid residues. In embodiments, the first chemical linker and the second chemical linker independently have a length of 7 to 15 amino acid residues. In embodiments, the first chemical linker and the second chemical linker independently have a length of 7 to 10 amino acid residues. In embodiments, the first chemical linker and the second chemical linker independently have a length of 7 to 9 amino acid residues.

In embodiments, the first chemical linker and the second chemical linker independently have a length of 8 to 30 amino acid residues. In embodiments, the first chemical linker and the second chemical linker independently have a length of 8 to 25 amino acid residues. In embodiments, the first chemical linker and the second chemical linker independently have a length of 8 to 20 amino acid residues. In embodiments, the first chemical linker and the second chemical linker independently have a length of 8 to 15 amino acid residues. In embodiments, the first chemical linker and the second chemical linker independently have a length of 8 to 10 amino acid residues. In embodiments, the first chemical linker and the second chemical linker independently have a length of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

In embodiments, the first chemical linker and the second chemical linker independently have a length of about 0 to about 30 amino acid residues. In embodiments, the first chemical linker and the second chemical linker independently have a length of about 0 to about 25 amino acid residues. In embodiments, the first chemical linker and the second chemical linker independently have a length of about 0 to about 20 amino acid residues. In embodiments, the first chemical linker and the second chemical linker independently have a length of about 0 to about 15 amino acid residues. In embodiments, the first chemical linker and the second chemical linker independently have a length of about 0 to about 10 amino acid residues. In embodiments, the first chemical linker and the second chemical linker independently have a length of about 0 to about 9 amino acid residues. In embodiments, the first chemical linker and the second chemical linker independently have a length of about 0 to about 8 amino acid residues. In embodiments, the first chemical linker and the second chemical linker independently have a length of about 0 to about 7 amino acid residues. In embodiments, the first chemical linker and the second chemical linker independently have a length of about 0 to about 6 amino acid residues. In embodiments, the first chemical linker and the second chemical linker independently have a length of about 0 to about 5 amino acid residues. In embodiments, the first chemical linker and the second chemical linker independently have a length of about 0 to about 4 amino acid residues. In embodiments, the first chemical linker and the second chemical linker independently have a length of about 0 to about 3 amino acid residues. In embodiments, the first chemical linker and the second chemical linker independently have a length of about 0 to about 2 amino acid residues.

In embodiments, the first chemical linker and the second chemical linker independently have a length of about 1 to about 30 amino acid residues. In embodiments, the first chemical linker and the second chemical linker independently have a length of about 1 to about 25 amino acid residues. In embodiments, the first chemical linker and the second chemical linker independently have a length of about 1 to about 20 amino acid residues. In embodiments, the first chemical linker and the second chemical linker independently have a length of about 1 to about 15 amino acid residues. In embodiments, the first chemical linker and the second chemical linker independently have a length of about 1 to about 10 amino acid residues. In embodiments, the first chemical linker and the second chemical linker independently have a length of about 1 to about 9 amino acid residues. In embodiments, the first chemical linker and the second chemical linker independently have a length of about 1 to about 8 amino acid residues. In embodiments, the first chemical linker and the second chemical linker independently have a length of about 1 to about 7 amino acid residues. In embodiments, the first chemical linker and the second chemical linker independently have a length of about 1 to about 6 amino acid residues. In embodiments, the first chemical linker and the second chemical linker independently have a length of about 1 to about 5 amino acid residues. In embodiments, the first chemical linker and the second chemical linker independently have a length of about 1 to about 4 amino acid residues. In embodiments, the first chemical linker and the second chemical linker independently have a length of about 1 to about 3 amino acid residues.

In embodiments, the first chemical linker and the second chemical linker independently have a length of about 2 to about 30 amino acid residues. In embodiments, the first chemical linker and the second chemical linker independently have a length of about 2 to about 25 amino acid residues. In embodiments, the first chemical linker and the second chemical linker independently have a length of about 2 to about 20 amino acid residues. In embodiments, the first chemical linker and the second chemical linker independently have a length of about 2 to about 15 amino acid residues. In embodiments, the first chemical linker and the second chemical linker independently have a length of about 2 to about 10 amino acid residues. In embodiments, the first chemical linker and the second chemical linker independently have a length of about 2 to about 9 amino acid residues. In embodiments, the first chemical linker and the second chemical linker independently have a length of about 2 to about 8 amino acid residues. In embodiments, the first chemical linker and the second chemical linker independently have a length of about 2 to about 7 amino acid residues. In embodiments, the first chemical linker and the second chemical linker independently have a length of about 2 to about 6 amino acid residues. In embodiments, the first chemical linker and the second chemical linker independently have a length of about 2 to about 5 amino acid residues. In embodiments, the first chemical linker and the second chemical linker independently have a length of about 2 to about 4 amino acid residues.

In embodiments, the first chemical linker and the second chemical linker independently have a length of about 3 to about 30 amino acid residues. In embodiments, the first chemical linker and the second chemical linker independently have a length of about 3 to about 25 amino acid residues. In embodiments, the first chemical linker and the second chemical linker independently have a length of about 3 to about 20 amino acid residues. In embodiments, the first chemical linker and the second chemical linker independently have a length of about 3 to about 15 amino acid residues. In embodiments, the first chemical linker and the second chemical linker independently have a length of about 3 to about 10 amino acid residues. In embodiments, the first chemical linker and the second chemical linker independently have a length of about 3 to about 9 amino acid residues. In embodiments, the first chemical linker and the second chemical linker independently have a length of about 3 to about 8 amino acid residues. In embodiments, the first chemical linker and the second chemical linker independently have a length of about 3 to about 7 amino acid residues. In embodiments, the first chemical linker and the second chemical linker independently have a length of about 3 to about 6 amino acid residues. In embodiments, the first chemical linker and the second chemical linker independently have a length of about 3 to about 5 amino acid residues.

In embodiments, the first chemical linker and the second chemical linker independently have a length of about 4 to about 30 amino acid residues. In embodiments, the first chemical linker and the second chemical linker independently have a length of about 4 to about 25 amino acid residues. In embodiments, the first chemical linker and the second chemical linker independently have a length of about 4 to about 20 amino acid residues. In embodiments, the first chemical linker and the second chemical linker independently have a length of about 4 to about 15 amino acid residues. In embodiments, the first chemical linker and the second chemical linker independently have a length of about 4 to about 10 amino acid residues. In embodiments, the first chemical linker and the second chemical linker independently have a length of about 4 to about 9 amino acid residues. In embodiments, the first chemical linker and the second chemical linker independently have a length of about 4 to about 8 amino acid residues. In embodiments, the first chemical linker and the second chemical linker independently have a length of about 4 to about 7 amino acid residues. In embodiments, the first chemical linker and the second chemical linker independently have a length of about 4 to about 6 amino acid residues.

In embodiments, the first chemical linker and the second chemical linker independently have a length of about 5 to about 30 amino acid residues. In embodiments, the first chemical linker and the second chemical linker independently have a length of about 5 to about 25 amino acid residues. In embodiments, the first chemical linker and the second chemical linker independently have a length of about 5 to about 20 amino acid residues. In embodiments, the first chemical linker and the second chemical linker independently have a length of about 5 to about 15 amino acid residues. In embodiments, the first chemical linker and the second chemical linker independently have a length of about 5 to about 10 amino acid residues. In embodiments, the first chemical linker and the second chemical linker independently have a length of about 5 to about 9 amino acid residues. In embodiments, the first chemical linker and the second chemical linker independently have a length of about 5 to about 8 amino acid residues. In embodiments, the first chemical linker and the second chemical linker independently have a length of about 5 to about 7 amino acid residues.

In embodiments, the first chemical linker and the second chemical linker independently have a length of about 6 to about 30 amino acid residues. In embodiments, the first chemical linker and the second chemical linker independently have a length of about 6 to about 25 amino acid residues. In embodiments, the first chemical linker and the second chemical linker independently have a length of about 6 to about 20 amino acid residues. In embodiments, the first chemical linker and the second chemical linker independently have a length of about 6 to about 15 amino acid residues. In embodiments, the first chemical linker and the second chemical linker independently have a length of about 6 to about 10 amino acid residues. In embodiments, the first chemical linker and the second chemical linker independently have a length of about 6 to about 9 amino acid residues. In embodiments, the first chemical linker and the second chemical linker independently have a length of about 6 to about 8 amino acid residues.

In embodiments, the first chemical linker and the second chemical linker independently have a length of about 7 to about 30 amino acid residues. In embodiments, the first chemical linker and the second chemical linker independently have a length of about 7 to about 25 amino acid residues. In embodiments, the first chemical linker and the second chemical linker independently have a length of about 7 to about 20 amino acid residues. In embodiments, the first chemical linker and the second chemical linker independently have a length of about 7 to about 15 amino acid residues. In embodiments, the first chemical linker and the second chemical linker independently have a length of about 7 to about 10 amino acid residues. In embodiments, the first chemical linker and the second chemical linker independently have a length of about 7 to about 9 amino acid residues.

In embodiments, the first chemical linker and the second chemical linker independently have a length of about 8 to about 30 amino acid residues. In embodiments, the first chemical linker and the second chemical linker independently have a length of about 8 to about 25 amino acid residues. In embodiments, the first chemical linker and the second chemical linker independently have a length of about 8 to about 20 amino acid residues. In embodiments, the first chemical linker and the second chemical linker independently have a length of about 8 to about 15 amino acid residues. In embodiments, the first chemical linker and the second chemical linker independently have a length of about 8 to about 10 amino acid residues. In embodiments, the first chemical linker and the second chemical linker independently have a length of about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

In embodiments, the first chemical linker and the second chemical linker independently have a length of 5 or 6 amino acid residues. In embodiments, the first chemical linker and the second chemical linker independently have a length of 5 amino acid residues. In embodiments, the first chemical linker and the second chemical linker independently have a length of 6 amino acid residues.

In some embodiments, the first chemical linker comprises an amino acid sequence selected from the group consisting of SEQ ID NOs: 2, 4, 9-25, 34-37, 61-63, and 104. In some embodiments, the second chemical linker comprises an amino acid sequence selected from the group consisting of SEQ ID NOs: 2, 4, 9-25, 34-37, 61-63, and 104. In some embodiments, the first chemical linker comprises an amino acid sequence selected from the group consisting of SEQ ID NOs: 2, 4, 9-25, 34-37, 61-63, and 104, and the second chemical linker comprises an amino acid sequence selected from the group consisting of SEQ ID NOs: 2, 4, 9-25, 34-37, 61-63, and 104. In some embodiments, the first chemical linker comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 2, 4, 9-25, 34-37, 61-63, and 104 across the whole sequence or a portion of the sequence. In some embodiments, the second chemical linker comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 2, 4, 9-25, 34-37, 61-63, and 104 across the whole sequence or a portion of the sequence. In some embodiments, the first chemical linker comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 2, 4, 9-25, 34-37, 61-63, and 104 across the whole sequence or a portion of the sequence, and the second chemical linker comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 2, 4, 9-25, 34-37, 61-63, and 104 across the whole sequence or a portion of the sequence. In some embodiments, the first chemical linker comprises an amino acid sequence selected from the group consisting of SEQ ID NOs: 2, 19, 22, 25, 34, 36, 37, 61, and 62 and the second chemical linker comprises an amino acid sequence selected from the group consisting of SEQ ID NOs: 9, 11, 12, 14, 15, 17, 18, 20, 21, 24, 35, and 63. In some embodiments comprising a third chemical linker, the third chemical linker comprises an amino acid sequence selected from the group consisting of SEQ ID NOs: 2, 4, 9-25, 34-37, 61-63, and 104, or comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 2, 4, 9-25, 34-37, 61-63, and 104 across the whole sequence or a portion of the sequence. In some embodiments comprising a third chemical linker, the third chemical linker comprises the amino acid sequence of SEQ ID NO: 104.

In some embodiments, the first chemical linker includes a cleavage site comprising the amino acid sequence of SEQ ID NO: 29. In some embodiments, the second chemical linker includes a cleavage site comprising the amino acid sequence of SEQ ID NO: 29. In some embodiments, the first chemical linker includes a cleavage site comprising the amino acid sequence of SEQ ID NO: 64. In some embodiments, the second chemical linker includes a cleavage site comprising the amino acid sequence of SEQ ID NO: 64.

In some embodiments, the recombinant cytokine receptor binding protein further comprises an Fc binding peptide. In some embodiments, the Fc binding peptide comprises the amino acid sequence DCAWHLGELVWCT (SEQ ID NO: 31). In some embodiments, the Fc binding peptide comprises the amino acid sequence of SEQ ID NO: 31 and further comprises one or more amino acid residues linked to the N-terminus and/or the C-terminus of the amino acid sequence of SEQ ID NO: 31. In some embodiments, the Fc binding peptide comprises the amino acid sequence of SEQ ID NO: 32 or SEQ ID NO: 33. In some embodiments, the Fc binding peptide is comprised within a first chemical linker. In some embodiments, the Fc binding peptide is comprised within a second chemical linker. In some embodiments, a first chemical linker that includes an Fc binding peptide comprises an amino acid sequence selected from the group consisting of SEQ ID NOs: 34-37. In some embodiments, the first chemical linker that includes an Fc binding domain comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 34-37. In some embodiments, a second chemical linker that includes an Fc binding peptide comprises an amino acid sequence selected from the group consisting of SEQ ID NOs: 34-37. In some embodiments, the second chemical linker that includes an Fc binding domain comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 34-37. In some embodiments, the Fc binding peptide is bound to the N-terminus of the occlusion domain. In some embodiments, the Fc binding peptide is bound to the C-terminus of the occlusion domain. In some embodiments, the Fc binding peptide is bound to the N-terminus of the Fc domain. In some embodiments, the Fc binding peptide is bound to the C-terminus of the Fc domain. In some embodiments, the Fc binding peptide is bound to the N-terminus of the CH2 domain. In some embodiments, the Fc binding peptide is bound to the C-terminus of the CH2 domain. In some embodiments, the Fc binding peptide is bound to the N-terminus of the cytokine domain. In some embodiments, the Fc binding peptide is bound to the C-terminus of the cytokine domain.

In some embodiments, the recombinant cytokine receptor binding protein comprises a histidine tag. In some embodiments, the histidine tag includes the amino acid sequence HHHHHH (SEQ ID NO: 58). In some embodiments, the histidine tag is linked to linker or spacer domain and comprises the amino acid sequence of SEQ ID NO: 57.

In still another aspect is provided a recombinant cytokine receptor binding protein including: (i) an IL-2 domain including an IL-2 receptor α binding site, an IL-2 receptor β binding site and an IL-2 receptor γ binding site; (ii) an IL-2 receptor α domain that specifically binds to the IL-2 receptor α binding site; and (iii) an Fc domain positioned to sterically hinder binding of the IL-2 receptor β binding site to an IL-2 receptor β domain. The C-terminus of the IL-2 domain is bound to the N-terminus of the Fc domain through a cleavable linker; and the C-terminus of the Fc domain is bound to the N-terminus of the IL2 receptor α domain through a chemical linker.

In embodiments, the Fc domain is positioned to sterically hinder binding of the IL-2 receptor γ binding site to an IL-2 receptor γ domain.

In embodiments, the cytokine domain includes the sequence of SEQ ID NO:1. In embodiments, the cytokine domain is the sequence of SEQ ID NO:1. In embodiments, the first chemical linker includes the sequence of SEQ ID NO:2. In embodiments, the first chemical linker is the sequence of SEQ ID NO:2. In embodiments, the occlusion domain includes the sequence of SEQ ID NO:3. In embodiments, the occlusion domain is the sequence of SEQ ID NO:3. In embodiments, the second chemical linker includes the sequence of SEQ ID NO:4. In embodiments, the second chemical linker is the sequence of SEQ ID NO:4. In embodiments, the first receptor domain includes the sequence of SEQ ID NO:5. In embodiments, the first receptor domain is the sequence of SEQ ID NO:5. In embodiments, the recombinant cytokine receptor binding protein includes the sequence of SEQ ID NO:6. In embodiments, the recombinant cytokine receptor binding protein is the sequence of SEQ ID NO:6.

In some embodiments, the recombinant cytokine receptor binding protein comprises an amino acid sequence selected from the group consisting of SEQ ID NOs: 6, 26-28, 30, 38-48, 53-56, 60, 65-87, 99, 106, and 108-119. In some embodiments, the recombinant cytokine receptor binding protein comprises an amino acid sequence that has at least 70%, 75%, 80%, 85%, 90%, 91%, 92%, 93%, 94%, 95%, 96%, 97%, 98%, or 99% amino acid sequence identity to an amino acid sequence selected from the group consisting of SEQ ID NOs: 6, 26-28, 30, 38-48, 53-56, 60, 65-87, 99, 106, and 108-119.

In some embodiments, recombinant cytokine receptor binding protein comprises a cytokine domain comprising the amino acid sequence of SEQ ID NO: 1, an occlusion domain comprising the amino acid sequence of SEQ ID NO: 3, and a first receptor domain comprising the amino acid sequence of SEQ ID NO: 5. In some embodiments, recombinant cytokine receptor binding protein comprises a cytokine domain comprising the amino acid sequence of SEQ ID NO: 1, an occlusion domain comprising the amino acid sequence of SEQ ID NO: 51, and a first receptor domain comprising the amino acid sequence of SEQ ID NO: 5. In some embodiments, recombinant cytokine receptor binding protein comprises a cytokine domain comprising the amino acid sequence of SEQ ID NO: 1, an occlusion domain comprising the amino acid sequence of SEQ ID NO: 52, and a first receptor domain comprising the amino acid sequence of SEQ ID NO: 5.

In some embodiments, the recombinant cytokine receptor binding protein is any recombinant cytokine receptor binding protein as described in Table 2, Table 3, or Table 4.

In some embodiments, recombinant cytokine receptor binding protein comprises a cytokine domain comprising the amino acid sequence of SEQ ID NO: 1, a first chemical linker comprising an amino acid sequence selected from the group consisting of SEQ ID NOs: 2, 19, 22, 25, 34, 36, 37, 61, and 62, an occlusion domain comprising the amino acid sequence of SEQ ID NO: 3, a second chemical linker comprising an amino acid sequence selected from the group consisting of SEQ ID NOs: 9, 11, 12, 14, 15, 17, 18, 20, 21, 24, 35, and 63 and a first receptor domain comprising the amino acid sequence of SEQ ID NO: 5. In some embodiments, recombinant cytokine receptor binding protein comprises a cytokine domain comprising the amino acid sequence of SEQ ID NO: 1, a first chemical linker comprising an amino acid sequence selected from the group consisting of SEQ ID NOs: 2, 19, 22, 25, 34, 36, 37, 61, and 62, an occlusion domain comprising the amino acid sequence of SEQ ID NO: 3, a second chemical linker comprising an amino acid sequence selected from the group consisting of SEQ ID NOs: 9, 11, 12, 14, 15, 17, 18, 20, 21, 24, 35, and 63 a first receptor domain comprising the amino acid sequence of SEQ ID NO: 5, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 1, a first chemical linker comprising an amino acid sequence selected from the group consisting of SEQ ID NOs: 2, 19, 22, 25, 34, 36, 37, 61, and 62, an occlusion domain comprising the amino acid sequence of SEQ ID NO: 103, a second chemical linker comprising an amino acid sequence selected from the group consisting of SEQ ID NOs: 9, 11, 12, 14, 15, 17, 18, 20, 21, 24, 35, and 63, and a first receptor domain comprising the amino acid sequence of SEQ ID NO: 90. In some embodiments, recombinant cytokine receptor binding protein comprises a cytokine domain comprising the amino acid sequence of SEQ ID NO: 1, a first chemical linker comprising an amino acid sequence selected from the group consisting of SEQ ID NOs: 2, 19, 22, 25, 34, 36, 37, 61, and 62, an occlusion domain comprising the amino acid sequence of SEQ ID NO: 103, a second chemical linker comprising an amino acid sequence selected from the group consisting of SEQ ID NOs: 9, 11, 12, 14, 15, 17, 18, 20, 21, 24, 35, and 63, a first receptor domain comprising the amino acid sequence of SEQ ID NO: 90, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 1, a first chemical linker comprising an amino acid sequence selected from the group consisting of SEQ ID NOs: 2, 19, 22, 25, 34, 36, 37, 61, and 62, an occlusion domain comprising an amino acid sequence selected from the group consisting of SEQ ID NOs: 3, 51, 52, 88, 89, 91, 93, 95-98, 102, 103, and 107, a second chemical linker comprising an amino acid sequence selected from the group consisting of SEQ ID NOs: 9, 11, 12, 14, 15, 17, 18, 20, 21, 24, 35, and 63, and a first receptor domain comprising an amino acid sequence selected from the group consisting of SEQ ID NOs: 5, 50, 59, 90, 100, 101, and 105. In some embodiments, recombinant cytokine receptor binding protein comprises a cytokine domain comprising the amino acid sequence of SEQ ID NO: 1, a first chemical linker comprising an amino acid sequence selected from the group consisting of SEQ ID NOs: 2, 19, 22, 25, 34, 36, 37, 61, and 62, an occlusion domain comprising an amino acid sequence selected from the group consisting of SEQ ID NOs: 3, 51, 52, 88, 89, 91, 93, 95-98, 102, 103, and 107, a second chemical linker comprising an amino acid sequence selected from the group consisting of SEQ ID NOs: 9, 11, 12, 14, 15, 17, 18, 20, 21, 24, 35, and 63, a first receptor domain comprising an amino acid sequence selected from the group consisting of SEQ ID NOs: 5, 50, 59, 90, 100, 101, and 105, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 1, a first chemical linker comprising the amino acid sequence selected of SEQ ID NO: 2, an occlusion domain comprising the amino acid sequence of SEQ ID NO: 103, a second chemical linker comprising the amino acid sequence of SEQ ID NO: 15, and a first receptor domain comprising the amino acid sequence of SEQ ID NO: 90. In some embodiments, recombinant cytokine receptor binding protein comprises a cytokine domain comprising the amino acid sequence of SEQ ID NO: 1, a first chemical linker comprising the amino acid sequence selected of SEQ ID NO: 2, an occlusion domain comprising the amino acid sequence of SEQ ID NO: 103, a second chemical linker comprising the amino acid sequence of SEQ ID NO: 15, a first receptor domain comprising the amino acid sequence of SEQ ID NO: 90, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 1, a cleavage site comprising the amino acid sequence of SEQ ID NO: 29 or 64, an occlusion domain comprising an amino acid sequence selected from the group consisting of SEQ ID NOs: 3, 51, 52, 88, 89, 91, 93, 95-98, 102, 103, and 107, and a first receptor domain comprising an amino acid sequence selected from the group consisting of SEQ ID NOs: 5, 50, 59, 90, 100, 101, and 105. In some embodiments, recombinant cytokine receptor binding protein comprises a cytokine domain comprising the amino acid sequence of SEQ ID NO: 1, a cleavage site comprising the amino acid sequence of SEQ ID NO: 29 or 64, an occlusion domain comprising an amino acid sequence selected from the group consisting of SEQ ID NOs: 3, 51, 52, 88, 89, 91, 93, 95-98, 102, 103, and 107, a first receptor domain comprising an amino acid sequence selected from the group consisting of SEQ ID NOs: 5, 50, 59, 90, 100, 101, and 105, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 92, a first chemical linker comprising an amino acid sequence selected from the group consisting of SEQ ID NOs: 2, 19, 22, 25, 34, 36, 37, 61, and 62, an occlusion domain comprising an amino acid sequence selected from the group consisting of SEQ ID NOs: 3, 51, 52, 88, 89, 91, 93, 95-98, 102, 103, and 107, a second chemical linker comprising an amino acid sequence selected from the group consisting of SEQ ID NOs: 9, 11, 12, 14, 15, 17, 18, 20, 21, 24, 35, and 63, and a first receptor domain comprising the amino acid sequence of SEQ ID NO: 94. In some embodiments, recombinant cytokine receptor binding protein comprises a cytokine domain comprising the amino acid sequence of SEQ ID NO: 92, a first chemical linker comprising an amino acid sequence selected from the group consisting of SEQ ID NOs: 2, 19, 22, 25, 34, 36, 37, 61, and 62, an occlusion domain comprising an amino acid sequence selected from the group consisting of SEQ ID NOs: 3, 51, 52, 88, 89, 91, 93, 95-98, 102, 103, and 107, a second chemical linker comprising an amino acid sequence selected from the group consisting of SEQ ID NOs: 9, 11, 12, 14, 15, 17, 18, 20, 21, 24, 35, and 63, a first receptor domain comprising the amino acid sequence of SEQ ID NO: 94, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 92, a first chemical linker comprising an amino acid sequence selected from the group consisting of SEQ ID NOs: 2, 19, 22, 25, 34, 36, 37, 61, and 62, an occlusion domain comprising the amino acid sequence of SEQ ID NO: 93 or 103, a second chemical linker comprising an amino acid sequence selected from the group consisting of SEQ ID NOs: 9, 11, 12, 14, 15, 17, 18, 20, 21, 24, 35, and 63, and a first receptor domain comprising the amino acid sequence of SEQ ID NO: 94. In some embodiments, recombinant cytokine receptor binding protein comprises a cytokine domain comprising the amino acid sequence of SEQ ID NO: 92, a first chemical linker comprising an amino acid sequence selected from the group consisting of SEQ ID NOs: 2, 19, 22, 25, 34, 36, 37, 61, and 62, an occlusion domain comprising the amino acid sequence of SEQ ID NO: 93 or 103, a second chemical linker comprising an amino acid sequence selected from the group consisting of SEQ ID NOs: 9, 11, 12, 14, 15, 17, 18, 20, 21, 24, 35, and 63, a first receptor domain comprising the amino acid sequence of SEQ ID NO: 94, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 92, a cleavage site comprising the amino acid sequence of SEQ ID NO: 29 or 64, an occlusion domain comprising the amino acid sequence of SEQ ID NO: 93 or 103, and a first receptor domain comprising the amino acid sequence of SEQ ID NO: 94. In some embodiments, recombinant cytokine receptor binding protein comprises a cytokine domain comprising the amino acid sequence of SEQ ID NO: 92, a cleavage site comprising the amino acid sequence of SEQ ID NO: 29 or 64, an occlusion domain comprising the amino acid sequence of SEQ ID NO: 93 or 103, a first receptor domain comprising the amino acid sequence of SEQ ID NO: 94, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 1, a first chemical linker comprising the amino acid sequence of SEQ ID NO: 2, an occlusion domain comprising the amino acid sequence of SEQ ID NO: 3, a second chemical linker comprising the amino acid sequence of SEQ ID NO: 9, and a first receptor domain comprising the amino acid sequence of SEQ ID NO: 5. In some embodiments, recombinant cytokine receptor binding protein comprises a cytokine domain comprising the amino acid sequence of SEQ ID NO: 1, a first chemical linker comprising the amino acid sequence of SEQ ID NO: 2, an occlusion domain comprising the amino acid sequence of SEQ ID NO: 3, a second chemical linker comprising the amino acid sequence of SEQ ID NO: 9, a first receptor domain comprising the amino acid sequence of SEQ ID NO: 5, and a linker with a histidine tag comprising the amino acid sequence of SEQ ID NO: 57.

In some embodiments, recombinant cytokine receptor binding protein comprises a cytokine domain comprising the amino acid sequence of SEQ ID NO: 1, a first chemical linker comprising the amino acid sequence of SEQ ID NO: 2, an occlusion domain comprising the amino acid sequence of SEQ ID NO: 3, a second chemical linker comprising the amino acid sequence of SEQ ID NO: 15, and a first receptor domain comprising the amino acid sequence of SEQ ID NO: 5. In some embodiments, recombinant cytokine receptor binding protein comprises a cytokine domain comprising the amino acid sequence of SEQ ID NO: 1, a first chemical linker comprising the amino acid sequence of SEQ ID NO: 2, an occlusion domain comprising the amino acid sequence of SEQ ID NO: 3, a second chemical linker comprising the amino acid sequence of SEQ ID NO: 15, a first receptor domain comprising the amino acid sequence of SEQ ID NO: 5, and a linker with a histidine tag comprising the amino acid sequence of SEQ ID NO: 57.

### Nucleic Acid and Cell Compositions

In one aspect, an isolated nucleic acid is provided. The nucleic acid encodes a recombinant cytokine receptor binding protein provided herein including embodiments thereof. In embodiments, the nucleic acid includes the sequence of SEQ ID NO:7. In embodiments, the nucleic acid is the sequence of SEQ ID NO:7. In some embodiments, the nucleic acid comprises the nucleotide sequence of SEQ ID NO: 8 or SEQ ID NO: 49. In some embodiments, the nucleic acid is the nucleotide sequence of SEQ ID NO: 8 or SEQ ID NO: 49. In some embodiments, the nucleic acid comprises the nucleotide sequence of SEQ ID NO: 49. In some embodiments, the nucleic acid comprises a nucleotide sequence that has at least 75%, 80%, 85%, 90%, 91%, 92%, 93%, 94%, 95%, 96%, 97%, 98%, or 99% nucleotide sequence identity to the nucleotide sequence of SEQ ID NO: 7, SEQ ID NO: 8, or SEQ ID NO: 49 across the whole sequence or a portion of the sequence. In some embodiments, the nucleic acid comprises a nucleotide sequence encoding an amino acid sequence selected from the group consisting of SEQ ID NOs: 6, 26-28, 30, 38-48, 53-56, 60, 65-87, 99, 106, and 108-119.

In another aspect, an expression vector including the nucleic acid provided herein including embodiments thereof is provided. In embodiments, the expression vector is a viral vector. In embodiments, the virus is a lentivirus or onco-retrovirus. In embodiments, the virus is a lentivirus. In embodiments, the virus is an onco-retrovirus.

In another aspect, a T lymphocyte including the expression vector provided herein including embodiments thereof is provided. In embodiments, the T cell further includes a chimeric antigen receptor.

### Pharmaceutical Compositions

Pharmaceutical compositions provided by the present invention (e.g., proteins provided herein) include compositions wherein the active ingredient (e.g. compositions described herein, including embodiments or examples) is contained in a therapeutically effective amount, i.e., in an amount effective to achieve its intended purpose. The pharmaceutical compositions provided herein include compositions comprising any of the recombinant cytokine receptor binding proteins described herein. In some embodiments, the pharmaceutical composition comprises any of the recombinant cytokine receptor binding proteins described herein and a pharmaceutically acceptable excipient. The actual amount of active ingredient effective for a particular application will depend, inter alia, on the condition being treated. When administered in methods to treat a disease, the recombinant proteins described herein will contain an amount of active ingredient effective to achieve the desired result, e.g., modulating the activity of a target molecule, and/or reducing, eliminating, or slowing the progression of disease symptoms. Determination of a therapeutically effective amount of a compound of the invention (i.e., recombinant proteins) is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods and compounds (i.e., recombinant proteins) of the invention. Adjustment and manipulation of established dosages (e.g., frequency and duration) are well within the ability of those skilled in the art.

For any composition (e.g., recombinant protein, nucleic acid) provided herein, the therapeutically effective amount can be initially determined from cell culture assays. Target concentrations will be those concentrations of active compound(s) that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound (i.e., recombinant proteins) being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound (i.e., recombinant proteins). Thereafter, the dosage is increased by small increments until the optimum effect under circumstances is reached.

Dosage amounts and intervals can be adjusted individually to provide levels of the administered compound (i.e., recombinant proteins) effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds (i.e., recombinant proteins) of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

In an aspect is provided a pharmaceutical composition including a recombinant cytokine receptor binding protein as described herein, including embodiments thereof, and pharmaceutically acceptable excipient.

### Methods of Treatment

The compositions provided herein, including embodiments thereof, are contemplated as providing effective treatments for diseases such as cancer. Thus, in an aspect is provided a method including administering to a subject in need thereof a therapeutically effective amount of a recombinant cytokine receptor binding protein as described herein, including embodiments thereof.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). For example certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds (e.g., recombinant proteins) of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The compositions described herein can be used in combination with one another, with other active agents known to be useful in treating a cancer such as anti-cancer agents.

Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. In embodiments, the compositions herein may be used in combination with adjunctive agents that may not be effective alone, but may contribute to the efficacy of the active agent in treating cancer.

In embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In embodiments, co-administration can be accomplished by co-formulation, i.e., preparing a single pharmaceutical composition including both active agents. In embodiments, the active agents can be formulated separately. In embodiments, the active and/or adjunctive agents may be linked or conjugated to one another.

### Definitions

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6 kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain. Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805, which are incorporated, by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

A “single domain antibody” as provided herein refers to an antibody fragment including a single monomeric variable antibody domain. Like a whole antibody, a single domain antibody is able to bind selectively to a specific antigen. The molecular weight of a single domain antibody is 12-15 kDa, single domain antibody. In embodiments, a single domain antibody is a variable heavy chain domain. In embodiments, a single domain antibody is a variable light chain domain. Non-limiting examples of single domain antibodies include camelid-derived VHH fragments and VNAR (variable immunoglobulin new antigen receptor) fragments. In embodiments, the single-domain antibody is a peptide domain of about 110 amino acids. In embodiments, the single-domain antibody includes a variable heavy chain domain. In embodiments, the single-domain antibody includes a variable light chain domain.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected protein or protein domain (e.g., cytokine or occlusion domain) corresponds to leucine at position 40 of IL-2 or a fragment thereof, when the selected residue occupies the same essential spatial or other structural relationship as a leucine at position 40 of IL-2. In some embodiments, where a selected protein or protein domain is aligned for maximum homology with, for example, IL-2 or a fragment thereof, the position in the aligned selected protein aligning with leucine 40 is said to correspond to threonine 40. Instead of a primary sequence alignment, a three dimensional structural alignment can also be used, e.g., where the structure of the selected protein is aligned for maximum correspondence with the leucine at position 40 of for example, IL-2 or a fragment thereof, and the overall structures compared. In this case, an amino acid that occupies the same essential position as leucine 40 in the structural model, is said to correspond to the leucine 40 residue.

Likewise, a selected residue in a selected protein or protein domain (e.g., a cytokine domain, a first receptor domain or an occlusion domain) corresponds to a residue at position 45, when the selected residue occupies the same essential spatial or other structural position within the protein or protein domain as the residue at position 45. In some embodiments, where a selected protein or protein domain is aligned for maximum homology with, the position in the aligned selected protein or protein domain (e.g., a second ligand binding domain or a second ligand binding domain enhancer) aligning with position 45 is said to correspond to position 45. Instead of a primary sequence alignment, a three dimensional structural alignment can also be used, e.g., where the structure of the selected protein or protein domain is aligned for maximum correspondence with the residue at position 45, and the overall structures compared. In this case, an amino acid that occupies the same essential position as residue 45 in the structural model is said to correspond to the 45 residue.

The term “interleukin 2” or “IL-2” as referred to herein includes any of the recombinant or naturally-occurring forms of interleukin (IL)-2 protein or variants or homologs thereof that maintain IL-2 activity (e.g. within at least 50%, 80%, 90%, 95%, 96%, 97%, 98%, 99% or 100% activity compared to IL-2 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 protein. In embodiments, the IL-2 protein is substantially identical to the protein identified by the UniProt reference number: P60568 or a variant or homolog having substantial identity thereto.

The term “IL-2RA” or “IL-2 receptor α” as referred to herein includes any of the recombinant or naturally-occurring forms of interleukin (IL)-2 receptor alpha protein or variants or homologs thereof that maintain IL-2 receptor alpha activity (e.g. within at least 50%, 80%, 90%, 95%, 96%, 97%, 98%, 99% or 100% activity compared to IL-2 receptor alpha protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 receptor alpha protein. In embodiments, the IL-2 receptor alpha protein is substantially identical to the protein identified by the UniProt reference number: P01589.

The term “IL-2RB” or “IL-2 receptor (3” as referred to herein includes any of the recombinant or naturally-occurring forms of interleukin (IL)-2 receptor beta protein or variants or homologs thereof that maintain IL-2 receptor beta activity (e.g. within at least 50%, 80%, 90%, 95%, 96%, 97%, 98%, 99% or 100% activity compared to IL-2 receptor beta protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 receptor beta protein. In embodiments, the IL-2 receptor beta protein is substantially identical to the protein identified by the UniProt reference number: P14784.

The term “IL-2RG” or “IL-2 receptor γ” as referred to herein includes any of the recombinant or naturally-occurring forms of interleukin (IL)-2 receptor gamma protein or variants or homologs thereof that maintain IL-2 receptor gamma activity (e.g. within at least 50%, 80%, 90%, 95%, 96%, 97%, 98%, 99% or 100% activity compared to IL-2 receptor gamma protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 receptor gamma protein. In embodiments, the IL-2 receptor gamma protein is substantially identical to the protein identified by the UniProt reference number: P31785.

The term “interleukin 15” or “IL-15” as referred to herein includes any of the recombinant or naturally-occurring forms of interleukin (IL)-15 protein or variants or homologs thereof that maintain IL-15 activity (e.g. within at least 50%, 80%, 90%, 95%, 96%, 97%, 98%, 99% or 100% activity compared to IL-15 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein. In embodiments, the IL-15 protein is substantially identical to the protein identified by the UniProt reference P40933 or a variant or homolog having substantial identity thereto.

The term “IL-15RA” or “IL-15 receptor α” as referred to herein includes any of the recombinant or naturally-occurring forms of IL-15 receptor alpha protein or variants or homologs thereof that maintain IL-15 receptor alpha activity (e.g. within at least 50%, 80%, 90%, 95%, 96%, 97%, 98%, 99% or 100% activity compared to IL-15 receptor alpha protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 receptor alpha protein. In embodiments, the IL-15 receptor alpha protein is substantially identical to the protein identified by the UniProt reference: Q13261 or by the NCBI Reference Sequence: NP_001230468.1.

A “ligand” refers to an agent, e.g., a polypeptide or other molecule (e.g., antigen, cytokine), capable of binding to a ligand binding domain (e.g., receptor or antibody, antibody variant, antibody region or fragment thereof).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents, which can be produced in the reaction mixture.

The term “contacting” may include allowing two species to react, interact, or physically touch (e.g., bind), wherein the two species may be, for example, a recombinant protein as described herein and a cytokine receptor. In embodiments, contacting includes, for example, allowing a recombinant protein to bind to a cancer protein expressed on a cancer cell.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “plasmid,” “expression vector,” or “viral vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids. Suitable viral vectors contemplated herein include, for example, lentiviral vectors and onco-retroviral vectors.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer) means that the disease is caused by (in whole or in part), or a symptom of the disease is caused by (in whole or in part) the substance or substance activity or function.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds (e.g., recombinant proteins) can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

A “domain” as provided herein refers to a structural portion of a protein or fragment thereof. A domain as provided herein may exist independently of the rest of the protein or fragment thereof. In embodiments, a domain forms a three-dimensional structure and is independently stable. A domain may have the same functional properties as the protein it is derived from.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “cytokine” as used herein refers to a protein or fragment thereof involved in cell signaling. A cytokine may have a size from about 5 to about 20 kDa and may be involved in autocrine signaling, paracrine signaling or endocrine signaling. Cytokines include, without limitation, chemokines, interferons, interleukins, lymphokines, and tumor necrosis factors and may be produced by a broad range of cells, including immune cells (e.g., macrophages, B lymphocytes, T lymphocytes and mast cells), endothelial cells, fibroblasts, and stromal cells. Cytokines may be produced by more than one type of cell. By binding to their cognate receptors, cytokines may modulate the balance between humoral and cell-based immune responses, and regulate the maturation, growth, and responsiveness of particular cell populations.

The term “Fc binding peptide” as used herein refers to a peptide that is capable of binding to serum IgG, such as a peptide that includes the amino acid sequence DCAWHLGELVWCT (SEQ ID NO: 31). See, e.g., DeLano et al., Science (2000); 287(5456): 1279-83. By fusing an Fc binding peptide to a recombinant protein, such as any of the recombinant cytokine receptor binding proteins described herein, the half-life of the fusion protein can be increased by enabling the fusion protein to bind to serum IgG. The component of the recombinant cytokine receptor binding protein that the Fc binding peptide can be linked to is not limited. Accordingly, the Fc binding peptide can, in some embodiments, be linked to a first chemical linker, a second chemical linker, an occlusion domain, a cytokine domain, or a first receptor domain. The Fc binding domain can, in some embodiments, be included in a first chemical linker or a second chemical linker.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Exemplary Embodiments

Among the provided embodiments are:

1. A recombinant cytokine receptor binding protein comprising:

a cytokine domain comprising a first receptor binding site and a second receptor binding site;

a first receptor domain that specifically binds to said first receptor binding site; and

an occlusion domain positioned to sterically hinder binding of said second receptor binding site to a cognate receptor of said second receptor binding site; wherein
the C-terminus of said cytokine domain is bound to the N-terminus of said occlusion domain through a first chemical linker; and
the C-terminus of said occlusion domain is bound to the N-terminus of said first receptor domain through a second chemical linker.
   2. A recombinant cytokine receptor binding protein comprising:

a cytokine domain comprising a first receptor binding site and a second receptor binding site;

a first receptor domain that specifically binds to said first receptor binding site; and

an occlusion domain positioned to sterically hinder binding of said second receptor binding site to a cognate receptor of said second receptor binding site; wherein
the C-terminus of said occlusion domain is bound to the N-terminus of said cytokine domain through a first chemical linker; and
   the C-terminus of said first receptor domain is bound to the N-terminus of said occlusion domain through a second chemical linker.
   3. The recombinant cytokine receptor binding protein of embodiment 1 or embodiment 2, wherein said cytokine domain comprises a third receptor binding site and wherein said occlusion domain is positioned to sterically hinder binding of said third receptor binding site to said cognate receptor.
   4. The recombinant cytokine receptor binding protein of any one of embodiments 1-3, wherein said cytokine domain is an IL-2 domain.
   5. The recombinant cytokine receptor binding protein of embodiment 4, wherein said IL-2 domain comprises the amino acid sequence of SEQ ID NO: 1.
   6. The recombinant cytokine receptor binding protein of embodiment 4, wherein said first receptor binding site is an IL-2Rα binding site.
   7. The recombinant cytokine receptor binding protein of embodiment 6, wherein said second receptor binding site is an IL-2Rγ binding site.
   8. The recombinant cytokine receptor binding protein of embodiment 6, wherein said second receptor binding site is an IL-2Rβ binding site.
   9. The recombinant cytokine receptor binding protein of embodiment 3, wherein said third receptor binding site is an IL-2Rγ binding site.
   10. The recombinant cytokine receptor binding protein of embodiment 3, wherein said third receptor binding site is an IL-2Rβ binding site.
   11. The recombinant cytokine receptor binding protein of any one of embodiments 1-10, wherein said first receptor domain binds non-covalently to said first receptor binding site.
   12. The recombinant cytokine receptor binding protein of any one of embodiments 1-11, wherein said first receptor domain comprises an amino acid sequence selected from the group consisting of SEQ ID NOs: 5, 50, 59, 90, 100, 101, and 105.
   13. The recombinant cytokine receptor binding protein of any one of embodiments 1-3, wherein said cytokine domain is an IL-15 domain.
   14. The recombinant cytokine receptor binding protein of embodiment 13, wherein said IL-15 domain comprises the amino acid sequence of SEQ ID NO: 92.
   15. The recombinant cytokine receptor binding protein of any one of embodiments 1-3, 13 and 14, wherein said first receptor domain binds non-covalently to said first receptor binding site.
   16. The recombinant cytokine receptor binding protein of any one of embodiments 1-3 and 13-15, wherein said first receptor domain comprises the amino acid sequence of SEQ ID NO: 94.
   17. The recombinant cytokine receptor binding protein of any one of embodiments 1-16, wherein said occlusion domain comprises an amino acid sequence selected from the group consisting of SEQ ID NOs: 3, 51, 52, 88, 89, 91, 93, 95-98, 102, 103, and 107.
   18. The recombinant cytokine receptor binding protein of any one of embodiments 1-17, wherein said occlusion domain is a protein domain.
   19. The recombinant cytokine receptor binding protein of any one of embodiments 1-18, wherein said occlusion domain is an antibody domain.
   20. The recombinant cytokine receptor binding protein of embodiment 19, wherein said antibody domain is an Fc domain.
   21. The recombinant cytokine receptor binding protein of embodiment 20, wherein said Fc domain comprises a constant heavy chain 2 (CH2) domain or a constant heavy chain 3 (CH3) domain.
   22. The recombinant cytokine receptor binding protein of embodiment 20, wherein said Fc domain is a CH2 domain.
   23. The recombinant cytokine receptor binding protein of embodiment 20, wherein said Fc domain comprises a CH2 domain and a CH3 domain.
   24. The recombinant cytokine receptor binding protein of embodiment 20, wherein said Fc domain is a CH2 domain covalently linked to a CH3 domain.
   25. The recombinant cytokine receptor binding protein of any one of embodiments 21-24, wherein said CH2 domain is an IgG1 CH2 domain.
   26. The recombinant cytokine receptor binding protein of any one of embodiments 20-25, wherein said Fc domain comprises an amino acid sequence selected from the group consisting of SEQ ID NOs: 3, 88, 89, 91, 93, 103, and 107.
   27. The recombinant cytokine receptor binding protein of any one of embodiments 1-19, wherein said occlusion domain is a Fab domain.
   28. The recombinant cytokine receptor binding protein of embodiment 27, wherein said Fab domain comprises the amino acid sequence of SEQ ID NO: 102.
   29. The recombinant cytokine receptor binding protein of embodiment 27, wherein said Fab domain comprises a variable light chain domain and a constant light chain domain.
   30. The recombinant cytokine receptor binding protein of embodiment 27, wherein said Fab domain is a variable light chain domain bound to a constant light chain domain.
   31. The recombinant cytokine receptor binding protein of any one of embodiments 27-30, wherein said Fab domain comprises the amino acid sequence of SEQ ID NO: 51.
   32. The recombinant cytokine receptor binding protein of embodiment 27, wherein said Fab domain comprises a variable heavy chain domain and a constant heavy chain domain.
   33. The recombinant cytokine receptor binding protein of embodiment 27, wherein said Fab domain is a variable heavy chain domain bound to a constant heavy chain domain.
   34. The recombinant cytokine receptor binding protein of embodiment 32 or embodiment 33, wherein said Fab domain comprises the amino acid sequence of SEQ ID NO: 52.
   35. The recombinant cytokine receptor binding protein of any one of embodiments 1-19, wherein said occlusion domain is a nanobody domain.
   36. The recombinant cytokine receptor binding protein of embodiment 35, wherein said nanobody domain comprises the amino acid sequence of SEQ ID NO: 95 or 97.
   37. The recombinant cytokine receptor binding protein of embodiment 35, wherein said nanobody domain is a variable heavy chain domain.
   38. The recombinant cytokine receptor binding protein of embodiment 35, wherein said nanobody domain is a variable light chain domain.
   39. The recombinant cytokine receptor binding protein of any one of embodiments 1-19, wherein said occlusion domain is a protein L domain.
   40. The recombinant cytokine receptor binding protein of embodiment 39, wherein said protein L domain comprises the amino acid sequence of SEQ ID NO: 96.
   41. The recombinant cytokine receptor binding protein of any one of embodiments 1-19, wherein said occlusion domain is a CD16 domain.
   42. The recombinant cytokine receptor binding protein of embodiment 41, wherein said CD16 domain comprises the amino acid sequence of SEQ ID NO: 98.
   43. The recombinant cytokine receptor binding protein of any one of embodiments 1-42, wherein said cognate receptor comprises an IL-2 receptor β or fragment thereof or an IL-2 receptor γ or fragment thereof.
   44. The recombinant cytokine receptor binding protein of any one of embodiments 1-43, wherein said first chemical linker and said second chemical linker are independently a peptidyl linker.
   45. The recombinant cytokine receptor binding protein of embodiment 44, wherein said first chemical linker comprises an amino acid sequence selected from the group consisting of SEQ ID NOs: 2, 4, 9-25, 29, 34-37, 61-64, and 104.
   46. The recombinant cytokine receptor binding protein of embodiment 44 or embodiment 45, wherein said second chemical linker comprises an amino acid sequence selected from the group consisting of SEQ ID NOs: 2, 4, 9-25, 29-34-37, 61-64, and 104.
   47. The recombinant cytokine receptor binding protein of any one of embodiments 1-46, wherein said first chemical linker and said second chemical linker are independently a cleavable linker.
   48. The recombinant cytokine receptor binding protein of any one of embodiments 1-46, wherein said first chemical linker or said second chemical linker is a cleavable linker.
   49. The recombinant cytokine receptor binding protein of embodiment 47 or embodiment 48, wherein said cleavable linker is a protease cleavable linker.
   50. The recombinant cytokine receptor binding protein of any one of embodiments 47-49, wherein said cleavable linker is a tumor-associated protease cleavable linker.
   51. The recombinant cytokine receptor binding protein of any one of embodiments 47-50, wherein said cleavable linker comprises the amino acid sequence of SEQ ID NO: 29 or 64.
   52. The recombinant cytokine receptor binding protein of any one of embodiments 47-51, wherein said cleavable linker comprises an amino acid sequence selected from the group consisting of SEQ ID NOs: 2, 23-25, 34, 36, 37, 61, and 62.
   53. The recombinant cytokine receptor binding protein of any one of embodiments 1-52, wherein said first chemical linker and said second chemical linker independently have a length of less than about 30 amino acids.
   54. The recombinant cytokine receptor binding protein of any one of embodiments 1-53, wherein said first chemical linker and said second chemical linker independently have a length of about 3-8 amino acids.
   55. The recombinant cytokine receptor binding protein of any one of embodiments 1-54, wherein said first chemical linker and said second chemical linker independently have a length of 5 or 6 amino acids.
   56. The recombinant cytokine receptor binding protein of any one of embodiments 1-55, wherein said recombinant cytokine receptor binding protein comprises an Fc binding peptide.
   57. The recombinant cytokine receptor binding protein of embodiment 56, wherein said Fc binding peptide comprises an amino acid sequence selected from the group consisting of SEQ ID NOs: 31-33.
   58. The recombinant cytokine receptor binding protein of embodiment 1 or embodiment 2, wherein said recombinant cytokine receptor binding protein comprises an amino acid sequence selected from the group consisting of SEQ ID NOs: 6, 26-28, 30, 38-48, 53-56, 60, 65-87, 99, 106, and 108-119.
   59. A recombinant cytokine receptor binding protein comprising:

an IL-2 domain comprising an IL-2 receptor α binding site, an IL-2 receptor β binding site and an IL-2 receptor γ binding site;

an IL-2 receptor α domain that specifically binds to said IL-2 receptor α binding site; and

an Fc domain positioned to sterically hinder binding of said IL-2 receptor β binding site to an IL-2 receptor β domain; wherein the C-terminus of said IL-2 domain is bound to the N-terminus of said Fc domain through a cleavable linker; and
the C-terminus of said Fc domain is bound to the N-terminus of said IL2 receptor α domain through a chemical linker.
   60. The recombinant cytokine receptor binding protein of embodiment 59, wherein said Fc domain is positioned to sterically hinder binding of said IL-2 receptor γ binding site to an IL-2 receptor γ domain.
   61. The recombinant cytokine receptor binding protein of embodiment 59 or embodiment 60, wherein said IL-2 domain comprises the amino acid sequence of SEQ ID NO: 1.
   62. The recombinant cytokine receptor binding protein of any one of embodiments 59-61, wherein said IL-2 receptor α domain comprises an amino acid sequence selected from the group consisting of SEQ ID NOs: 5, 50, 59, 90, 100, 101, and 105.
   63. The recombinant cytokine receptor binding protein of any one of embodiments 59-62, wherein said Fc domain comprises an amino acid sequence selected from the group consisting of SEQ ID NOs: 3, 88, 89, 91, 93, 103, and 107.
   64. The recombinant cytokine receptor binding protein of any one of embodiments 59-63, wherein said cleavable linker comprises an amino acid sequence selected from the group consisting of SEQ ID NOs: 2, 23-25, 29-34, 36, 37, 61, 62, and 64.
   65. The recombinant cytokine receptor binding protein of any one of embodiments 59-64, wherein said chemical linker comprises an amino acid sequence selected from the group consisting of SEQ ID NOs: 4, 9, 11, 12, 14, 15, 17, 18, 20, 21, 24, 35, and 63.
   66. The recombinant cytokine receptor binding protein of any one of embodiments 59-65, wherein said recombinant cytokine receptor binding protein comprises an Fc binding peptide.
   67. The recombinant cytokine receptor binding protein of embodiment 66, wherein said Fc binding peptide comprises an amino acid sequence selected from the group consisting of SEQ ID NOs: 31-33.
   68. A pharmaceutical composition comprising a recombinant cytokine receptor binding protein of any one of embodiments 1-67 and a pharmaceutically acceptable excipient.
   69. An isolated nucleic acid encoding a recombinant cytokine receptor binding protein of any one of embodiments 1-67.
   70. The isolated nucleic acid of embodiment 69, wherein the isolated nucleic acid comprises a nucleotide sequence selected from the group consisting of SEQ ID NOs: 7, 8, and 49.
   71. An expression vector comprising the nucleic acid of embodiment 69 or embodiment 70.
   72. The expression vector of embodiment 71, wherein said expression vector is a viral vector.
   73. The expression vector of embodiment 72, wherein said viral vector is a lentivirus or onco-retrovirus.
   74. A T lymphocyte comprising the expression vector of any one of embodiments 71-73.
   75. The T lymphocyte of embodiment 74, wherein said T cell further comprises a chimeric antigen receptor.
   76. A method of treating cancer in subject in need thereof, said method comprising administering to a subject a therapeutically effective amount of a recombinant cytokine receptor binding protein of any one of embodiments 1-67.

### EXAMPLES

Examples are provided below to facilitate a more complete understanding of the invention. However, the scope of the invention is not limited to specific embodiments disclosed in these Examples, which are for purposes of illustration only.

### Example 1: Construction of Exemplary Cytokine Receptor Binding Proteins

In some exemplary constructs of the cytokine receptor binding protein, the CH2 domain of the human IgG1, as an exemplary occlusion domain, is nested between IL2 and IL-2Rα. Other designs, including the use of a Fab domain, nanobody, protein L, CD16, or other biologic, as exemplary occlusion domains, are also constructed. The approach is also applied for IL15 and other cytokines.

In this format, a number of constructs are produced, varying the length of linker between the CH2 domain and the IL2 and IL2rα domains. In addition, different orders of the molecule are constructed (e.g., IL2rα-CH2-IL2). Optionally, the CH2 domain is switched for a monovalent Fc construct.

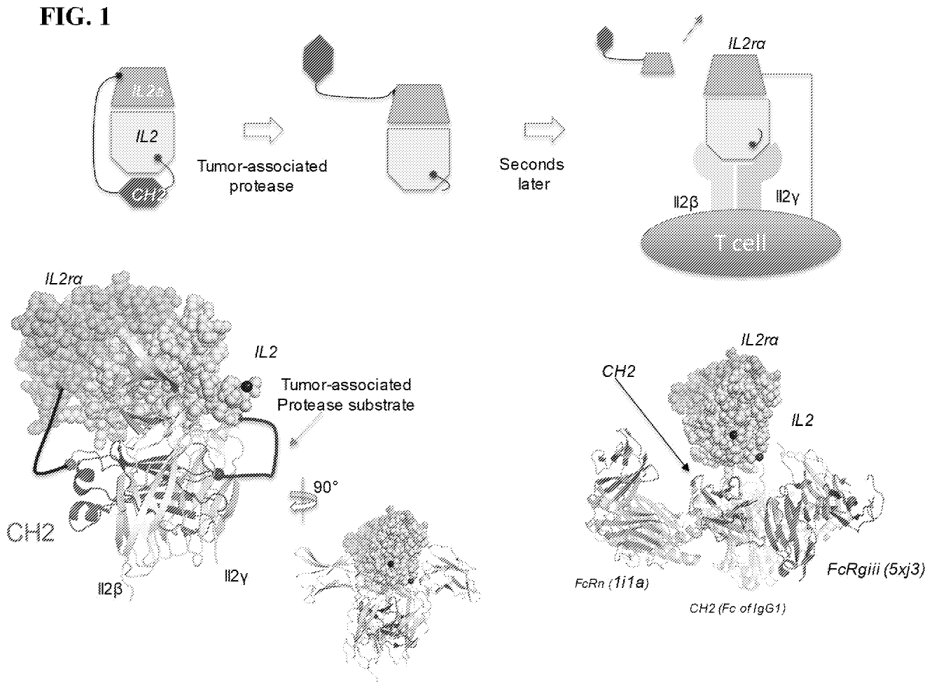

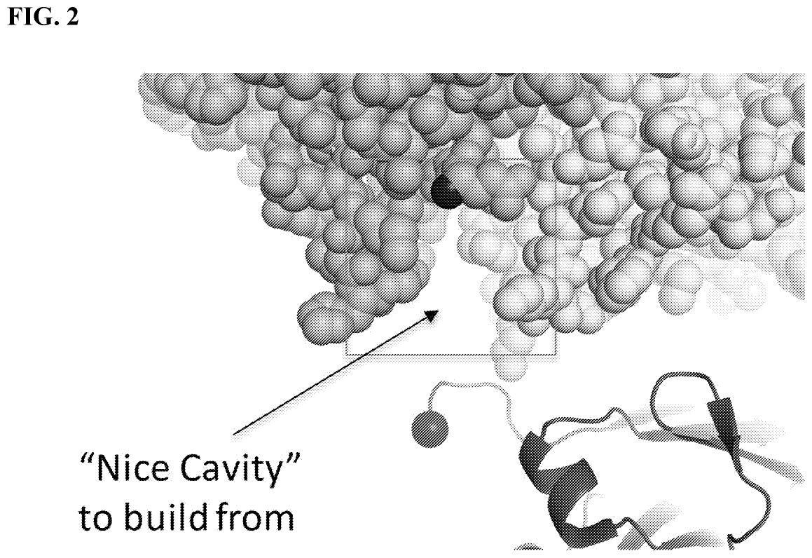

An illustration of a proposed mechanism of action is provided in FIG. 1. The lines represent linkers. Constructs are designed to alter or improve the PK and/or biodistribution.

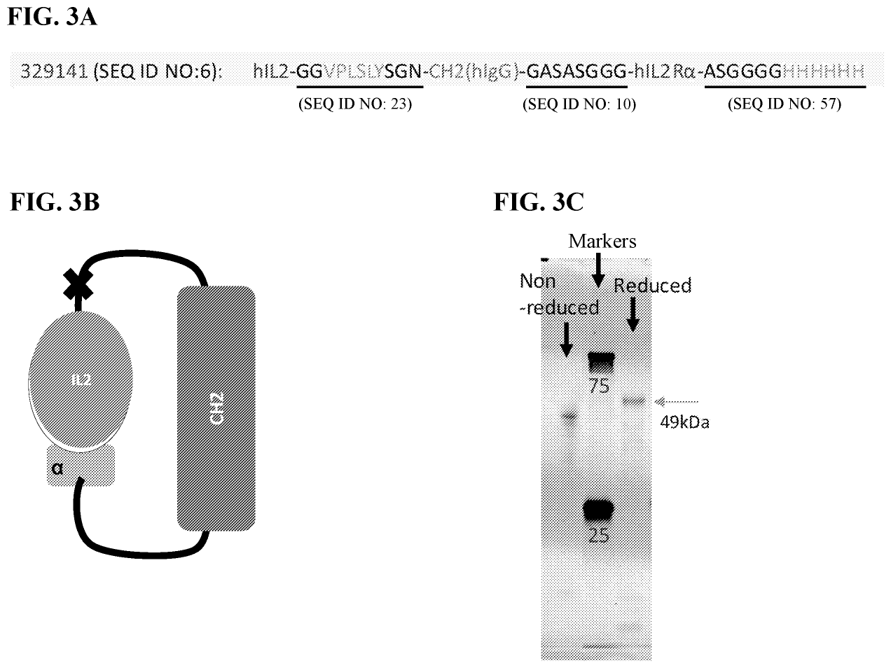

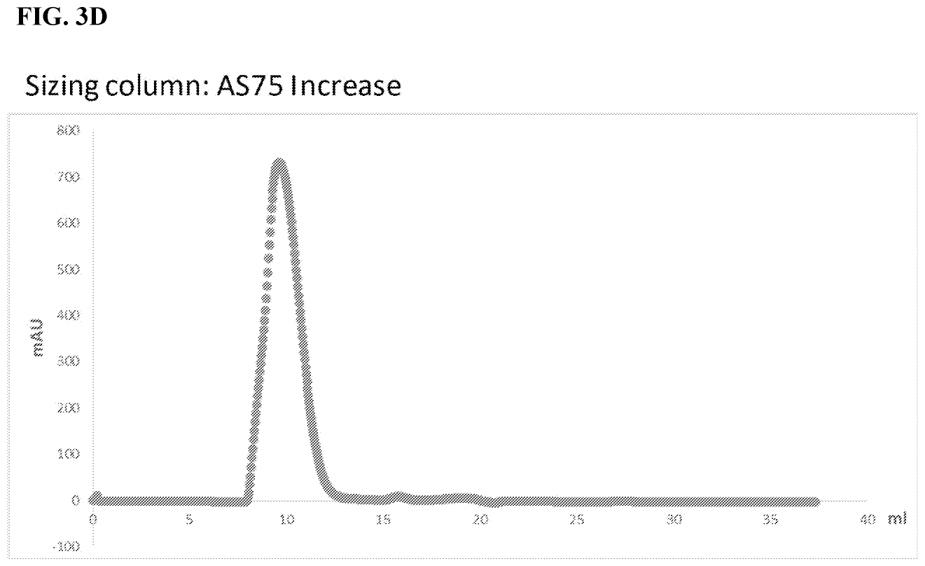

A design of an exemplary cytokine receptor binding protein referred to as 329141 is depicted in FIGS. 3A and 3B. An SDS-PAGE analysis of the 329141 construct in non-reduced (left lane) and reduced (right lane) conditions is depicted in FIG. 3C. The expected mass of the 329141 construct is 49,625 D. An analysis of the 329141 construct using size exclusion chromatography (SEC) is depicted in FIG. 3C.

Variants of the exemplary 329141 construct were created by modifying the linker between the CH2 domain and the IL-2Rα. These variants include, for instance, the constructs termed 329141A, 329141B, 329141C, and 329141D, with modifications to the second chemical linker reflected in the sequences of the second chemical linker, as shown in Table 1.

TABLE 1

| | Sequence of the second chemical |
|---|---|
| | linker (linker between the CH2 |
| Construct ID | domain and the IL-2Rα) |
| 329141 | KGQPGASASGGGMLSL (SEQ ID NO: 9) |
| 329141A | GGASASAGG (SEQ ID NO: 11) |
| 329141B | KGQPGASASGMLSL (SEQ ID NO: 12) |
| 329141C | KGGASASGMLSL (SEQ ID NO: 14) |
| 329141D | SGGASASGSLSL (SEQ ID NO: 15) |

Additional exemplary cytokine receptor binding protein constructs were designed. In some constructs, an Fc binding peptide (e.g., having the amino acid sequence of SEQ ID NO: 31, SEQ ID NO: 32, or SEQ ID NO: 33) is incorporated into a first chemical linker or a second chemical linker in the cytokine receptor binding protein. Exemplary constructs that include an Fc binding peptide include, for instance, the 1022, 1023, 1024, 1029, 1030, and 1031 constructs. By incorporating an Fc binding peptide into a linker, the Fc binding peptide can be linked to the N-terminus or C-terminus of the CH2 domain (or another exemplary occlusion domain), or to the N-terminus of IL2 (or another exemplary cytokine domain, such as IL15). In some constructs, other modifications are made, such as a truncation to the CH2 domain, a monomeric CH2-CH3 domain as the Fc domain, or modifications made to one or both linkers, including a modification to render the cleavable linker non-cleavable. Some constructs were generated by further modifying the 329141D construct. Some exemplary constructs include IL2 as the cytokine domain and IL-2Rα as the first receptor domain. Some constructs include IL15 as the cytokine domain and IL-15Rα as the first receptor domain. Structural features of exemplary embodiments of cytokine receptor binding protein constructs, including exemplary sequences comprised within each embodiment, are shown below in Table 2. In Tables 2 and 3 below, the first chemical linker is also referred to as “L1”, the second chemical linker is also referred to as “L2”, a linker with a histidine-tag is also referred to as “LH”, a light chain or portion thereof is also referred to as “LC”, and a heavy chain or portion thereof is also referred to as “HC.” A third chemical linker, in some embodiments, is also referred to as “L3.”

TABLE 2

| | | | | | First | | | |
|---|---|---|---|---|---|---|---|---|
| | | | Occlusion | | receptor | Linker | | |
| | Cytokine | First | domain | Second | domain | with | | |
| | domain | chemical | (CH2, | chemical | (IL-2Rα | His- | Orientation | |
| Construct | (IL2 or | linker | LC, or | linker | or IL- | tag | (N- to C- | Exemplary |
| ID | IL15) | (L1) | HC) | (L2) | 15Rα) | (LH) | terminus) | Sequence |
| 329141 | SEQ ID | SEQ ID | SEQ ID | SEQ ID | SEQ ID | SEQ | IL2-L1- | SEQ ID |
| | NO: 1 | NO: 2 | NO: 103 | NO: 9 | NO: 90 | ID | CH2-L2- | NO: 38 |
| | (IL2) | | (CH2) | | (IL-2Rα) | NO: 57 | IL-2Rα-LH | |
| 329141A | SEQ ID | SEQ ID | SEQ ID | SEQ ID | SEQ ID | SEQ | IL2-L1- | SEQ ID |
| | NO: 1 | NO: 2 | NO: 103 | NO: 11 | NO: 90 | ID | CH2-L2- | NO: 39 |
| | (IL2) | | (CH2) | | (IL-2Rα) | NO: 57 | IL-2Rα-LH | |
| 329141B | SEQ ID | SEQ ID | SEQ ID | SEQ ID | SEQ ID | SEQ | IL2-L1- | SEQ ID |
| | NO: 1 | NO: 2 | NO: 103 | NO: 12 | NO: 90 | ID | CH2-L2- | NO: 40 |
| | (IL2) | | (CH2) | | (IL-2Rα) | NO: 57 | IL-2Rα-LH | |
| 329141C | SEQ ID | SEQ ID | SEQ ID | SEQ ID | SEQ ID | SEQ | IL2-L1- | SEQ ID |
| | NO: 1 | NO: 2 | NO: 103 | NO: 14 | NO: 90 | ID | CH2-L2- | NO: 41 |
| | (IL2) | | (CH2) | | (IL-2Rα) | NO: 57 | IL-2Rα-LH | |
| 329141D | SEQ ID | SEQ ID | SEQ ID | SEQ ID | SEQ ID | SEQ | IL2-L1- | SEQ ID |
| | NO: 1 | NO: 2 | NO: 103 | NO: 15 | NO: 90 | ID | CH2-L2- | NO: 42 |
| | (IL2) | | (CH2) | | (IL-2Rα) | NO: 57 | IL-2Rα-LH | |
| 1014 | SEQ ID | SEQ ID | SEQ ID | SEQ ID | SEQ ID | SEQ | IL2-L1-LC- | SEQ ID |
| | NO: 1 | NO: 2 | NO: 51 | NO: 20 | NO: 5 | ID | L2-IL2- | NO: 65 |
| | (IL2) | | (LC) | | (IL-2Rα) | NO: 57 | 2Rα-LH | |
| 1015 | SEQ ID | SEQ ID | SEQ ID | SEQ ID | SEQ ID | SEQ | IL2-L1-HC- | SEQ ID |
| | NO: 1 | NO: 25 | NO: 52 | NO: 21 | NO: 5 | ID | L2-IL2- | NO: 66 |
| | (IL2) | | (HC) | | (IL-2Rα) | NO: 57 | 2Rα-LH | |
| 1016 | SEQ ID | SEQ ID | SEQ ID | SEQ ID | SEQ ID | | IL-2Rα-L1- | SEQ ID |
| | NO: 1 | NO: 19 | NO: 51 | NO: 24 | NO: 100 | | LC-L2-IL2 | NO: 67 |
| | (IL2) | | (LC) | | (IL-2Rα) | | | |
| 1017 | SEQ ID | SEQ ID | SEQ ID | SEQ ID | SEQ ID | | IL-2Rα-L1- | SEQ ID |
| | NO: 1 | NO: 19 | NO: 52 | NO: 24 | NO: 100 | | HC-L2-IL2 | NO: 68 |
| | (IL2) | | (HC) | | (IL-2Rα) | | | |
| 1018 | SEQ ID | SEQ ID | SEQ ID | SEQ ID | SEQ ID | SEQ | IL2-L1-HC- | SEQ ID |
| | NO: 1 | NO: 62 | NO: 52 | NO: 63 | NO: 101 | ID | L2-IL2- | NO: 69 |
| | (IL2) | | (HC) | | (IL-2Rα) | NO: 57 | 2Rα-LH | |
| 1019 | SEQ ID | SEQ ID | SEQ ID | SEQ ID | SEQ ID | SEQ | IL2-L1-HC- | SEQ ID |
| | NO: 1 | NO: 25 | NO: 102 | NO: 21 | NO: 5 | ID | L2-IL2- | NO: 70 |
| | (IL2) | | (HC) | | (IL-2Rα) | NO: 57 | 2Rα-LH | |
| 1020 | SEQ ID | SEQ ID | SEQ ID | SEQ ID | SEQ ID | SEQ | IL15-L1- | SEQ ID |
| | NO: 92 | NO: 2 | NO: 103 | NO: 15 | NO: 94 | ID | CH2-L2- | NO: 71 |
| | (IL15) | | | | (IL- | NO: 57 | IL-15Rα- | |
| | | | | | 15Rα) | | LH | |
| 1021 | SEQ ID | SEQ ID | SEQ ID | SEQ ID | SEQ ID | SEQ | IL2-L1- | SEQ ID |
| | NO: 1 | NO: 2 | NO: 88 | NO: 15 | NO: 90 | ID | CH2-L2- | NO: 72 |
| | (IL2) | | (Truncated | | (IL-2Rα) | NO: 57 | IL-2Rα-LH | |
| | | | CH2) | | | | | |
| 1022 | SEQ ID | SEQ ID | SEQ ID | SEQ ID | SEQ ID | SEQ | IL2-L1- | SEQ ID |
| | NO: 1 | NO: 34 | NO: 93 | NO: 15 | NO: 90 | ID | CH2-L2- | NO: 73 |
| | (IL2) | | (CH2) | | (IL-2Rα) | NO: 57 | IL-2Rα-LH | |
| 1023 | SEQ ID | SEQ ID | SEQ ID | SEQ ID | SEQ ID | SEQ | IL2-L1- | SEQ ID |
| | NO: 1 | NO: 2 | NO: 103 | NO: 35 | NO: 90 | ID | CH2-L2- | NO: 74 |
| | (IL2) | | (CH2) | | (IL-2Rα) | NO: 57 | IL-2Rα-LH | |
| 1025 | SEQ ID | SEQ ID | SEQ ID | SEQ ID | SEQ ID | SEQ | IL2-L1- | SEQ ID |
| | NO: 1 | NO: 2 | NO: 89 | NO: 15 | NO: 90 | ID | CH2CH3- | NO: 76 |
| | (IL2) | | (CH2CH3) | | (IL-2Rα) | NO: 57 | L2-IL-2Rα- | |
| | | | | | | | LH | |
| 1026 | SEQ ID | SEQ ID | SEQ ID | SEQ ID | SEQ ID | SEQ | IL2-L1- | SEQ ID |
| | NO: 1 | NO: 2 | NO: 91 | NO: 15 | NO: 90 | ID | CH2CH3- | NO: 77 |
| | (IL2) | | (CH2CH3) | | (IL-2Rα) | NO: 57 | L2-IL-2Rα- | |
| | | | | | | | LH | |
| 1027 | SEQ ID | SEQ ID | SEQ ID | SEQ ID | SEQ ID | SEQ | IL2-L1- | SEQ ID |
| | NO: 1 | NO: 22 | NO: 107 | NO: 15 | NO: 90 | ID | CH2-L2- | NO: 78 |
| | (IL2) | | (CH2) | | (IL-2Rα) | NO: 57 | IL-2Rα-LH | |
| 1028 | SEQ ID | SEQ ID | SEQ ID | SEQ ID | SEQ ID | SEQ | IL2-L1- | SEQ ID |
| | NO: 1 | NO: 2 | NO: 103 | NO: 18 | NO: 90 | ID | CH2-L2- | NO: 79 |
| | (IL2) | | (CH2) | | (IL-2Rα) | NO: 57 | IL-2Rα-LH | |
| 1029 | SEQ ID | SEQ ID | SEQ ID | SEQ ID | SEQ ID | SEQ | IL2-L1- | SEQ ID |
| | NO: 1 | NO: 36 | NO: 93 | NO: 15 | NO: 90 | ID | CH2-L2- | NO: 80 |
| | (IL2) | | (CH2) | | (IL-2Rα) | NO: 57 | IL-2Rα-LH | |
| 1030 | SEQ ID | SEQ ID | SEQ ID | SEQ ID | SEQ ID | SEQ | IL2-L1- | SEQ ID |
| | NO: 1 | NO: 37 | NO: 93 | NO: 15 | NO: 90 | ID | CH2-L2- | NO: 81 |
| | (IL2) | | (CH2) | | (IL-2Rα) | NO: 57 | IL-2Rα-LH | |
| 1031 | SEQ ID | SEQ ID | SEQ ID | SEQ ID | SEQ ID | SEQ | IL15-L1- | SEQ ID |
| | NO: 92 | NO: 37 | NO: 93 | NO: 15 | NO: 94 | ID | CH2-L2- | NO: 82 |
| | (IL15) | | (CH2) | | (IL-2Rα) | NO: 57 | IL-15Rα-LH | |
| 5001 | SEQ ID | SEQ ID | SEQ ID | SEQ ID | SEQ ID | | IL-2Rα-L1- | SEQ ID |
| | NO: 1 | NO: 19 | NO: 51 | NO: 24 | NO: 5 | | LC-L2-IL2 | NO: 53 |
| | (IL2) | | (LC) | | (IL-2Rα) | | | |
| 5002 | SEQ ID | SEQ ID | SEQ ID | SEQ ID | SEQ ID | | IL-2Rα-L1- | SEQ ID |
| | NO: 1 | NO: 19 | NO: 52 | NO: 24 | NO: 5 | | HC-L2-IL2 | NO: 54 |
| | (IL2) | | (HC) | | (IL-2Rα) | | | |
| 5003 | SEQ ID | SEQ ID | SEQ ID | SEQ ID | SEQ ID | | IL2-L1-LC- | SEQ ID |
| | NO: 1 | NO: 25 | NO: 51 | NO: 20 | NO: 5 | | L2-IL2-2Rα | NO: 55 |
| | (IL2) | | (LC) | | (IL-2Rα) | | | |
| 5004 | SEQ ID | SEQ ID | SEQ ID | SEQ ID | SEQ ID | | IL2-L1-HC- | SEQ ID |
| | NO: 1 | NO: 25 | NO: 52 | NO: 21 | NO: 5 | | L2-IL2-2Rα | NO: 56 |
| | (IL2) | | (HC) | | (IL-2Rα) | | | |

Additional exemplary cytokine receptor binding protein constructs are designed in accordance with the teachings herein to include the structural features as shown below in Table 3.

TABLE 3

| Construct | |
|---|---|
| ID | Description of additional designs of exemplary cytokine receptor binding protein |
| 1024 | In this design, an Fc binding peptide (SEQ ID NO: 32) is linked to the N-terminus of IL2 (SEQ ID NO: 1) |
| | through a third chemical linker (L3) (SEQ ID NO: 104); also included is an L1 (SEQ ID NO: 2), a |
| | CH2 (SEQ ID NO: 103), an L2 (SEQ ID NO: 15), an IL-2Rα (SEQ ID NO: 90), and an LH (SEQ ID |
| | NO: 57); the orientation of the design from N-to C-terminus is Fc binding domain-L3-IL2-L1-CH2- |
| | L2-IL-2Rα-LH. An exemplary amino acid sequence for this design includes the amino acid sequence |
| | of SEQ ID NO: 75. |
| 1032 | The exemplary occlusion domain in this design is a nanobody domain (SEQ ID NO: 95). Also included |
| | is IL2 (SEQ ID NO: 1), an L1 (SEQ ID NO: 2), an L2 (SEQ ID NO: 15), and an IL-2Rα (SEQ ID NO: 90). |
| | In one embodiment of this design, the orientation from N-to C-terminus is: IL2-L1-nanobody-L2- |
| | IL-2Rα. In another embodiment of this design, a linker with a histidine tag (SEQ ID NO: 57) is linked |
| | to the C-terminus of the IL-2Rα. An exemplary amino acid for this design includes the amino acid |
| | sequence of SEQ ID NO: 83. |
| 1033 | The exemplary occlusion domain in this design is a protein L domain (SEQ ID NO: 96). Also included |
| | is IL2 (SEQ ID NO: 1), an L1 (SEQ ID NO: 2), an L2 (SEQ ID NO: 15), and an IL-2Rα (SEQ ID NO: 90). |
| | In one embodiment of this design, the orientation from N-to C-terminus is: IL2-L1-protein L-L2- |
| | IL-2Rα. In another embodiment of this design, a linker with a histidine tag (SEQ ID NO: 57) is linked |
| | to the C-terminus of the IL-2Rα. An exemplary amino acid for this design includes the amino acid |
| | sequence of SEQ ID NO: 84. |
| 1034 | The exemplary occlusion domain in this design is a nanobody domain (SEQ ID NO: 97) that differs |
| | from the nanobody domain of construct 1032. Also included is IL2 (SEQ ID NO: 1), an L1 (SEQ ID NO: 2), |
| | an L2 (SEQ ID NO: 15), and an IL-2Rα (SEQ ID NO: 90). In one embodiment of this design, |
| | the orientation from N-to C-terminus is: IL2-L1-nanobody-L2-IL-2Rα. In another embodiment of this |
| | design, a linker with a histidine tag (SEQ ID NO: 57) is linked to the C-terminus of the IL-2Rα. An |
| | exemplary amino acid for this design includes the amino acid sequence of SEQ ID NO: 85. |
| 1035 | The exemplary occlusion domain in this design is a CD16 domain (SEQ ID NO: 98). Also included is |
| | IL2 (SEQ ID NO: 1), an L1 (SEQ ID NO: 2), an L2 (SEQ ID NO: 15), and an IL-2Rα (SEQ ID NO: 90). |
| | In one embodiment of this design, the orientation from N-to C-terminus is: IL2-L1-CD16-L2-IL-2Rα. |
| | In another embodiment of this design, a linker with a histidine tag (SEQ ID NO: 57) is linked to |
| | the C-terminus of the IL-2Rα. An exemplary amino acid for this design includes the amino acid |
| | sequence of SEQ ID NO: 86. |

As described and shown in Tables 2 and 3, some exemplary constructs include a linker with a histidine-tag, although this is optional and may be excluded in certain embodiments or variations of the construct. For instance, Table 4 provides exemplary amino acid sequences for variations of select constructs, as described in Tables 2 and 3, but lacking the linker with a histidine tag (SEQ ID NO: 57).

TABLE 4

| | | Exemplary sequence |
|---|---|---|
| | | of variant of |
| | Exemplary | construct lacking a |
| Construct | sequence | linker with a |
| ID | (from Table 2) | histidine tag |
| 329141 | SEQ ID NO: 38 | SEQ ID NO: 108 |
| 329141A | SEQ ID NO: 39 | SEQ ID NO: 109 |
| 329141B | SEQ ID NO: 40 | SEQ ID NO: 110 |
| 329141C | SEQ ID NO: 41 | SEQ ID NO: 111 |
| 329141D | SEQ ID NO: 42 | SEQ ID NO: 112 |
| 1015 | SEQ ID NO: 66 | SEQ ID NO: 119 |
| 1018 | SEQ ID NO: 69 | SEQ ID NO: 87 |
| 1019 | SEQ ID NO: 70 | SEQ ID NO: 118 |
| 1020 | SEQ ID NO: 71 | SEQ ID NO: 26 |
| 1021 | SEQ ID NO: 72 | SEQ ID NO: 27 |
| 1022 | SEQ ID NO: 73 | SEQ ID NO: 28 |
| 1023 | SEQ ID NO: 74 | SEQ ID NO: 30 |
| 1024 | SEQ ID NO: 75 | SEQ ID NO: 43 |
| 1025 | SEQ ID NO: 76 | SEQ ID NO: 44 |
| 1026 | SEQ ID NO: 77 | SEQ ID NO: 45 |
| 1027 | SEQ ID NO: 78 | SEQ ID NO: 46 |
| 1028 | SEQ ID NO: 79 | SEQ ID NO: 47 |
| 1029 | SEQ ID NO: 80 | SEQ ID NO: 48 |
| 1030 | SEQ ID NO: 81 | SEQ ID NO: 99 |
| 1031 | SEQ ID NO: 82 | SEQ ID NO: 113 |
| 1032 | SEQ ID NO: 83 | SEQ ID NO: 114 |
| 1033 | SEQ ID NO: 84 | SEQ ID NO: 115 |
| 1034 | SEQ ID NO: 85 | SEQ ID NO: 116 |
| 1035 | SEQ ID NO: 86 | SEQ ID NO: 117 |

DNA molecules encoding constructs are generated. Antibody expression is performed in expiCHO cells. Protein is purified to homogeneity and its purity is confirmed by SDS PAGE. Cleaved and uncleaved products can be characterized by mass spectrometry, SPR, and DSF.

### Example 2: In Vitro Analysis of Exemplary Cytokine Receptor Binding Proteins

Exemplary constructs are evaluated in vitro by assays, such as cell proliferation, ELISA, FACS, and in vitro activation assays. Pathways consistent with T cell activation (e.g., JAK/STAT activation) are measured.

The linker between IL2 and the occlusion domain (e.g., CH2 or Fab) is varied (shorter and longer) and evaluated in exemplary cytokine receptor binding proteins.

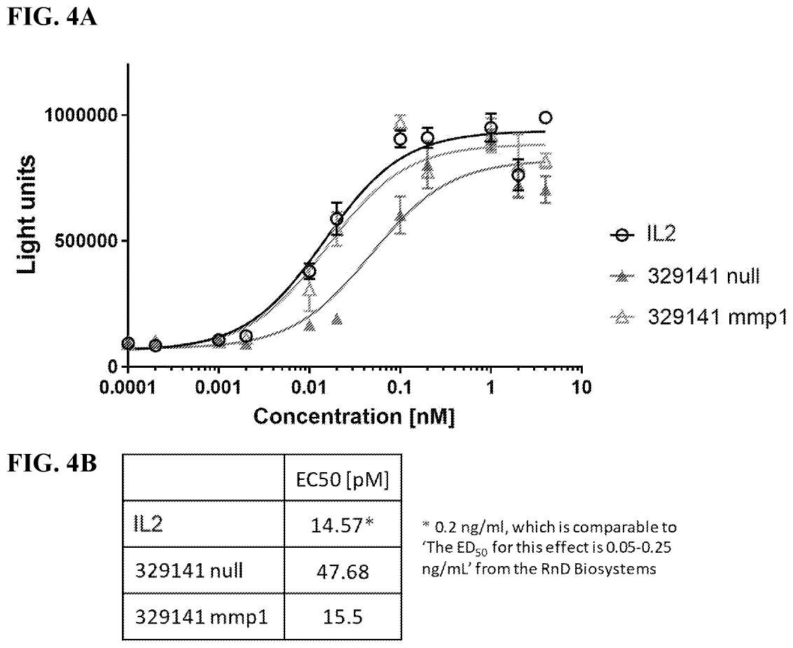

A cell proliferation assay was performed to compare the activity of a masked and activated form of the 329141 construct, including their activity relative to IL2 (from R& D Systems) as a control, using the mouse CTLL2 cytotoxic T cell line. FIG. 4A depicts the results of a cell proliferation assay that was performed by culturing the CellSensor® irfl-bla CTLL-2 cell line (from Invitrogen) in the presence of the 329141 construct that was previously exposed to the MMP1 protease (329141 mmp1; open triangle) or was not previously exposed to the MMP1 protease (329141 null; closed triangle). Exposure of the 329141 construct to MMP1 is expected to result in release of IL2 from the remainder of the construct (e.g., CH2 domain and IL-2Rα). The EC50 (pM) for each treatment was calculated and is depicted in FIG. 4B. As shown in FIG. 4B, the masked 329141 construct (329141 null) has an EC50 more than three times greater than the IL2 control, while the EC50 of the activated 329141 construct (329141 mmp1) is close to the EC50 of the IL2 control, as it is only approximately 7% higher than the EC50 of the IL2 control. The EC50 value of 14.57 pM for IL2 equates to approximately 0.20 ng/ml, which is comparable to ED50 for this effect, which, according to R&D Systems when describing this assay: “The ED50 for this effect is 0.05-0.25 ng/mL.” R&D Systems, Catalog Number 202-IL Product Datasheet.

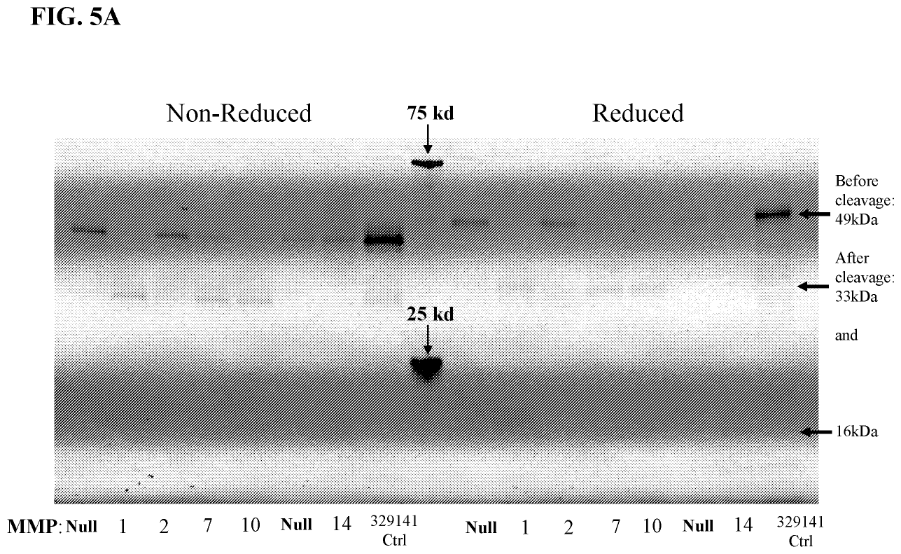

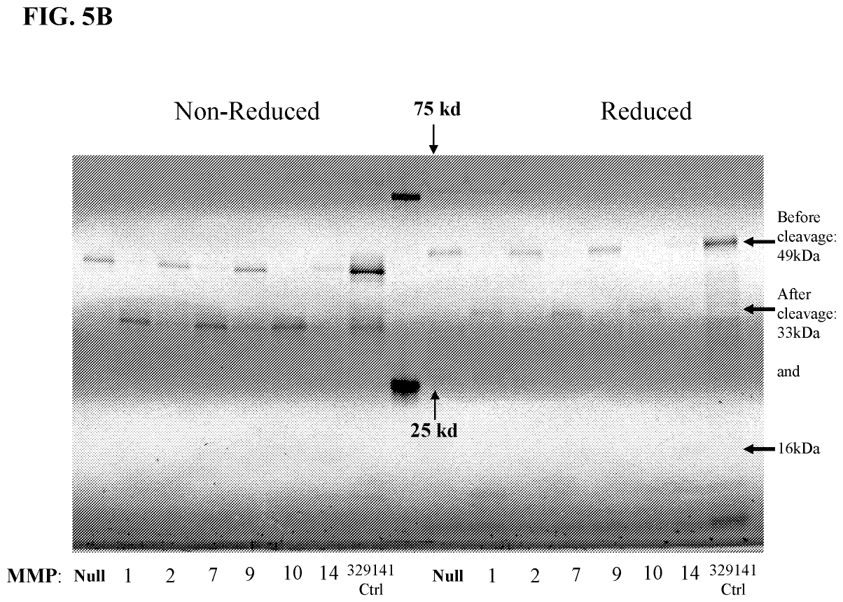

Cleavage of the exemplary 329141 construct by select proteases (MMP1, MMP2, MMP7, MMP9, MMP10, and MMP14) was assessed by incubating the construct in the presence of each protease overnight. SDS-PAGE analyses of the 329141 construct under non-reduced and reduced conditions are depicted in FIGS. 5A and 5B. MMPs 1, 2, 7, 10, and 14 were included in the analysis depicted in FIG. 5A, and MMPs 1, 2, 7, 9, 10, and 14 were included in the analysis depicted in FIG. 5B. As shown in FIGS. 5A and 5B, the 329141 construct was cleaved by MMP1, MMP7, and MMP10.

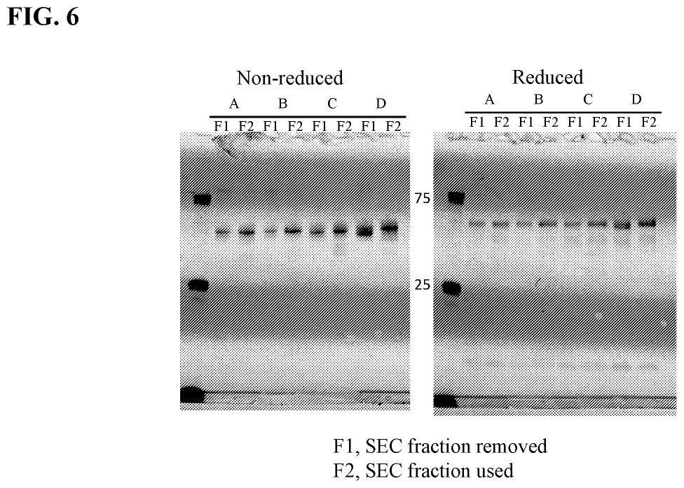

As shown in FIG. 6, the 329141A (“A”), 329141B (“B”), 329141C (“C”), and 329141D (“D”) constructs were analyzed using SDS-PAGE analysis under non-reduced (left) and reduced (right) conditions using samples where the size-exclusion chromatography (SEC) fraction was removed (“F1”) or used (“F2”).

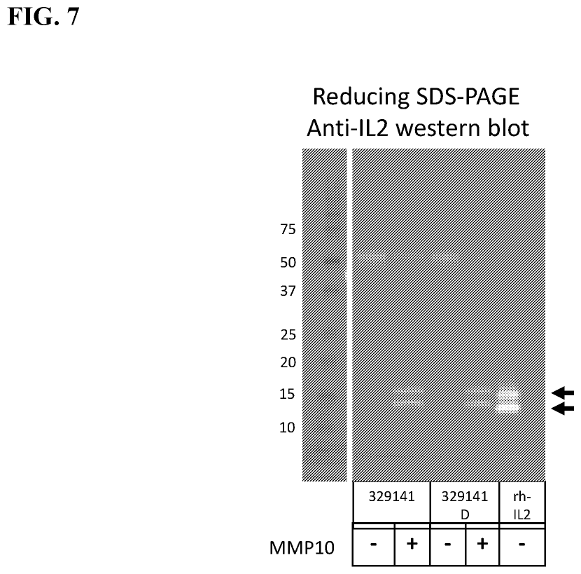

Activation of the exemplary 329141 and 329141D constructs were also tested using MMP10 as the activating protease. The cytokine receptor binding protein of interest (329141 or 329141D) was added to MMP cleavage buffer containing 50 mM Tris pH 7.4, 150 mM sodium chloride, and 10 mM calcium chloride. MMP10 was activated using 1 mM APMA for 2 hours at 37 degrees C. Samples were incubated overnight at 37 degrees C. in the presence or absence of activated MMP10. MMP10 was added to the MMP cleavage buffer at a 1:500 ratio (protease:protein) for the samples incubated in the presence of activated MMP10. As a control, recombinant human IL2 (rhIL2) was also tested, which involved adding rhIL2 to MMP cleavage buffer and incubating it in the absence of MMP10. As shown in FIG. 7, the 329141 and 329141D constructs were cleaved in the presence of MMP10, which resulted in the release of IL2.

Binding of an exemplary cytokine receptor binding protein, 329141, to CD25 and to CD122 was assessed using surface plasmon resonance (SPR). The 329141 construct was immobilized to a sensor chip at a concentration of 30 mg/mL in 10 mM sodium acetate pH 5.0. As a control, rhIL2 was immobilized to a sensor chip at a concentration of 30 mg/mL in 10 mM sodium acetate pH 5.0. rhCD25-Fc was used as analyte at concentrations of 16 nM, 8 nM, 4 nM, 2 nM, and 1 nM. rhFC-CD122 was used as analyte at concentrations of 500 nM, 250 nM, 125 nM, 62.5 nM, and 31.25 nM.

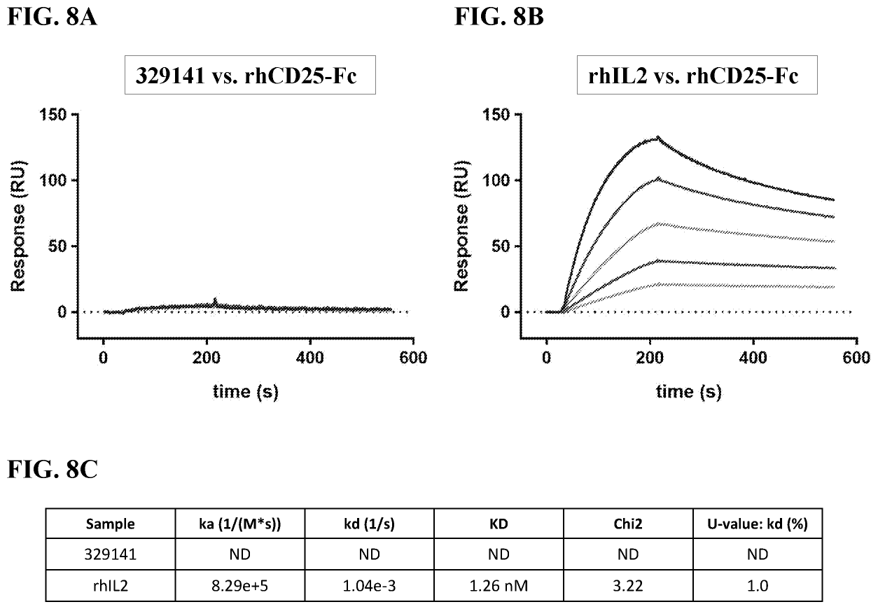

Results from the SPR analysis using rhCD25-Fc as analyte are depicted in FIGS. 8A-8C. FIG. 8A depicts the interaction between the 329141 construct and rhCD25-Fc, FIG. 8B depicts the interaction between rhIL2 and rhCD25-Fc, and FIG. 8C provides data on the association rate (ka), dissociation rate (kd), and equilibrium dissociation constant (KD), as well as Chi² and U-values for the interaction between rhCD25-Fc and either the 329141 construct or rhIL2, as determined in this analysis. As shown in FIG. 8C, although binding was detected between rhIL2 and rhCD25-Fc (the positive control), no binding was detected between the masked 329141 construct and rhCD25-Fc.

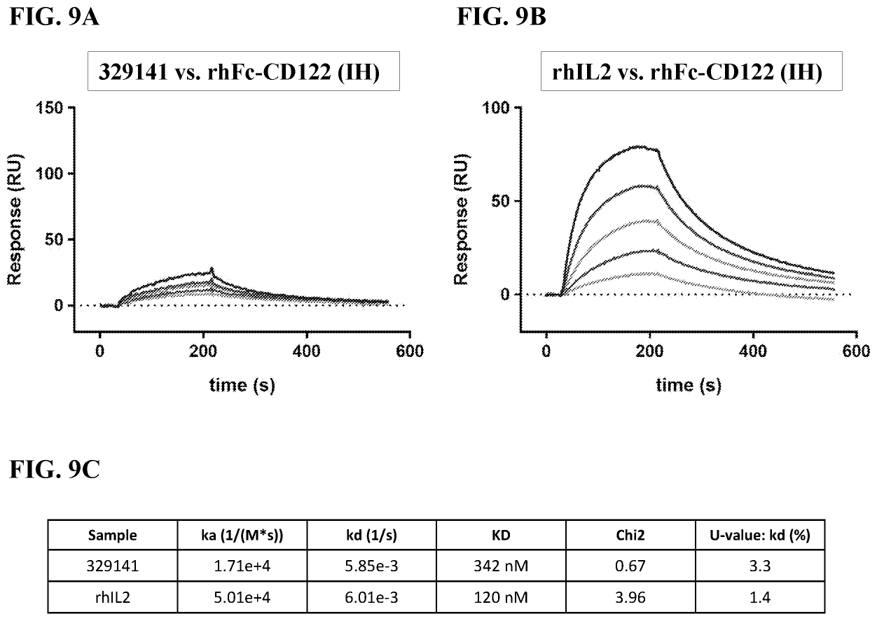

Results from the SPR analysis using rhFc-CD122 as analyte are depicted in FIGS. 9A-9C. FIG. 9A depicts the interaction between the 329141 construct and rhFc-CD122, FIG. 9B depicts the interaction between rhIL2 and rhFc-CD122, and FIG. 9C provides data on the association rate (ka), dissociation rate (kd), and equilibrium dissociation constant (KD), as well as Chi² and U-values for the interaction between rhFc-CD122 and either the 329141 construct or rhIL2, as determined in this analysis. As shown in FIG. 9C, the masked 329141 construct demonstrates reduced binding to rhFc-CD122 as compared to the rhIL2 control.

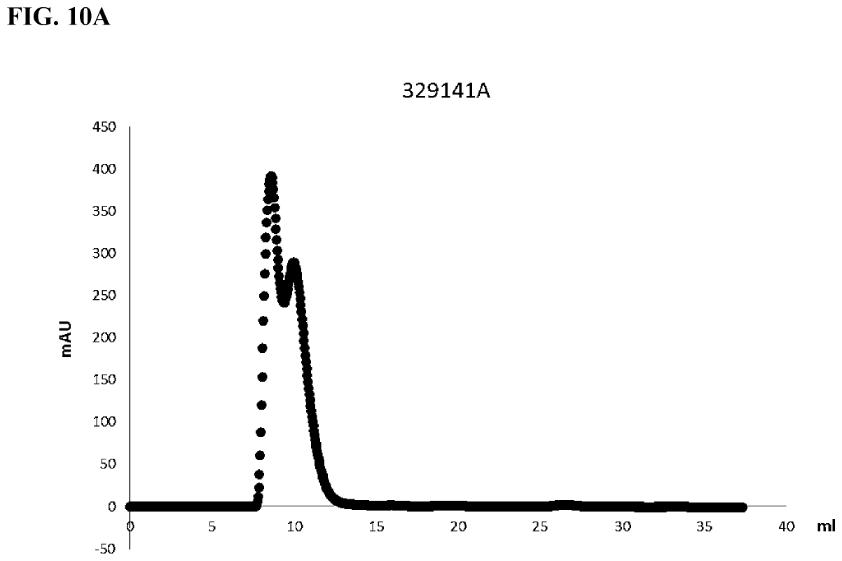

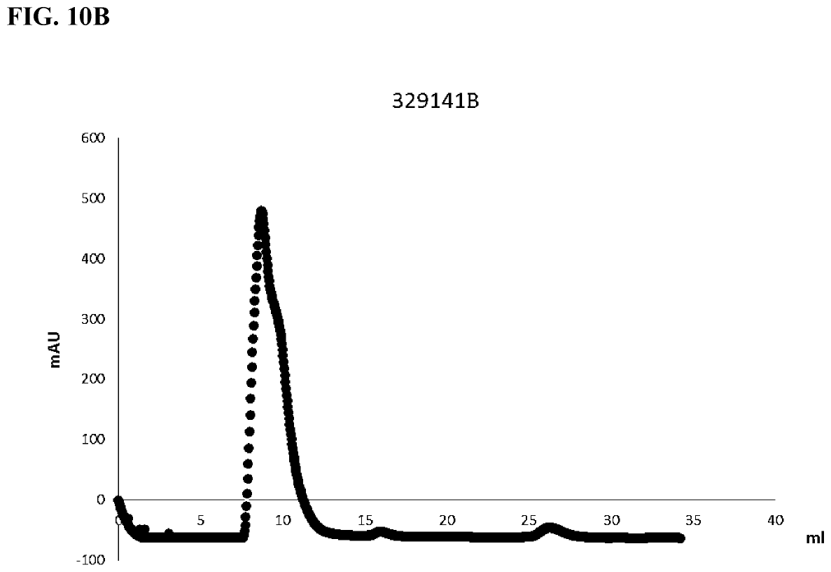

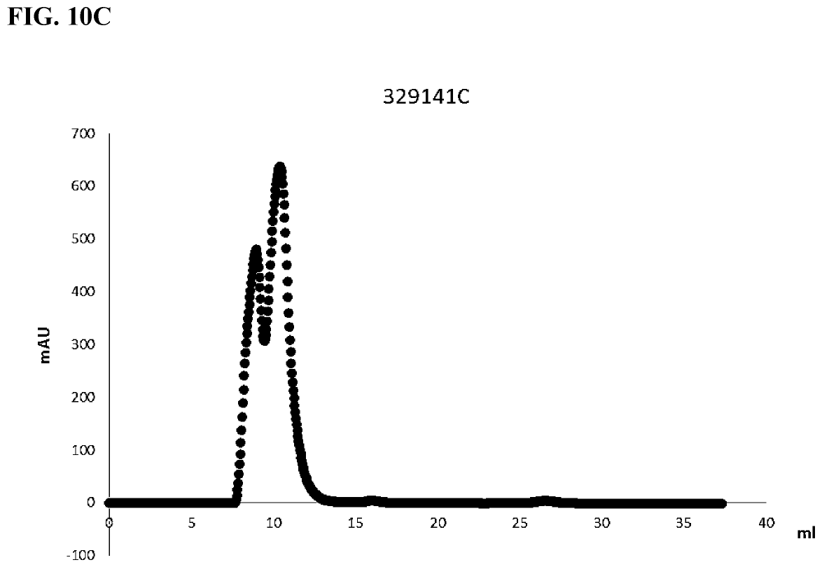

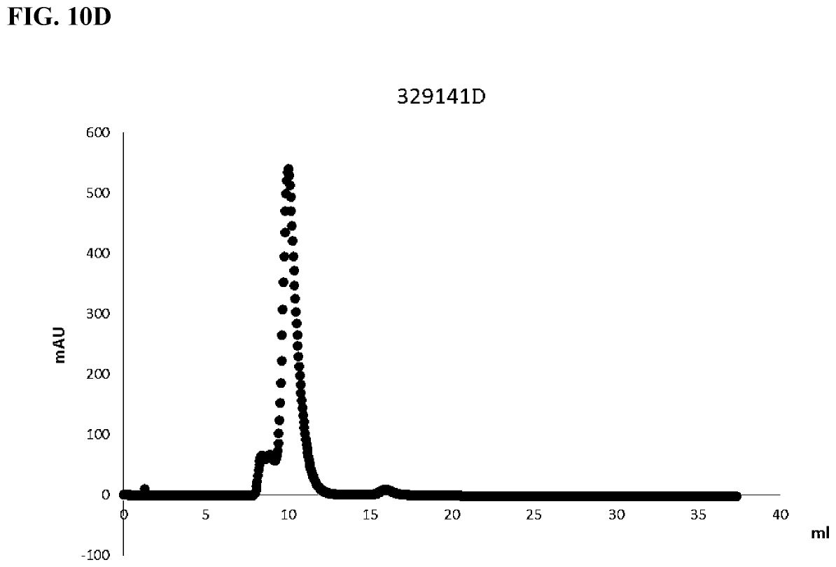

An analysis of the 329141A, 329141B, 329141C, and 329141D constructs using size exclusion chromatograph (SEC) is depicted in FIGS. 10A-10D.

An SDS-PAGE analysis revealed that each of the 329141A, 329141B, 329141C, and 329141D constructs were cleaved by MMP7, and that the 329141C and 329141D constructs exhibited almost complete cleavage by MMP1, MMP7, and MMP10 (data not shown).

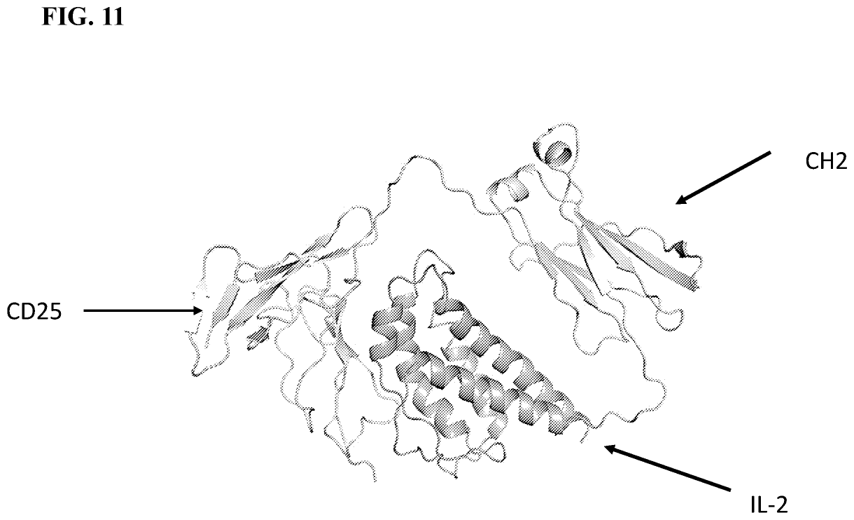

FIG. 11 is a 3D modeling depiction of the exemplary 329141 construct, with arrows pointing to a CH2 domain, IL-2 domain, and CD25 (IL-2Rα) domain.

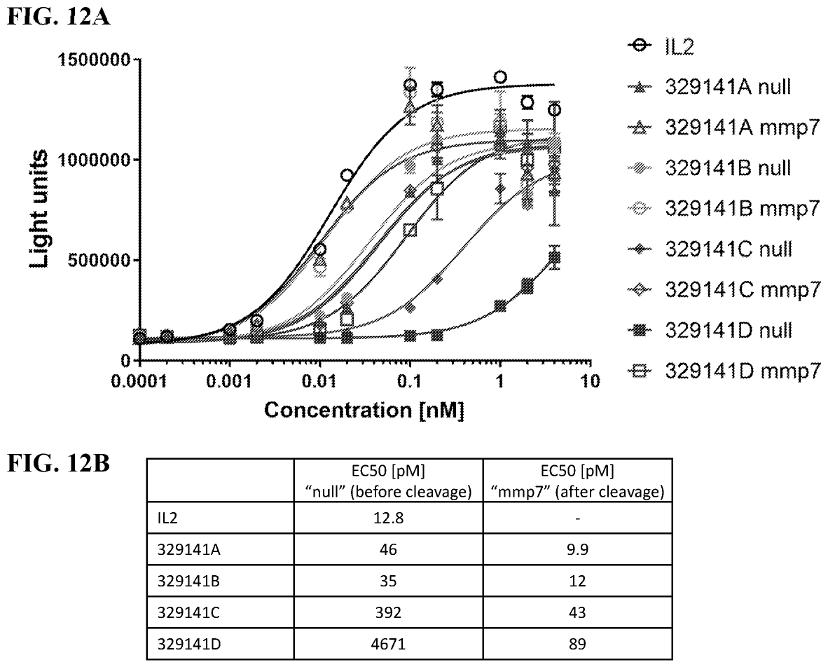

A cell proliferation assay was performed to compare the activity of a masked and activated form of the 329141A, 329141B, 329141C, and 329141D constructs, including their activity relative to IL2 (from R& D Systems) as a control, using the mouse CTLL2 cytotoxic T cell line. FIG. 12A depicts the results of a cell proliferation assay that was performed by culturing the CellSensor® irfl-bla CTLL-2 cell line (from Invitrogen) in the presence of each construct that was previously exposed to the MMP7 protease (mmp7; open symbol) or was not previously exposed to the MMP7 protease (null; closed symbol). Exposure of the constructs to MMP7 is expected to result in release of IL2 from the remainder of the construct (e.g., CH2 domain and IL-2Rα). The EC50 (pM) of each treatment was calculated and is depicted in FIG. 12B. As shown in FIG. 12B, each of the masked constructs have an EC50 that is greater than the EC50 of the IL2 control and is reduced by cleavage with MMP7. FIG. 12B also shows that the masked 329141D construct (null; before cleavage) has an EC50 that is more than 50 times greater than the EC50 of the activated 329141D construct (mmp7; after cleavage). This data demonstrates that there is strong inhibition of IL2 activity in the masked 329141D construct, which can be abrogated upon activation by a cleaving protease, such as MMP7.

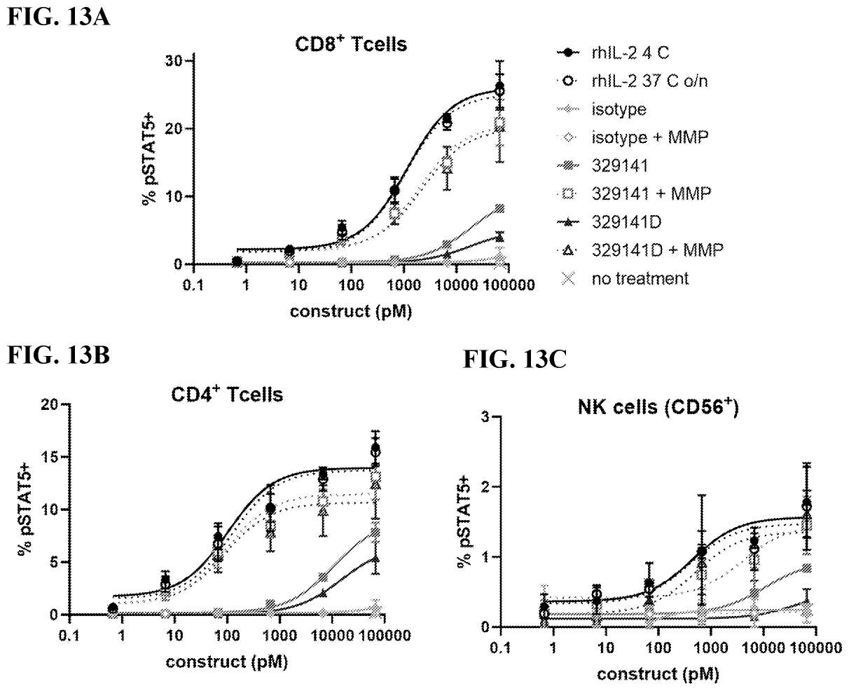

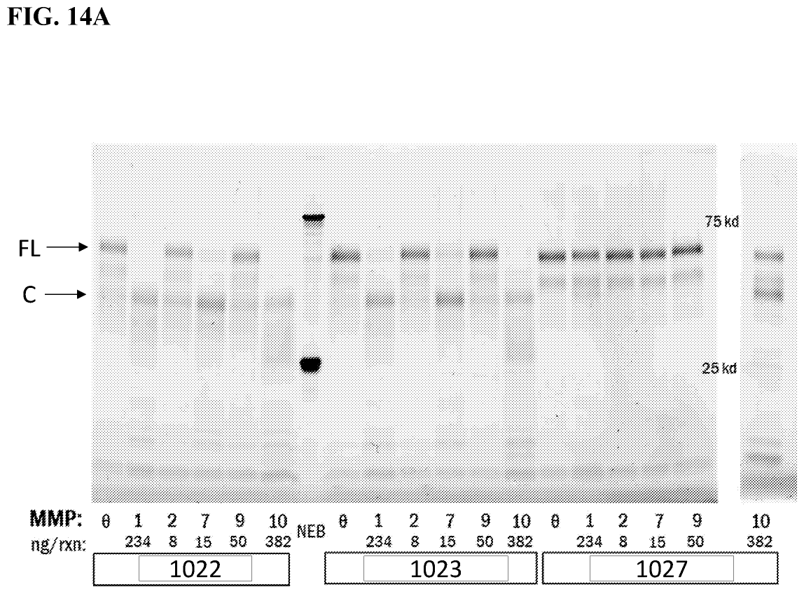

Stat5 activation assays were performed using human peripheral blood lymphocytes, including CD8+ T cells, CD4+ T cells, and Natural Killer (NK) cells, exposed to a masked (closed symbol) or activated (open symbol) form of the 329141 and 329141D constructs, as well as rhIL2 as a positive control, an isotype control, and a no treatment control. The masked form of the construct lacked previous exposure to a cleaving protease (e.g., MMP10), and the activated form of the construct was previously exposed to the cleaving protease MMP10. The results are depicted in FIGS. 13A-13C, which show the percentage of phosphorylated Stat5 (pStat5) positive CD8+ T cells (FIG. 13A), CD4+ T cells (FIG. 13B), and NK cells (FIG. 13C) when various concentrations of the construct or rhIL2 are exposed to the cells. The results demonstrate that activation of the 329141 and 329141D constructs by protease cleavage promotes Stat5 activation, which is reduced prior to protease cleavage as compared to the masked construct and the rhIL2 control. The legend shown in FIG. 13A also applies to FIGS. 13B and 13C.

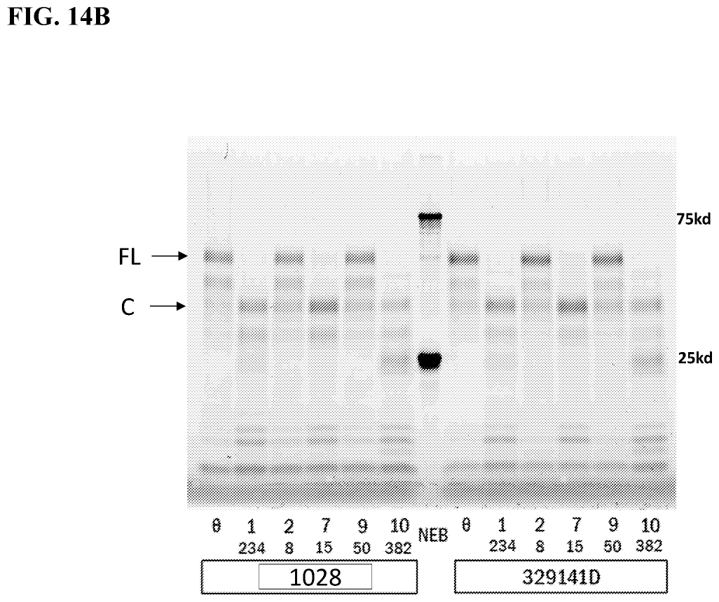

Cleavage of the exemplary constructs 1022, 1023, 1027, 1028, and 329141D by select proteases (MMP1, MMP2, MMP7, MMP9, MMP10) was assessed by incubating each construct (1000 nM protein per reaction) in the presence of each protease, or in the absence of protease as a control (0), overnight. The 1022 construct, as described above, is a variant of the 329141D construct and includes an Fc binding peptide situated at the N-terminus of the CH2, the 1023 construct, as described above, is a variant of the 329141D construct that includes an Fc binding peptide situated at the C-terminus of the CH2, and the 1028 construct, as described above, is a variant of the 329141D construct due to modifying the sequence of the second chemical linker. The 1027 construct is a variant of the 329141D construct that is non-cleavable due to an alteration of the first chemical linker sequence. SDS-PAGE analyses of the constructs under reduced conditions are shown in FIGS. 14A and 14B, with the arrows indicating the expected size of the full-length (FL) construct (i.e., uncleaved) and the cleaved construct (C) following release of the cytokine domain (IL2) by protease cleavage. FIGS. 14A and 14B reveal that the cleavable constructs (1022, 1023, 1028, and 329141D) were cleaved by MMP1, MMP7, and MMP10.

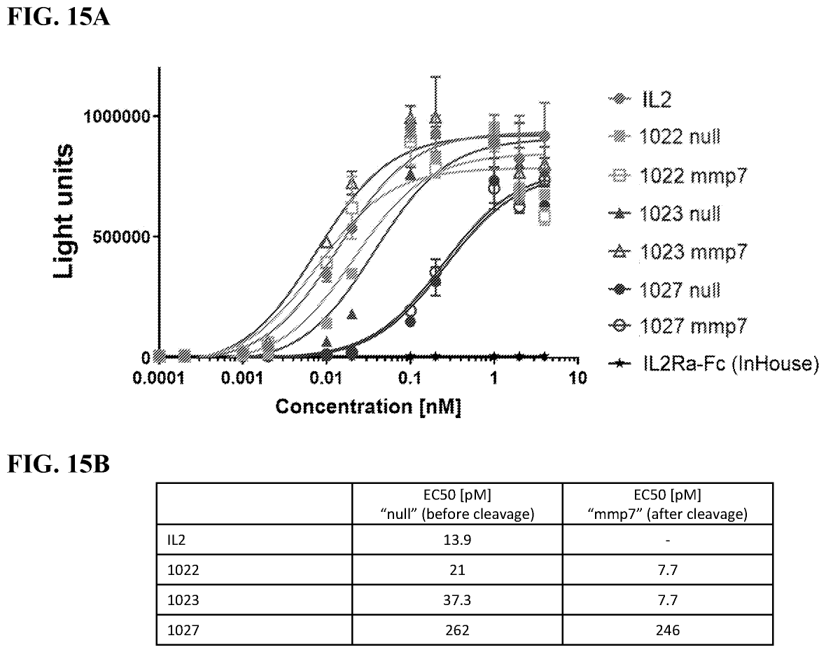

A cell proliferation assay was performed to compare the activity of a masked and activated form of the 1022, 1023, and 1027 constructs, including their activity relative to IL2 (from R& D Systems) as a positive control, using the mouse CTLL2 cytotoxic T cell line, as previously described. IL2Rα-Fc was also included as a negative control. FIG. 15A depicts the results of a cell proliferation assay where the cell line was cultured in the presence of each construct that was previously exposed to the MMP7 protease (mmp7; open symbol) or was not previously exposed to the MMP7 protease (null; closed symbol). The EC50 (pM) of each treatment was calculated and is depicted in FIG. 15B. As shown in FIG. 15B, each of the masked constructs have an EC50 that is greater than the EC50 of the IL2 control, and there is a decrease in the EC50 of the 1022 and 1023 constructs following exposure to the MMP7 protease, as compared to the construct before cleavage.

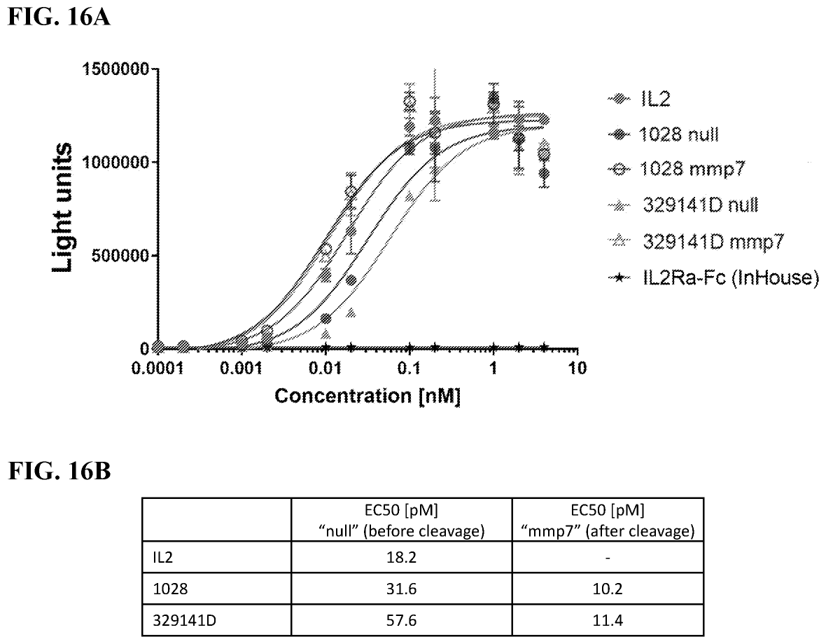

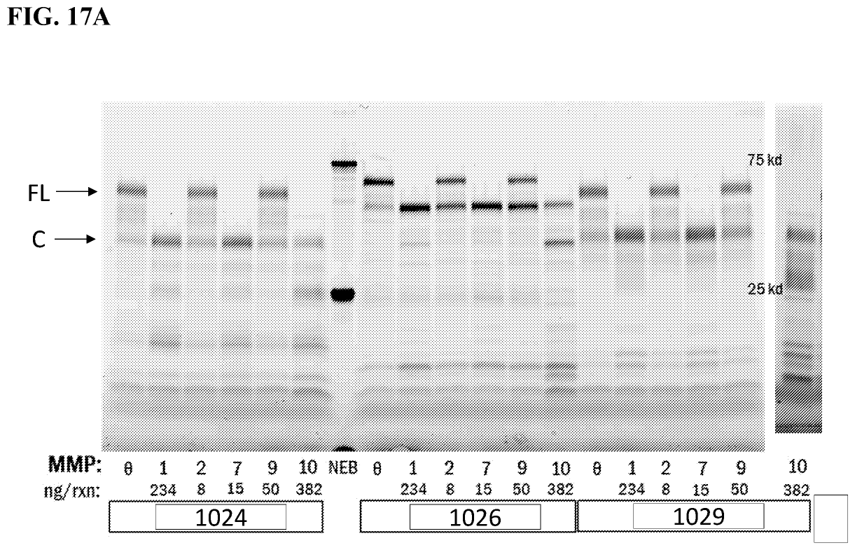

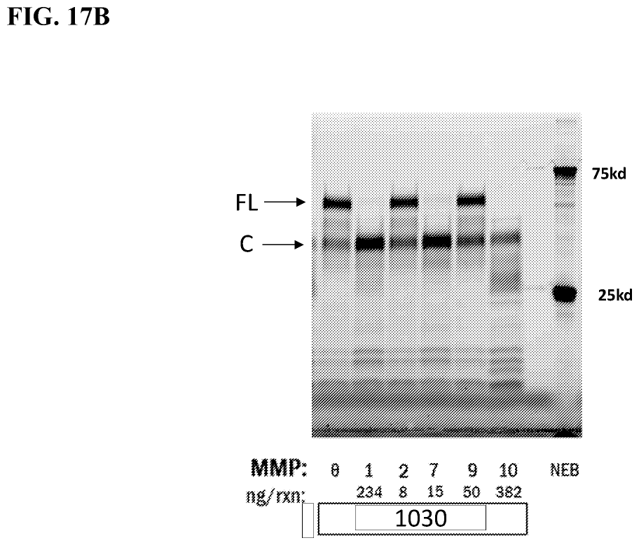

A cell proliferation assay was performed to compare the activity of a masked and activated form of the 1028 and 329141D constructs, including their activity relative to IL2 (from R& D Systems) as a positive control, using the mouse CTLL2 cytotoxic T cell line, as previously described. IL2Rα-Fc was also included as a negative control. FIG. 16A depicts the results of a cell proliferation assay where the cell line was cultured in the presence of each construct that was previously exposed to the MMP7 protease (mmp7; open symbol) or was not previously exposed to the MMP7 protease (null; closed symbol). The EC50 (pM) of each treatment was calculated and is depicted in FIG. 16B. As shown in FIG. 16B, each of the masked constructs have an EC50 that is greater than the EC50 of the IL2 control, and there is a decrease in the EC50 of the 1028 and 329141D constructs following exposure to the MMP7 protease, as compared to the construct before cleavage. Cleavage of the exemplary constructs 1024, 1026, 1029, and 1030 by select proteases (MMP1, MMP2, MMP7, MMP9, MMP10) was assessed by incubating each construct (1000 nM protein per reaction) in the presence of each protease, or in the absence of protease as a control (0), overnight. The 1024 construct, as described above, is a variant of the 329141D construct that includes an Fc binding peptide situated at the N-terminus of the IL-2. The 1026 construct, as described above, includes a monomeric CH2CH3 domain as the occlusion domain. The 1029 and 1030 constructs, as described above, are variants of the 329141D construct that each include an Fc binding peptide situated at the N-terminus of the CH2. SDS-PAGE analyses of the constructs under reduced conditions are shown in FIGS. 17A and 17B, with the arrows indicating the expected size of the full-length (FL) construct (i.e., uncleaved) and the cleaved construct (C) following release of the cytokine domain (IL2) by protease cleavage. FIGS. 17A and 17B reveal that each of the constructs (1024, 1026, 1029, and 1030) were cleaved by MMP1, MMP7, and MMP10.

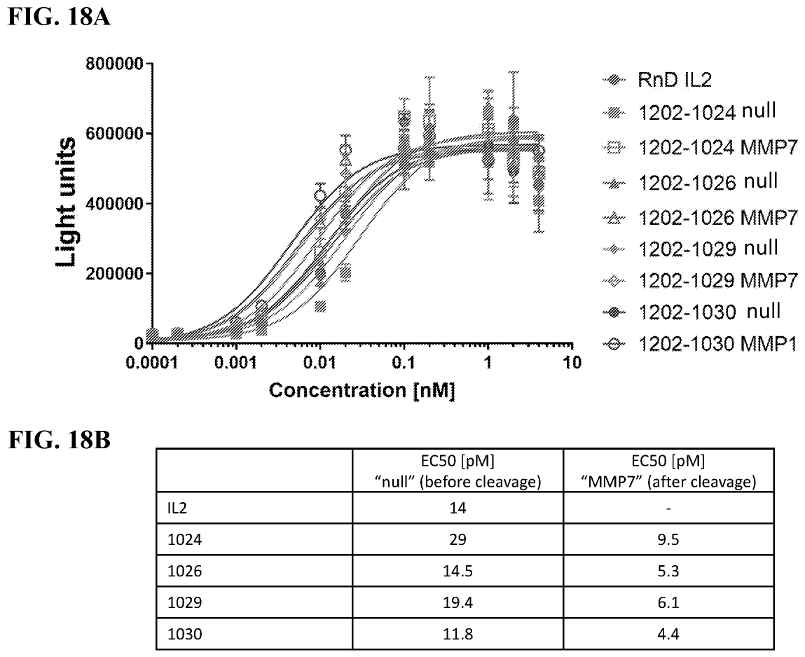

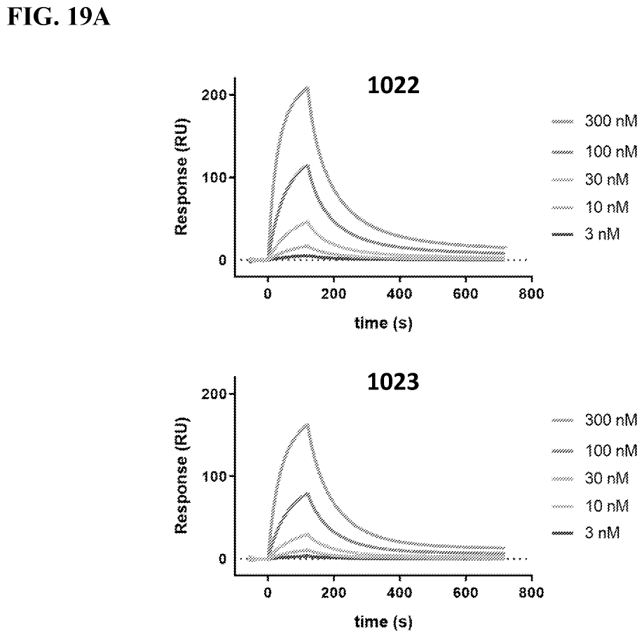

A cell proliferation assay was performed to compare the activity of a masked and activated form of the exemplary 1024, 1026, 1029, and 1030 constructs, including their activity relative to IL2 (from R& D Systems) as a positive control, using the mouse CTLL2 cytotoxic T cell line, as previously described. FIGS. 18A and 18B depict the results of a cell proliferation assay where the cell line was cultured in the presence of each construct (1024, 1026, 1029, or 1030) that was previously exposed the MMP7 protease (MMP7 or MMP1; open symbol) or was not previously exposed to the MMP7 or MMP1 protease (null; closed symbol). The EC50 (pM) of each treatment was calculated and is depicted in FIG. 18B. As shown in FIG. 18B, there is a decrease in the EC50 of each of the constructs (1024, 1026, 1029, and 1030) following exposure to the MMP7 or MMP1 protease, as compared to the construct before cleavage.

Binding of exemplary constructs that contain an Fc binding peptide to a humanized Fc-containing IgG1 antibody was assessed using SPR. The exemplary constructs 1022, 1023, 1024, 1029, and 1030, each of which include an Fc binding peptide, were tested for their binding to an exemplary humanized Fc-containing IgG1 antibody, trastuzumab. Trastuzumab was immobilized on a CM5 chip (1000 resonance units (RU)) through EDC/NHS coupling. Each of these exemplary constructs (1022, 1023, 1024, 1029, and 1030) was also tested for its ability to bind mouse IgG2a, by performing studies in which mouse IgG2a was immobilized on a CM5 chip (1000 RU) through EDC/NHS coupling. For both sets of experiments, each of the construct tested (1022, 1023, 1024, 1029, and 1030) were prepared in HBS-EP+ running buffer and were used as analyte at concentrations of 300 nM, 100 nM, 30 nM, 10 nM, and 3 nM.

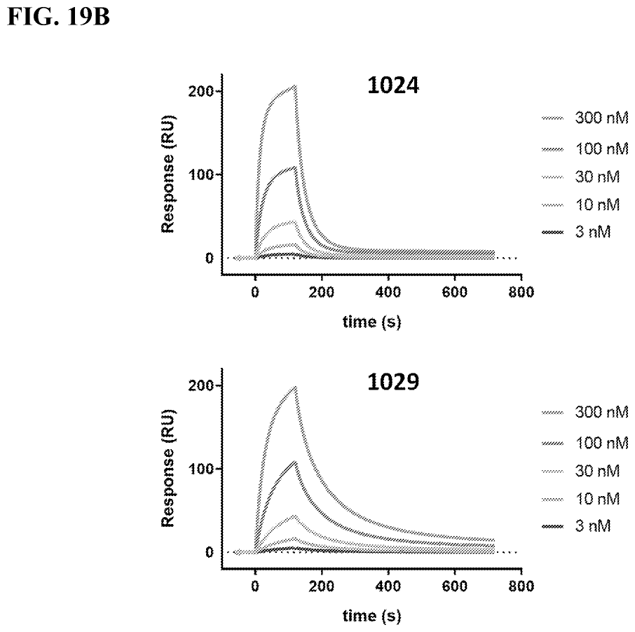

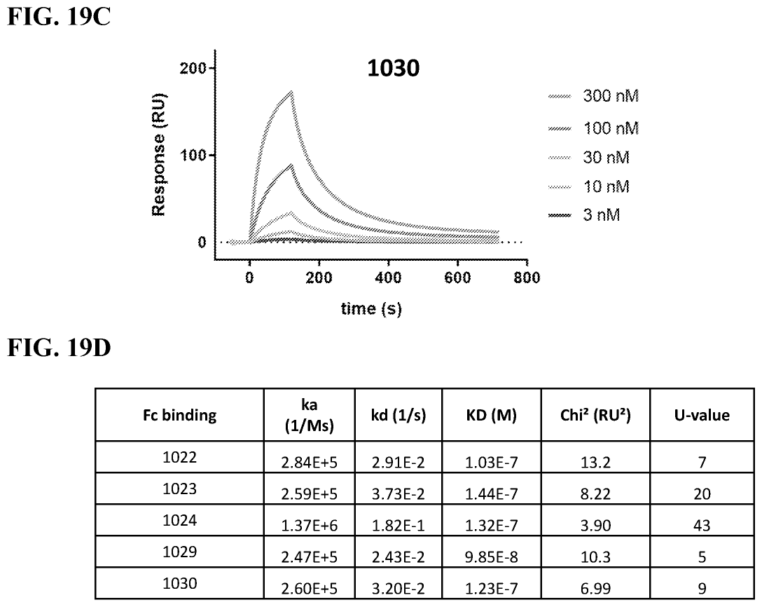

Results from the SPR analyses testing binding of the 1022, 1023, 1024, 1029, and 1030 constructs to trastuzumab (a humanized IgG1 antibody) are depicted in FIGS. 19A-19D. FIGS. 19A-19C depict the interaction between each of the 1022, 1023, 1024, 1029, and 1030 constructs and the Fc-containing antibody trastuzumab. FIG. 19D provides data on the association rate (ka), dissociation rate (kd), and equilibrium dissociation constant (KD), as well as Chi² and U-values for the interaction between each construct and trastuzumab, as determined in this analysis. As shown in FIGS. 19A-19D, each of the constructs tested was shown to bind to the humanized Fc-containing antibody trastuzumab.

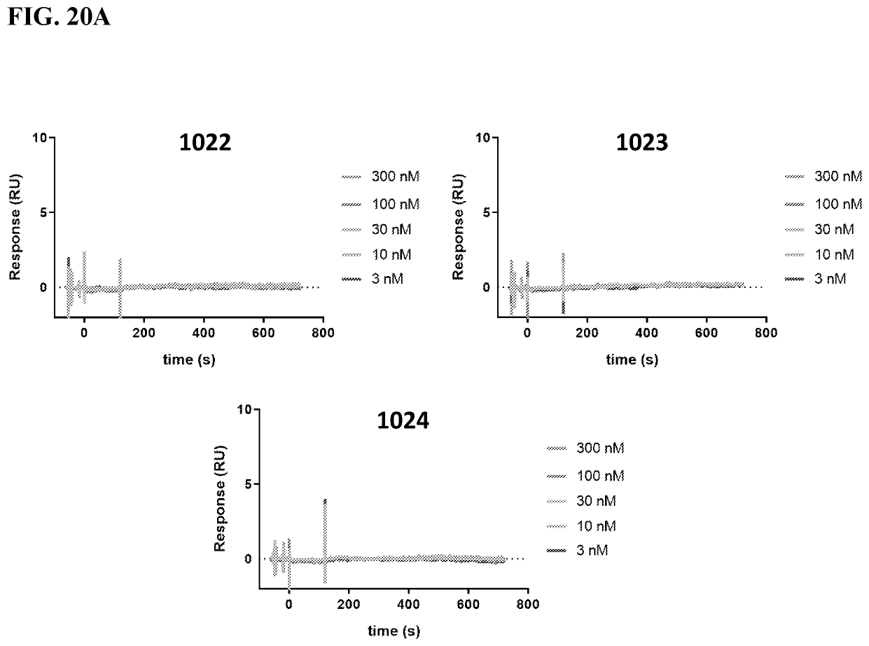

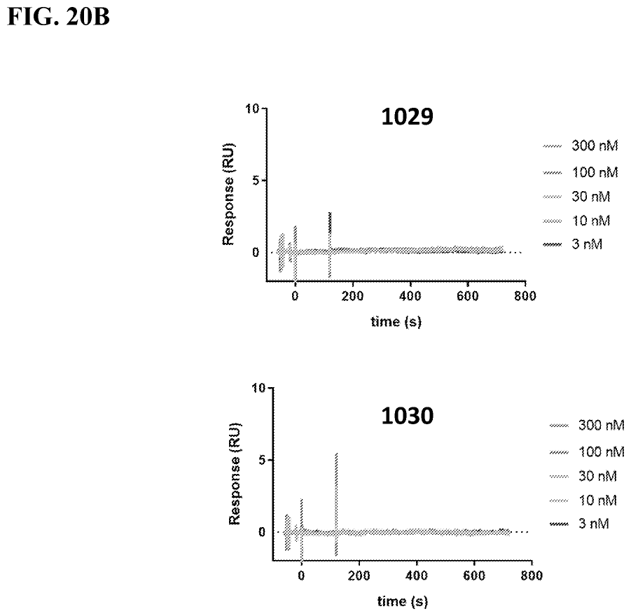

Results from the SPR analyses testing binding of the 1022, 1023, 1024, 1029, and 1030 constructs to mouse IgG2a are depicted in FIGS. 20A and 20B, which shows that these constructs did not bind to mouse IgG2a.

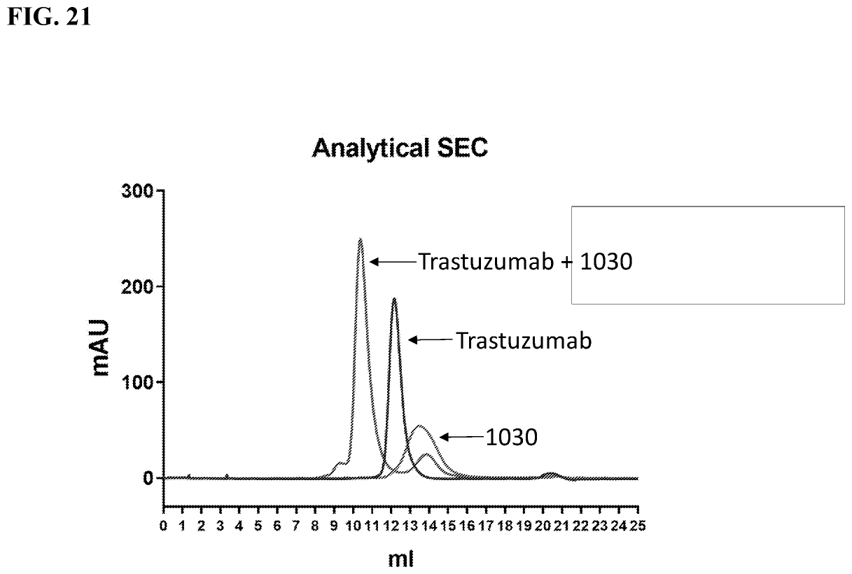

SEC analysis was performed on the 1030 construct, a clinical humanized IgG1 antibody (trastuzumab), and a combination of the 1030 construct and trastuzumab at a 1:1 molar ratio for one hour at room temperature prior to analysis. Results from the SEC analyses is depicted in FIG. 21, which demonstrated that the 1030 construct and trastuzumab were primarily in complex with one another.

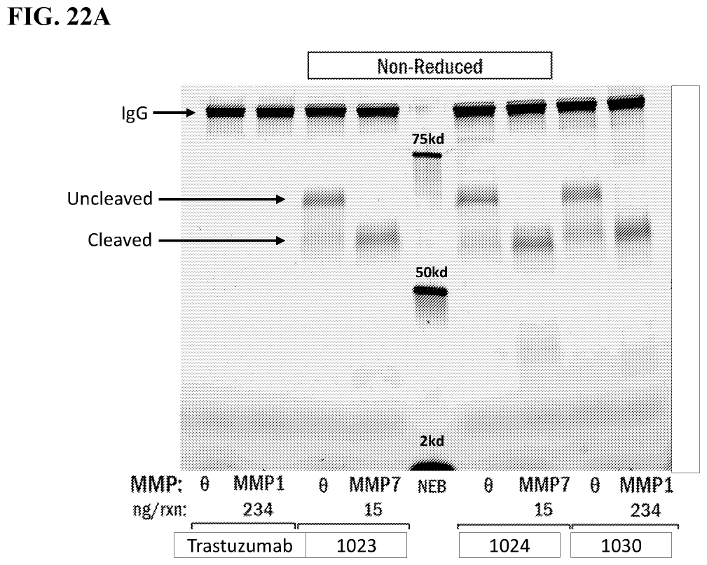

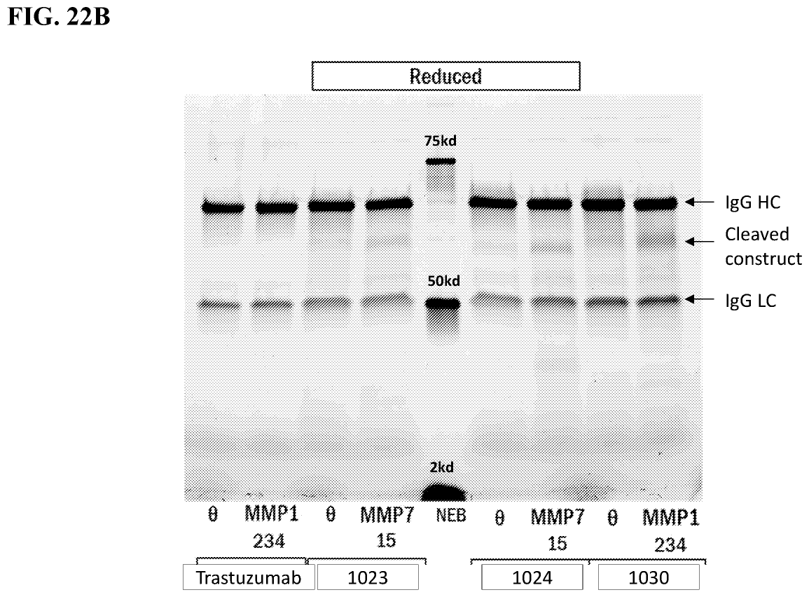

Cleavage of the exemplary constructs 1023, 1024, and 1030 by MMP1 or MMP7 while in complex with an IgG was assessed by combining trastuzumab with each construct (1000 nM protein per reaction) at a 1:1 molar ratio and incubating the mixture in the presence of protease (MMP1 or MMP7), or in the absence of protease as a control (0), overnight. As a control, trastuzumab was also individually incubated in the presence of MMP1 or in the absence of protease (0). SDS-PAGE analyses of the constructs under non-reduced and reduced conditions are shown in FIGS. 22A and 22B, respectively. As shown in FIGS. 22A and 22B, the presence of an IgG does not interfere with cleavage by exemplary MMPs, which demonstrates that cleavage can still occur while a cytokine receptor binding protein that includes an Fc binding peptide is in complex with an IgG.

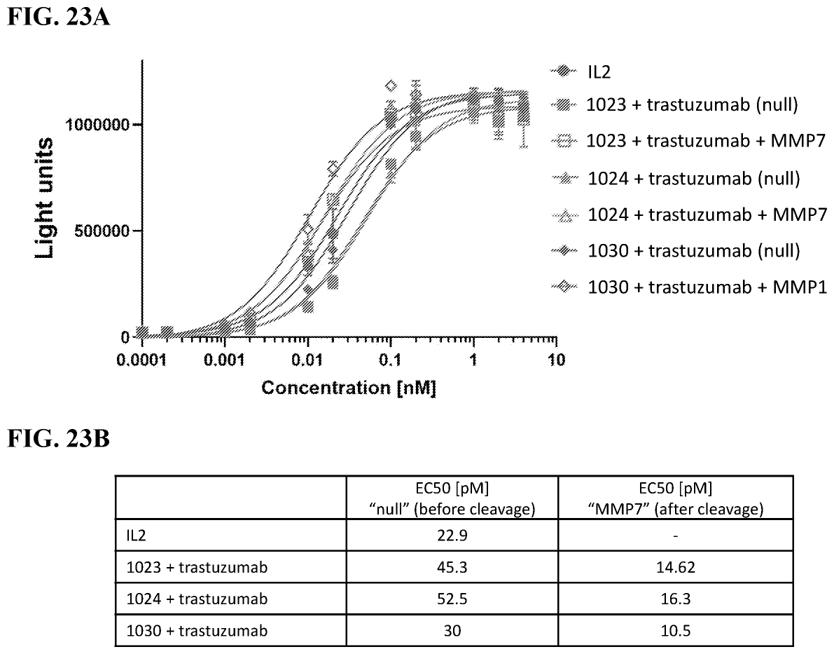

A cell proliferation assay was performed to compare the activity of a masked and activated form of the 1023, 1024, and 1030 constructs in the presence of an IgG, including their activity relative to IL2 (from R& D Systems) as a positive control, using the mouse CTLL2 cytotoxic T cell line, as previously described. FIGS. 23A and 23B depict the results of a cell proliferation assay where the cell line was cultured in the presence of each construct (1023, 1024, or 1030) that was previously exposed the MMP7 protease (MMP7; open symbol) or was not previously exposed to the MMP7 protease (null; closed symbol), while in the presence of an IgG (trastuzumab). The EC50 (pM) of each treatment was calculated and is depicted in FIG. 23B. As shown in FIG. 23B, each of the masked constructs have an EC50 that is greater than the EC50 of the IL2 control, and there is a decrease in the EC50 of the 1023, 1024, and 1030 constructs following exposure to the MMP7 protease, as compared to the construct before cleavage, which demonstrates that the presence of an exemplary IgG (trastuzumab) does not interfere with the activity of activated forms of the exemplary 1023, 1024, and 1030 constructs. For a comparison of the decrease in the EC50 of the 1023, 1024, and 1030 constructs when in the presence of trastuzumab to the decrease in the EC50 of these constructs tested in the absence of trastuzumab, compare FIG. 23B to FIGS. 15B and 18B.

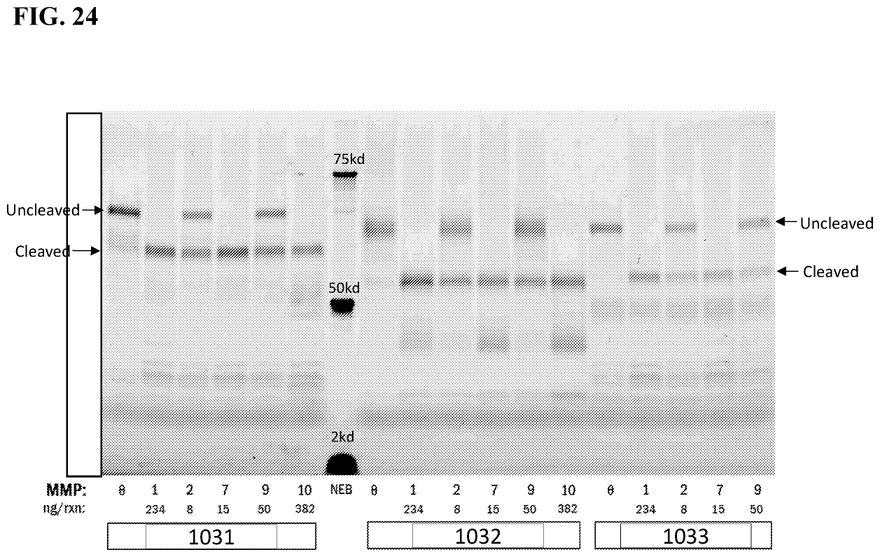

Cleavage of the exemplary constructs 1031, 1032, and 1033 by select proteases (MMP1, MMP2, MMP7, MMP9, MMP10) was assessed by incubating each construct (1000 nM protein per reaction) in the presence of each protease, or in the absence of protease as a control (0), overnight. The 1031 construct, as described above, is a masked IL15 construct that includes an Fc binding peptide at the N-terminus of the CH2. The 1032 construct, as described above, is a masked IL2 construct that includes a nanobody domain as the occlusion domain. The 1033 construct, as described above, is a masked IL2 construct that includes a protein L (proL) domain as the occlusion domain. SDS-PAGE analysis of the constructs under reduced conditions is shown in FIG. 24, with the arrows indicating the expected size of the uncleaved construct and the cleaved construct following release of the cytokine domain (IL2) by protease cleavage. FIG. 24 reveals that each of the constructs (1031, 1032, and 1033) were cleaved by each of the proteases (MMP1, MMP2, MMP7, MMP9, and MMP10), with MMP1, MMP7, and MMP10 demonstrating the most complete cleavage efficiency.

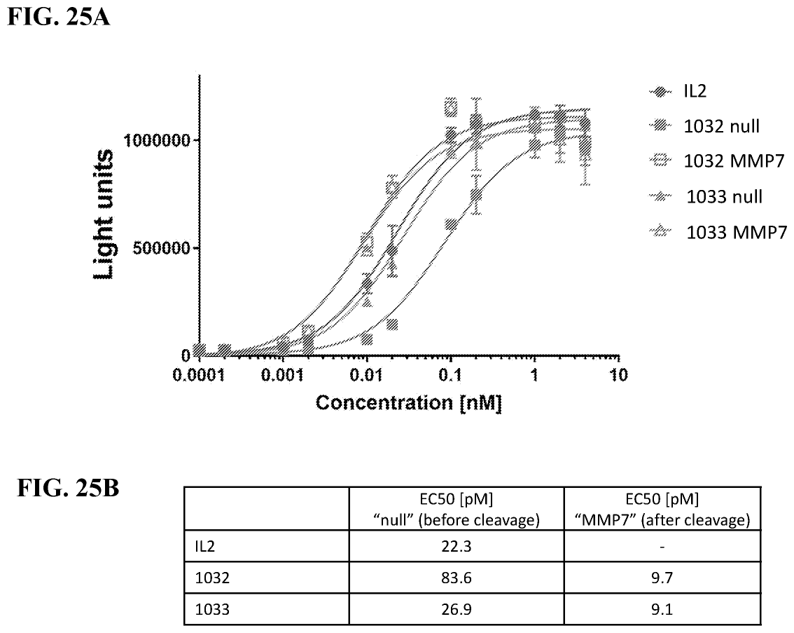

A cell proliferation assay was performed to compare the activity of a masked and activated form of the 1032 and 1033 constructs, including their activity relative to IL2 (from R& D Systems) as a positive control, using the mouse CTLL2 cytotoxic T cell line, as previously described. FIGS. 25A and 25B depict the results of a cell proliferation assay where the cell line was cultured in the presence of each construct (1032 or 1033) that was previously exposed the MMP7 protease (MMP7; open symbol) or was not previously exposed to the MMP7 protease (null; closed symbol). The EC50 (pM) of each treatment was calculated and is depicted in FIG. 25B. As shown in FIG. 25B, each of the masked constructs have an EC50 that is greater than the EC50 of the IL2 control, and there is a decrease in the EC50 of each of the constructs following exposure to the MMP7 protease, as compared to the construct before cleavage. This demonstrates that the exemplary 1032 and 1033 constructs are activatable by an exemplary protease, MMP7.

Binding of exemplary constructs that contain an Fc binding peptide to a humanized Fc-containing IgG1 antibody was assessed using SPR. The exemplary constructs 1031, 1032, and 1033, each of which include either an Fc binding peptide (1031) or an IgG binding protein (1032 and 1033), were tested for their binding to an exemplary humanized Fc-containing IgG1 antibody, trastuzumab. The exemplary construct 329141D, which does not include an Fc binding peptide or an IgG binding protein, was also tested as a negative control. Trastuzumab was immobilized on a CM5 chip (1000 resonance units (RU)) through EDC/NHS coupling. Each of the construct tested (1031, 1032, 1033, and 329141D) were prepared in HBS-EP+ running buffer and the 1031, 1032, and 1033 constructs were used as analyte at concentrations of 300 nM, 100 nM, 30 nM, 10 nM, and 3 nM, and the 329141D construct was used as analyte at concentrations of 300 nM, 100 nM, and 30 nM.

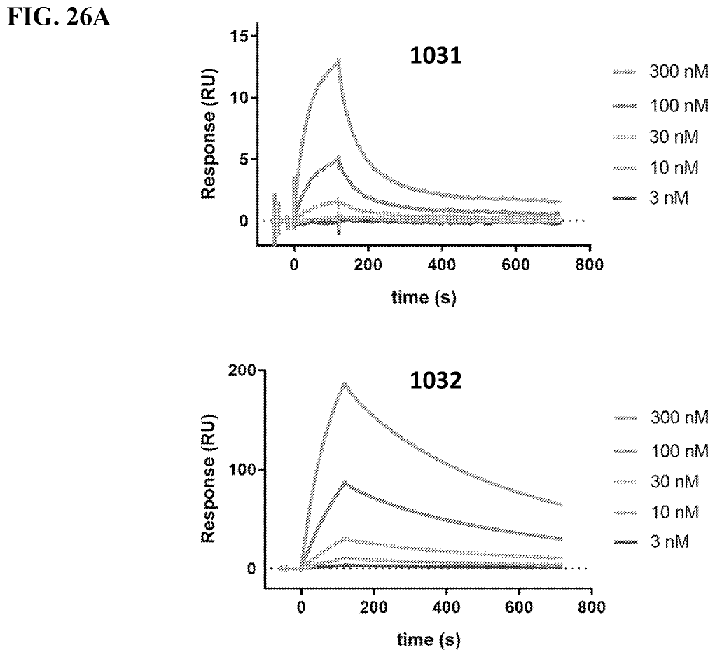

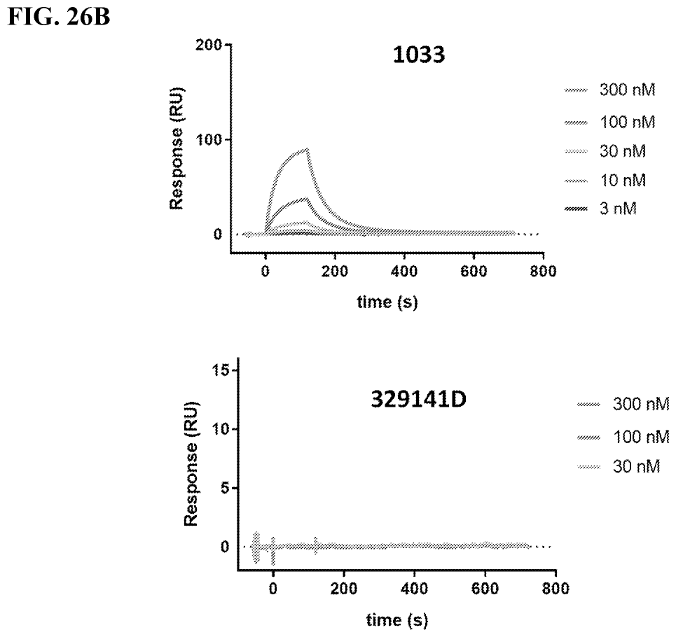

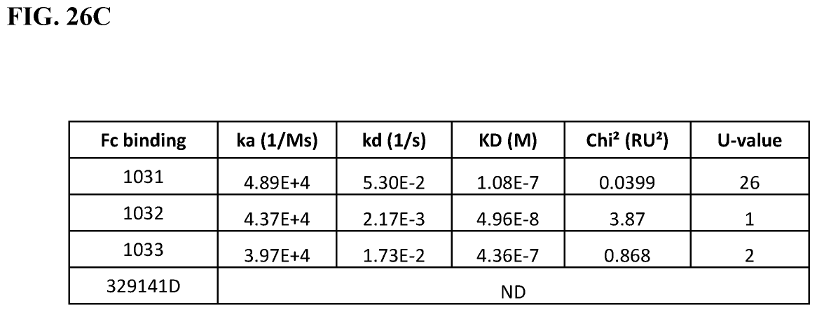

Results from the SPR analyses testing binding of the 1031, 1032, 1033, and 329141D constructs to a humanized IgG antibody (trastuzumab) are depicted in FIGS. 26A-C. FIGS. 26A and 26B depict the interaction between each of the 1031, 1032, 1033, and 329141D constructs and the antibody trastuzumab. FIG. 26C provides data on the association rate (ka), dissociation rate (kd), and equilibrium dissociation constant (KD), as well as Chi² and U-values for the interaction between each construct and trastuzumab, as determined in this analysis. As shown in FIGS. 26A-C, each of the constructs that included either an Fc binding peptide (1031) or an IgG binding protein (1032 and 1033) was shown to bind to the humanized Fc-containing antibody trastuzumab, while the 329141D construct, which did not include an Fc binding peptide or an IgG binding protein, was not shown to bind to the humanized Fc-containing antibody trastuzumab. This demonstrates, among other things, that masked constructs that include an Fc binding peptide and either IL2 or IL15 are capable of binding to an IgG, and that including an IgG binding protein is a strategy for promoting binding of the construct to an IgG.

Binding of exemplary constructs that contain an Fc binding peptide to a heterodimer of IL2Rβ-Fc and IL-2Rγ-Fc is assessed using SPR. Exemplary constructs that include an Fc binding peptide (e.g., 1022, 1023, 1024, 1029, 1030, and 1031) are tested for their binding, or lack thereof, to a heterodimer of IL2Rβ-Fc and IL-2Rγ-Fc. A heterodimer of IL2Rβ-Fc and IL-2Rγ-Fc is immobilized on a CM5 chip (1000 resonance units (RU)) through EDC/NHS coupling. Each of the construct tested (e.g., 1022, 1023, 1024, 1029, 1030, and 1031) are prepared in HBS-EP+ running buffer in the presence or absence of an IgG (e.g., trastuzumab) and each construct is used as analyte at concentrations of 300 nM, 100 nM, 30 nM, 10 nM, and 3 nM. The EC50 of each of the constructs under each condition is determined.

### Example 3: In Vivo Analysis of Exemplary Cytokine Receptor Binding Proteins

Constructs having desired characteristics are selected for animal studies. B16F10 melanoma tumors are established in C57BL/6 mice. One group is treated with a construct of the present disclosure, and another group is treated with IL2. Tumors are harvested, supernatant is collected, and any sterically restrained IL2 are activated with the appropriate protease. IL2 levels are measured using human-specific ELISA assay.

B16F10 tumors in C57BL/6 mice are treated with varying doses of a construct of the present disclosure. Relative to tumor volume in a control group (e.g., untreated, or treated with IL2), constructs slow, halt, or reverse tumor growth.

Constructs of the present disclosure are used in combination with one or more additional agents. CT26 tumors are established in BALB/c mice. Combinations of a construct of the present disclosure and 9D9 (optionally, at a suboptimal dose) or J43 are administered to the mice. Relative to tumor volume in a control group (e.g., untreated, treated with IL2, or treated with one agent of the combination), the combinations slow, halt, or reverse tumor growth.

Kinetic analyses of immune response in healthy mice due to single-injection of a construct are evaluated. Change in immune cell phenotype and cytokine response in vivo are evaluated. Sera and spleens are collected. Sera is tested for TH1 (IFN-γ and TNF-α) and TH2 (IL-5 and IL-10) cytokines using cytokine multiplex analysis. Testing includes a mouse Th1/Th2/Th17 antibody array. Spleens are process into single cell suspensions for antibody staining and flow cytometric analysis of cellular phenotype profile. A variety of monoclonal antibodies are available for characterization of Balb/c splenocytes. Flow cytometric staining of splenocytes in treated mice is used to measure: absolute number of cells in the spleen, number of IL-15 memory responders in the innate and adaptive subsets of CD4⁺ and CD8⁺ T cell populations, and activation status of NK (CD49b⁺) cells.

Cytotoxic function of NK cells harvested from treated healthy mice is also measured.

Immune response and NK cytotoxicity in tumor-bearing mice following single injection of a construct is evaluated. Changes in immune cell phenotype, cytokine response, and NK cell cytotoxicity of treated tumor bearing mice are evaluated. Female Balb/c mic are injected with 5E4 4T1 murine mammary tumor cells. Mice are injected with a construct on day 7. On day 10, sera and spleens are harvested and tested as described above. Anti-tumor activity is also measured in a similar model, or in mice injected with 2E5 CT26 murine colon carcinoma cells. Combinations (e.g., a construct of the present disclosure combined with αCTLA4 and/or αPD-1 monoclonal antibodies) are also made and tested.

1. A recombinant cytokine receptor binding protein comprising:
a cytokine domain comprising a first receptor binding site and a second receptor binding site;
a first receptor domain that specifically binds to said first receptor binding site; and
an occlusion domain positioned to sterically hinder binding of said second receptor binding site to a cognate receptor of said second receptor binding site;
wherein:
(a) the C-terminus of said cytokine domain is bound to the N-terminus of said occlusion domain through a first chemical linker; and the C-terminus of said occlusion domain is bound to the N-terminus of said first receptor domain through a second chemical linker; or
(b) the C-terminus of said occlusion domain is bound to the N-terminus of said cytokine domain through a first chemical linker; and the C-terminus of said first receptor domain is bound to the N-terminus of said occlusion domain through a second chemical linker.

2. The recombinant cytokine receptor binding protein of claim 1, wherein said cytokine domain comprises a third receptor binding site and wherein said occlusion domain is positioned to sterically hinder binding of said third receptor binding site to said cognate receptor.

3. The recombinant cytokine receptor binding protein of claim 1, wherein said cytokine domain is an IL-2 domain.

4. The recombinant cytokine receptor binding protein of claim 3, wherein said IL-2 domain comprises the amino acid sequence of SEQ ID NO: 1.

5. The recombinant cytokine receptor binding protein of claim 1, wherein said first receptor domain comprises an amino acid sequence selected from the group consisting of SEQ ID NOs: 5, 50, 59, 90, 100, 101, and 105.

6. The recombinant cytokine receptor binding protein of claim 1, wherein said cytokine domain is an IL-15 domain.

7. The recombinant cytokine receptor binding protein of claim 6, wherein said IL-15 domain comprises the amino acid sequence of SEQ ID NO: 92.

8. The recombinant cytokine receptor binding protein of claim 6, wherein said first receptor domain comprises the amino acid sequence of SEQ ID NO: 94.

9. The recombinant cytokine receptor binding protein of claim 1, wherein said occlusion domain is an Fc domain, a Fab domain, a nanobody domain, a protein L domain, or a CD16 domain.

10. The recombinant cytokine receptor binding protein of claim 1, wherein said occlusion domain comprises an amino acid sequence selected from the group consisting of SEQ ID NOs: 3, 51, 52, 88, 89, 91, 93, 95-98, 102, 103, and 107.

11. The recombinant cytokine receptor binding protein of claim 1, wherein said first chemical linker comprises an amino acid sequence selected from the group consisting of SEQ ID NOs: 2, 4, 9-25, 29, 34-37, 61-64, and 104, and/or said second chemical linker comprises an amino acid sequence selected from the group consisting of SEQ ID NOs: 2, 4, 9-25, 29, 34-37, 61-64, and 104.

12. The recombinant cytokine receptor binding protein of claim 1, wherein said first chemical linker or said second chemical linker is a cleavable linker.

13. The recombinant cytokine receptor binding protein of claim 12, wherein said cleavable linker comprises an amino acid sequence selected from the group consisting of SEQ ID NOs: 2, 23-25, 34, 36, 37, 61, and 62.

14. The recombinant cytokine receptor binding protein of claim 1, wherein said recombinant cytokine receptor binding protein comprises an Fc binding peptide.

15. The recombinant cytokine receptor binding protein of claim 14, wherein said Fc binding peptide comprises an amino acid sequence selected from the group consisting of SEQ ID NOs: 31-33.

16. The recombinant cytokine receptor binding protein of claim 1, wherein said recombinant cytokine receptor binding protein comprises an amino acid sequence selected from the group consisting of SEQ ID NOs: 6, 26-28, 30, 38-48, 53-56, 60, 65-87, 99, 106, and 108-119.

17. A recombinant cytokine receptor binding protein comprising:
an IL-2 domain comprising an IL-2 receptor α binding site, an IL-2 receptor β binding site and an IL-2 receptor γ binding site;
an IL-2 receptor α domain that specifically binds to said IL-2 receptor α binding site; and
an Fc domain positioned to sterically hinder binding of said IL-2 receptor β binding site to an IL-2 receptor β domain; wherein the C-terminus of said IL-2 domain is bound to the N-terminus of said Fc domain through a cleavable linker; and
the C-terminus of said Fc domain is bound to the N-terminus of said IL2 receptor α domain through a chemical linker.

18. The recombinant cytokine receptor binding protein of claim 17, wherein said Fc domain is positioned to sterically hinder binding of said IL-2 receptor γ binding site to an IL-2 receptor γ domain.

19. The recombinant cytokine receptor binding protein of claim 17, wherein said IL-2 domain comprises the amino acid sequence of SEQ ID NO: 1.

20. The recombinant cytokine receptor binding protein of claim 17, wherein said IL-2 receptor α domain comprises an amino acid sequence selected from the group consisting of SEQ ID NOs: 5, 50, 59, 90, 100, 101, and 105.

21. The recombinant cytokine receptor binding protein of claim 17, wherein said Fc domain comprises an amino acid sequence selected from the group consisting of SEQ ID NOs: 3, 88, 89, 91, 93, 103, and 107.

22. The recombinant cytokine receptor binding protein of claim 17, wherein said cleavable linker comprises an amino acid sequence selected from the group consisting of SEQ ID NOs: 2, 23-25, 29, 34, 36, 37, 61, 62, and 64.

23. The recombinant cytokine receptor binding protein of claim 17, wherein said chemical linker comprises an amino acid sequence selected from the group consisting of SEQ ID NOs: 4, 9, 11, 12, 14, 15, 17, 18, 20, 21, 24, 35, and 63.

24. The recombinant cytokine receptor binding protein of claim 17, wherein said recombinant cytokine receptor binding protein comprises an Fc binding peptide.

25. The recombinant cytokine receptor binding protein of claim 24, wherein said Fc binding peptide comprises an amino acid sequence selected from the group consisting of SEQ ID NOs: 31-33.

26. A pharmaceutical composition comprising a recombinant cytokine receptor binding protein of claim 1 and a pharmaceutically acceptable excipient.

27. An isolated nucleic acid encoding a recombinant cytokine receptor binding protein of claim 1.

28. An expression vector comprising the nucleic acid of claim 27.

29. A T lymphocyte comprising the expression vector of claim 28.

30. A method of treating cancer in subject in need thereof, said method comprising administering to a subject a therapeutically effective amount of a recombinant cytokine receptor binding protein of claim 1.


SEQUENCE LISTING (119 sequences)

SEQ ID NO: 1 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2 - protein, 10 aa, Artificial Sequence
  Synthetic Construct
GGVPLSLYSG

SEQ ID NO: 3 - protein, 109 aa, Homo sapiens
NGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQP

SEQ ID NO: 4 - protein, 7 aa, Artificial Sequence
  Synthetic Construct
GASASGG

SEQ ID NO: 5 - protein, 171 aa, Homo sapiens
GMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 6 - protein, 442 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPGASASGGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 7 - DNA, 4653 bp, Artificial Sequence
  Synthetic Construct
attacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttggatatggctcattttttacttcctcaccttgtcgtattatactatgccgatatactatgccgatgattaattgtcgacactgcgggggctctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccgcgtctcaggggcgagacctctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagagggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaagccgccaccatgggctggtccctgatcctgctgttcctggtggccgtggccacgcgtgtgctgtcagcacctaccagctcctccaccaaaaagacccagctgcagttggagcatctcctcttggatctgcaaatgattctgaacggaatcaacaattacaagaacccaaagctgactagaatgctgaccttcaagttctacatgccgaagaaagccaccgagctgaagcaccttcagtgcctcgaggaagaactcaagcccctggaagaagtgctgaacctggcgcagtccaagaacttccaccttcggccgcgggacctcatctccaacatcaacgtcatcgtgctggaactcaagggttccgagactacctttatgtgcgaatacgccgacgagactgcgaccattgtcgagttcctgaaccgctggatcacattcgctcagtccattatcagcaccttgactggcggcgtgcctctgtccctgtactctggaaacgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtcagcaacaaagccctcccagcccccatcgagaaaaccatctccaaagccaaagggcagcccggagcctccgcatcgggaggaggaatgctctcccttgagctctgcgacgatgatccgcccgaaatcccgcatgccacattcaaggccatggcgtacaaggaaggcaccatgctgaactgtgaatgcaaacggggcttccggaggattaagtctggatccttgtacatgctctgcaccgggtcgtcgagccactccagctgggacaaccagtgccagtgtacctcctccgccacccgctccaccaccaaacaagtcactccccaacctgaggaacagaaggagagaaagactaccgagatgcagtcgccaatgcagccagtggaccaggcctcactccccggacactgtcgggagcctccgccgtgggagaacgaagctactgaacgcatctaccactttgtcgtgggtcagatggtgtactaccaatgcgtgcagggatatagagcccttcaccgcggaccggcagagtccgtgtgcaagatgacccatggaaagacccgctggacccagccccaactgatttgcactggcgaagccagcggcggtggaggccaccaccaccaccatcactaaggttgaggtctcaaaaactgtcttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagacagtcagccttgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggaca

SEQ ID NO: 8 - DNA, 1446 bp, Artificial Sequence
  Synthetic Construct
attacaaacaggaatcgagtgcaaccggcgcaggaacactgccagcgcatcaacaatattatgggctggtccctgatcctgctgttcctggtggccgtggccacgcgtgtgctgtcagcacctaccagctcctccaccaaaaagacccagctgcagttggagcatctcctcttggatctgcaaatgattctgaacggaatcaacaattacaagaacccaaagctgactagaatgctgaccttcaagttctacatgccgaagaaagccaccgagctgaagcaccttcagtgcctcgaggaagaactcaagcccctggaagaagtgctgaacctggcgcagtccaagaacttccaccttcggccgcgggacctcatctccaacatcaacgtcatcgtgctggaactcaagggttccgagactacctttatgtgcgaatacgccgacgagactgcgaccattgtcgagttcctgaaccgctggatcacattcgctcagtccattatcagcaccttgactggcggcgtgcctctgtccctgtactctggaaacgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtcagcaacaaagccctcccagcccccatcgagaaaaccatctccaaagccaaagggcagcccggagcctccgcatcgggaggaggaatgctctcccttgagctctgcgacgatgatccgcccgaaatcccgcatgccacattcaaggccatggcgtacaaggaaggcaccatgctgaactgtgaatgcaaacggggcttccggaggattaagtctggatccttgtacatgctctgcaccgggtcgtcgagccactccagctgggacaaccagtgccagtgtacctcctccgccacccgctccaccaccaaacaagtcactccccaacctgaggaacagaaggagagaaagactaccgagatgcagtcgccaatgcagccagtggaccaggcctcactccccggacactgtcgggagcctccgccgtgggagaacgaagctactgaacgcatctaccactttgtcgtgggtcagatggtgtactaccaatgcgtgcagggatatagagcccttcaccgcggaccggcagagtccgtgtgcaagatgacccatggaaagacccgctggacccagccccaactgatttgcactggcgaagccagcggcggtggaggccaccaccaccaccatcactaa

SEQ ID NO: 9 - protein, 16 aa, Artificial Sequence
  Synthetic Construct
KGQPGASASGGGMLSL

SEQ ID NO: 10 - protein, 8 aa, Artificial Sequence
  Synthetic Construct
GASASGGG

SEQ ID NO: 11 - protein, 9 aa, Artificial Sequence
  Synthetic Construct
GGASASAGG

SEQ ID NO: 12 - protein, 14 aa, Artificial Sequence
  Synthetic Construct
KGQPGASASGMLSL

SEQ ID NO: 13 - protein, 6 aa, Artificial Sequence
  Synthetic Construct
GASASG

SEQ ID NO: 14 - protein, 12 aa, Artificial Sequence
  Synthetic Construct
KGGASASGMLSL

SEQ ID NO: 15 - protein, 12 aa, Artificial Sequence
  Synthetic Construct
SGGASASGSLSL

SEQ ID NO: 16 - protein, 10 aa, Artificial Sequence
  Synthetic Construct
SGGASASGSL

SEQ ID NO: 17 - protein, 14 aa, Artificial Sequence
  Synthetic Construct
SGGASASGSLSLEL

SEQ ID NO: 18 - protein, 10 aa, Artificial Sequence
  Synthetic Construct
SGGASADGDG

SEQ ID NO: 19 - protein, 6 aa, Artificial Sequence
  Synthetic Construct
GGGSGG

SEQ ID NO: 20 - protein, 11 aa, Artificial Sequence
  Synthetic Construct
GGASGSGAGAG

SEQ ID NO: 21 - protein, 9 aa, Artificial Sequence
  Synthetic Construct
GGGSGAGAG

SEQ ID NO: 22 - protein, 10 aa, Artificial Sequence
  Synthetic Construct
GGSPSGASGS

SEQ ID NO: 23 - protein, 11 aa, Artificial Sequence
  Synthetic Construct
GGVPLSLYSGN

SEQ ID NO: 24 - protein, 13 aa, Artificial Sequence
  Synthetic Construct
GGGGVPLSLYSGG

SEQ ID NO: 25 - protein, 11 aa, Artificial Sequence
  Synthetic Construct
GGVPLSLYSGG

SEQ ID NO: 26 - protein, 319 aa, Artificial Sequence
  Synthetic Construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLITCPPPMSVEHADIWVKSYSLYSRERYICNSGFKRKAGTSSLTECVLNKATNVAHWTTPSLKCIRDPALVHQRPAPPS

SEQ ID NO: 27 - protein, 394 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 28 - protein, 442 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGDCAWHLGELVWCTGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 29 - protein, 6 aa, Artificial Sequence
  Synthetic Construct
VPLSLY

SEQ ID NO: 30 - protein, 443 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAGGDCAWHLGELVWCTGGSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 31 - protein, 13 aa, Artificial Sequence
  Synthetic Construct
DCAWHLGELVWCT

SEQ ID NO: 32 - protein, 15 aa, Artificial Sequence
  Synthetic Construct
GGDCAWHLGELVWCT

SEQ ID NO: 33 - protein, 15 aa, Artificial Sequence
  Synthetic Construct
DCAWHLGELVWCTGG

SEQ ID NO: 34 - protein, 28 aa, Artificial Sequence
  Synthetic Construct
GGVPLSLYSGNGGDCAWHLGELVWCTGG

SEQ ID NO: 35 - protein, 29 aa, Artificial Sequence
  Synthetic Construct
GGDCAWHLGELVWCTGGSGGASASGSLSL

SEQ ID NO: 36 - protein, 30 aa, Artificial Sequence
  Synthetic Construct
GGVPLSLYSGNGGGGDCAWHLGELVWCTGG

SEQ ID NO: 37 - protein, 30 aa, Artificial Sequence
  Synthetic Construct
GGVPLSLYSGNGGDCAWHLGELVWCTGGGG

SEQ ID NO: 38 - protein, 442 aa, Artificial Sequence; identical to SEQ ID NO: 6
  Synthetic Construct

SEQ ID NO: 39 - protein, 435 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAGGASASAGGELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 40 - protein, 440 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPGASASGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 41 - protein, 438 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGGASASGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 42 - protein, 438 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 43 - protein, 452 aa, Artificial Sequence
  Synthetic Construct
GGDCAWHLGELVWCTGGSAASGASGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 44 - protein, 529 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 45 - protein, 529 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 46 - protein, 423 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGSPSGASGSPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 47 - protein, 424 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASADGDGELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 48 - protein, 444 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGGGDCAWHLGELVWCTGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 49 - protein, 1329 aa, Artificial Sequence
  Synthetic Construct
GCACCTACCAGCTCCTCCACCAAAAAGACCCAGCTGCAGTTGGAGCATCTCCTCTTGGATCTGCAAATGATTCTGAACGGAATCAACAATTACAAGAACCCAAAGCTGACTAGAATGCTGACCTTCAAGTTCTACATGCCGAAGAAAGCCACCGAGCTGAAGCACCTTCAGTGCCTCGAGGAAGAACTCAAGCCCCTGGAAGAAGTGCTGAACCTGGCGCAGTCCAAGAACTTCCACCTTCGGCCGCGGGACCTCATCTCCAACATCAACGTCATCGTGCTGGAACTCAAGGGTTCCGAGACTACCTTTATGTGCGAATACGCCGACGAGACTGCGACCATTGTCGAGTTCCTGAACCGCTGGATCACATTCGCTCAGTCCATTATCAGCACCTTGACTGGCGGCGTGCCTCTGTCCCTGTACTCTGGAAACGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGGGTGGTCAGCGTCCTCACCGTCCTGCACCAGGACTGGCTGAATGGCAAGGAGTACAAGTGCAAGGTCAGCAACAAAGCCCTCCCAGCCCCCATCGAGAAAACCATCTCCAAAGCCAAAGGGCAGCCCGGAGCCTCCGCATCGGGAGGAGGAATGCTCTCCCTTGAGCTCTGCGACGATGATCCGCCCGAAATCCCGCATGCCACATTCAAGGCCATGGCGTACAAGGAAGGCACCATGCTGAACTGTGAATGCAAACGGGGCTTCCGGAGGATTAAGTCTGGATCCTTGTACATGCTCTGCACCGGGTCGTCGAGCCACTCCAGCTGGGACAACCAGTGCCAGTGTACCTCCTCCGCCACCCGCTCCACCACCAAACAAGTCACTCCCCAACCTGAGGAACAGAAGGAGAGAAAGACTACCGAGATGCAGTCGCCAATGCAGCCAGTGGACCAGGCCTCACTCCCCGGACACTGTCGGGAGCCTCCGCCGTGGGAGAACGAAGCTACTGAACGCATCTACCACTTTGTCGTGGGTCAGATGGTGTACTACCAATGCGTGCAGGGATATAGAGCCCTTCACCGCGGACCGGCAGAGTCCGTGTGCAAGATGACCCATGGAAAGACCCGCTGGACCCAGCCCCAACTGATTTGCACTGGCGAAGCCAGCGGCGGTGGAGGCCACCACCACCACCATCACTAA

SEQ ID NO: 50 - protein, 173 aa, Homo sapiens
GMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEAS

SEQ ID NO: 51 - protein, 214 aa, Artificial Sequence
  Synthetic Construct
DIQMTQSPILLSASVGDRVTITCRASQDVNTAVAWYQQRTNGSPRLLIYSASFLYSGVPSRFSGSRSGTDFTLTISSLQPEDIADYYCQQHYTTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 223 aa, Artificial Sequence
  Synthetic Construct
EVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 53 - protein, 539 aa, Artificial Sequence
  Synthetic Construct
GMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGSGGDIQMTQSPILLSASVGDRVTITCRASQDVNTAVAWYQQRTNGSPRLLIYSASFLYSGVPSRFSGSRSGTDFTLTISSLQPEDIADYYCQQHYTTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGVPLSLYSGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 54 - protein, 548 aa, Artificial Sequence
  Synthetic Construct
GMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGSGGEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGVPLSLYSGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 55 - protein, 542 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGDIQMTQSPILLSASVGDRVTITCRASQDVNTAVAWYQQRTNGSPRLLIYSASFLYSGVPSRFSGSRSGTDFTLTISSLQPEDIADYYCQQHYTTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGASGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEAS

SEQ ID NO: 56 - protein, 549 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEAS

SEQ ID NO: 57 - protein, 12 aa, Artificial Sequence
  Synthetic Construct
ASGGGGHHHHHH

SEQ ID NO: 58 - protein, 6 aa, Artificial Sequence
  Synthetic Construct
HHHHHH

SEQ ID NO: 59 - protein, 183 aa, Artificial Sequence
  Synthetic Construct
GMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 60 - protein, 461 aa, Artificial Sequence
  Synthetic Construct
MGWSLILLFLVAVATRVLSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPGASASGGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 61 - protein, 9 aa, Artificial Sequence
  Synthetic Construct
GVPLSLYSG

SEQ ID NO: 62 - protein, 14 aa, Artificial Sequence
  Synthetic Construct
GGSPLAQAVRSSSG

SEQ ID NO: 63 - protein, 10 aa, Artificial Sequence
  Synthetic Construct
GGGSGAGAGG

SEQ ID NO: 64 - protein, 10 aa, Artificial Sequence
  Synthetic Construct
SPLAQAVRSS

SEQ ID NO: 65 - protein, 552 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGDIQMTQSPILLSASVGDRVTITCRASQDVNTAVAWYQQRTNGSPRLLIYSASFLYSGVPSRFSGSRSGTDFTLTISSLQPEDIADYYCQQHYTTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGASGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 66 - protein, 559 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 67 - protein, 539 aa, Artificial Sequence; identical to SEQ ID NO: 53
  Synthetic Construct

SEQ ID NO: 68 - protein, 548 aa, Artificial Sequence; identical to SEQ ID NO: 54
  Synthetic Construct

SEQ ID NO: 69 - protein, 562 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGSPLAQAVRSSSGEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 70 - protein, 564 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGEVQLVESGGGLVQPGGSLRLSCAASGFTLSGDWIHWVRQSPGKGLEWLGEISAAGGYTDYADSVKGRFTISADTSKNTAYLQMNSLRAEDTAIYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 71 - protein, 331 aa, Artificial Sequence
  Synthetic Construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLITCPPPMSVEHADIWVKSYSLYSRERYICNSGFKRKAGTSSLTECVLNKATNVAHWTTPSLKCIRDPALVHQRPAPPSASGGGGHHHHHH

SEQ ID NO: 72 - protein, 406 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 73 - protein, 454 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGDCAWHLGELVWCTGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 74 - protein, 455 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAGGDCAWHLGELVWCTGGSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 75 - protein, 464 aa, Artificial Sequence
  Synthetic Construct
GGDCAWHLGELVWCTGGSAASGASGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 76 - protein, 541 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 77 - protein, 541 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 78 - protein, 435 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGSPSGASGSPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 79 - protein, 436 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASADGDGELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 80 - protein, 456 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGGGDCAWHLGELVWCTGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 81 - protein, 456 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGDCAWHLGELVWCTGGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 82 - protein, 349 aa, Artificial Sequence
  Synthetic Construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGVPLSLYSGNGGDCAWHLGELVWCTGGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLITCPPPMSVEHADIWVKSYSLYSRERYICNSGFKRKAGTSSLTECVLNKATNVAHWTTPSLKCIRDPALVHQRPAPPSASGGGGHHHHHH

SEQ ID NO: 83 - protein, 455 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNQVQLQESGGGLVQPGGSLRLSCAASGRTISRYAMSWFRQAPGKEREFVAVARRSGDGAFYADSVQGRFTVSRDDAKNTVYLQMNSLKPEDTAVYYCAIDSDTFYSGSYDYWGQGTQVTVSSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 84 - protein, 399 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNSGSEVTIKVNLIFADGKIQTAEFKGTFEEATAEAYRYAALLAKVNGEYTADLEDGGNHMNIKFAGSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 85 - protein, 463 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNQVQLQESGGGLVQAGGSLRLSCAVSGNTLSRYAMGWFRQTPGNEREFVAAIRWNNGATYYADSVEGRFTISRDSGKNTVYLQMNNLQPEDTAVYYCAGRFLGYASSNAYHEALYNYDYWGQGTQVTVSSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 86 - protein, 525 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNMRTEDLPKAEVILEPQWNRVLEKDSVTLKCRGAYSPEDQSTQWFHNESLISSQASSYLIDAATVDDSGEYRCQTSLSTLSDPVQLEVHIGWLLLQAPRWEFKEGDPIHLRCHSWKNTALHKVTYLQNGKGRKYFHHNSDFYIPKATLKDSGSYSCRGLVGSKNVSSETVQITITQGLAVSTISSFFPPGYQSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 87 - protein, 550 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGSPLAQAVRSSSGEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 88 - protein, 73 aa, Homo sapiens
NGEDPEVKFNWYVDGVEVHNAKTKPREEQYNSTYRVVSVLTVLHQDWLNGKEYKCKVSNKALPAPIEKTISKA

SEQ ID NO: 89 - protein, 208 aa, Homo sapiens
NGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLS

SEQ ID NO: 90 - protein, 166 aa, Homo sapiens
ELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 91 - protein, 208 aa, Artificial Sequence
  Synthetic Construct
NGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLS

SEQ ID NO: 92 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 93 - protein, 103 aa, Homo sapiens
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKA

SEQ ID NO: 94 - protein, 78 aa, Homo sapiens
ITCPPPMSVEHADIWVKSYSLYSRERYICNSGFKRKAGTSSLTECVLNKATNVAHWTTPSLKCIRDPALVHQRPAPPS

SEQ ID NO: 95 - protein, 122 aa, Artificial Sequence
  Synthetic Construct
NQVQLQESGGGLVQPGGSLRLSCAASGRTISRYAMSWFRQAPGKEREFVAVARRSGDGAFYADSVQGRFTVSRDDAKNTVYLQMNSLKPEDTAVYYCAIDSDTFYSGSYDYWGQGTQVTVSS

SEQ ID NO: 96 - protein, 66 aa, Artificial Sequence
  Synthetic Construct
NSGSEVTIKVNLIFADGKIQTAEFKGTFEEATAEAYRYAALLAKVNGEYTADLEDGGNHMNIKFAG

SEQ ID NO: 97 - protein, 130 aa, Artificial Sequence
  Synthetic Construct
NQVQLQESGGGLVQAGGSLRLSCAVSGNTLSRYAMGWFRQTPGNEREFVAAIRWNNGATYYADSVEGRFTISRDSGKNTVYLQMNNLQPEDTAVYYCAGRFLGYASSNAYHEALYNYDYWGQGTQVTVSS

SEQ ID NO: 98 - protein, 192 aa, Homo sapiens
NMRTEDLPKAEVILEPQWNRVLEKDSVTLKCRGAYSPEDQSTQWFHNESLISSQASSYLIDAATVDDSGEYRCQTSLSTLSDPVQLEVHIGWLLLQAPRWEFKEGDPIHLRCHSWKNTALHKVTYLQNGKGRKYFHHNSDFYIPKATLKDSGSYSCRGLVGSKNVSSETVQITITQGLAVSTISSFFPPGYQ

SEQ ID NO: 99 - protein, 444 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGDCAWHLGELVWCTGGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 100 - protein, 173 aa, Homo sapiens; identical to SEQ ID NO: 50

SEQ ID NO: 101 - protein, 170 aa, Homo sapiens
MLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 102 - protein, 228 aa, Artificial Sequence
  Synthetic Construct
EVQLVESGGGLVQPGGSLRLSCAASGFTLSGDWIHWVRQSPGKGLEWLGEISAAGGYTDYADSVKGRFTISADTSKNTAYLQMNSLRAEDTAIYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVDKKVEPKSC

SEQ ID NO: 103 - protein, 105 aa, Homo sapiens
NGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKA

SEQ ID NO: 104 - protein, 11 aa, Artificial Sequence
  Synthetic Construct
GGSAASGASGG

SEQ ID NO: 105 - protein, 183 aa, Artificial Sequence; identical to SEQ ID NO: 59
  Synthetic Construct

SEQ ID NO: 106 - protein, 461 aa, Artificial Sequence; identical to SEQ ID NO: 60
  Synthetic Construct

SEQ ID NO: 107 - protein, 102 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKA

SEQ ID NO: 108 - protein, 430 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPGASASGGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 109 - protein, 423 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAGGASASAGGELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 110 - protein, 428 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPGASASGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 111 - protein, 426 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGGASASGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 112 - protein, 426 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 113 - protein, 337 aa, Artificial Sequence
  Synthetic Construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGVPLSLYSGNGGDCAWHLGELVWCTGGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKASGGASASGSLSLITCPPPMSVEHADIWVKSYSLYSRERYICNSGFKRKAGTSSLTECVLNKATNVAHWTTPSLKCIRDPALVHQRPAPPS

SEQ ID NO: 114 - protein, 443 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNQVQLQESGGGLVQPGGSLRLSCAASGRTISRYAMSWFRQAPGKEREFVAVARRSGDGAFYADSVQGRFTVSRDDAKNTVYLQMNSLKPEDTAVYYCAIDSDTFYSGSYDYWGQGTQVTVSSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 115 - protein, 387 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNSGSEVTIKVNLIFADGKIQTAEFKGTFEEATAEAYRYAALLAKVNGEYTADLEDGGNHMNIKFAGSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 116 - protein, 451 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNQVQLQESGGGLVQAGGSLRLSCAVSGNTLSRYAMGWFRQTPGNEREFVAAIRWNNGATYYADSVEGRFTISRDSGKNTVYLQMNNLQPEDTAVYYCAGRFLGYASSNAYHEALYNYDYWGQGTQVTVSSSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 117 - protein, 513 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGNMRTEDLPKAEVILEPQWNRVLEKDSVTLKCRGAYSPEDQSTQWFHNESLISSQASSYLIDAATVDDSGEYRCQTSLSTLSDPVQLEVHIGWLLLQAPRWEFKEGDPIHLRCHSWKNTALHKVTYLQNGKGRKYFHHNSDFYIPKATLKDSGSYSCRGLVGSKNVSSETVQITITQGLAVSTISSFFPPGYQSGGASASGSLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 118 - protein, 552 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGEVQLVESGGGLVQPGGSLRLSCAASGFTLSGDWIHWVRQSPGKGLEWLGEISAAGGYTDYADSVKGRFTISADTSKNTAYLQMNSLRAEDTAIYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 119 - protein, 547 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE